(19) United States
(12) Patent Application Publication
(10) Pub. No.: US 2015/0045284 A1
(43) Pub. Date: Feb. 12, 2015

(54) CRYSTAL STRUCTURE OF A TYPE IB P-TYPE ATPase

(71) Applicants: Poul Nissen, Risskov (DK); Pontus Gourdon, Arhus C (DK); Xiang Liu, Peking (CN); Bjorn Panyella Pedersen, San Francisco, CA (US); Daniel Mattle, Risskov (DK)

(72) Inventors: Poul Nissen, Risskov (DK); Pontus Gourdon, Arhus C (DK); Xiang Liu, Peking (CN); Bjorn Panyella Pedersen, San Francisco, CA (US); Daniel Mattle, Risskov (DK)

(73) Assignee: Aarhus Universitet, Arhus C (DK)

(21) Appl. No.: 13/980,626

(22) PCT Filed: Jan. 20, 2012
(86) PCT No.: PCT/DK2012/050026

§ 371 Date: Jul. 19, 2013

(30) Foreign Application Priority Data
Jan. 21, 2011 (DK) PA 2011 70035

(51) Int. Cl. C12N 9/99; G01N 33/573; G06F 19/16; C12N 9/14

(52) U.S. Cl. CPC C12N 9/99; C12N 9/14; G01N 33/573; G06F 19/16; C12Y 306/00; G01N 2500/04
USPC 514/2.4; 435/195; 435/18; 435/7.4; 506/9; 530/300; 703/11

(57) ABSTRACT

The present invention relates to crystals of a type IB P-type ATPase having the space group P1 and methods for purification and growing said crystals. The invention also presents methods for identifying an inhibitor of a type IB P-type ATPase for example by determining binding interactions between an inhibitor and a set of binding interaction sites in said type IB P-type ATPase.

### FIELD OF INVENTION

The present invention relates to crystals of a type IB P-type ATPase having the space group P1 and methods for purification and growing said crystals. The invention also presents methods for identifying an inhibitor of a type IB P-type ATPase for example by determining binding interactions between an inhibitor and a set of binding interaction sites in said type IB P-type ATPase.

### BACKGROUND OF INVENTION

Heavy metal homeostasis and detoxification is crucial for cell viability and type IB P-type ATPases play an essential role in these processes through the active extrusion of heavy metals from the cytoplasm of cells. P-type ATPases are integral membrane pumps that derive energy from ATP hydrolysis to maintain ion homeostasis and lipid bilayer asymmetry in cells. They form a superfamily that encompasses eleven distinct classes, of which class IB and IIA are the largest and most widespread. Atomic structures have only been determined from three classes: The class IIA Sarcoplasmic reticulum Ca²⁺-ATPase (SERCA1a from rabbit skeletal muscle) in multiple conformations (Toyoshima, C. and Nomura, H., Nature 418 (6898), 605 (2002); Olesen, C. et al., Science 306 (5705), 2251 (2004); Toyoshima, C., Nomura, H., and Tsuda, T., Nature 432 (7015), 361 (2004)), the class IIC Na⁺,K⁺-ATPase (from pig kidney and spiny dogfish) (Morth, J. P. et al., Nature 450 (7172), 1043 (2007); Shinoda, T., Ogawa, H., Cornelius, F., and Toyoshima, C., Nature 459 (7245), 446 (2009)), and the class IIIA H⁺-ATPase (from Arabidopsis thaliana) (Pedersen, B. P. et al., Nature 450 (7172), 1111 (2007)). The catalytic mechanism of the P-type ATPases is described by the E1/E2 Albers-Post model (Albers, R. W., Annu Rev Biochem 36, 727 (1967); Post, R. L., Hegyvary, C., and Kume, S., J Biol Chem 247 (20), 6530 (1972)) with E1 and E2 states associated with high and low affinity for the extruded substrate, and E1P and E2P denoting phosphoenzyme intermediates.

Class IB comprises the heavy metal transporting ATPases, which are essential cellular regulators of Cu⁺, Zn²⁺ and Co²⁺ ions among others. Tight regulation and active transport is an essential process due to the toxicity of these metals of which some are indispensable as cofactors in a variety of enzymes. Reflecting the low intracellular concentrations of these ions, the heavy metal ATPases are characterized by high apparent affinity in the femtomolar-range as well as a slow reaction cycle compared to cation pumps such as SERCA1a (Mandal, A. K., Cheung, W. D., and Arguello, J. M., J Biol Chem 277 (9), 7201 (2002)). Class IB displays a conserved intramembranous CPC motif associated with metal binding. Structurally, these pumps have been predicted to share the core of the fold of P-type ATPases, with a transmembrane domain (M) and three cytoplasmic domains A, P, N, albeit only the first six out of ten transmembrane helices from other classes are present in class IB. Two additional transmembrane helices are typically predicted to be located before the A-domain, although the exact topology has remained uncertain. Furthermore, the heavy metal ATPases possess one or more sequential heavy metal-binding domains (HMBD) in one or both of the termini (Arguello, J. M., Eren, E., and Gonzalez-Guerrero, M., Biometals 20 (3-4), 233 (2007)). It is still not established how and when these domains interact with the catalytic core, if at all, and if they participate in subcellular targeting (eukaryotes only), regulation, and/or ion transfer.

Cu⁺-transporting ATPases are the most prevalent heavy metal ATPases. In plants and many microorganisms they play an important role in detoxification; in A. thaliana four out of eight P1B-type ATPases are Cu⁺-ATPases. The two class IB ATPases in human, ATP7A and ATP7B (also known as Menkes and Wilson proteins), are Cu⁺-ATPases as well.

Atomic structures of the cytoplasmic domains from heavy metal ATPases exist, revealing IB-specific features (Sazinsky, M. H., Agarwal, S., Arguello, J. M., and Rosenzweig, A. C., Biochemistry 45 (33), 9949 (2006); Sazinsky, M. H., Mandal, A. K., Arguello, J. M., and Rosenzweig, A. C., J Biol Chem 281 (16), 11161 (2006); Tsuda, T. and Toyoshima, C., EMBO J 28 (12), 1782 (2009)). Electron microscopy structures have also been determined, however reaching somewhat contrasting models (Wu, C. C., Rice, W. J., and Stokes, D. L., Structure 16 (6), 976 (2008); Chintalapati, S., Al Kurdi, R., van Scheltinga, A. C., and Kuhlbrandt, W., J Mol Biol 378 (3), 581 (2008)), but no atomic structure of the complete enzyme exist until now. Thus, key questions on the membrane domain topology and how functional coupling is established to copper transport remain to be answered. The present invention reports the first atomic structures of a complete class IB P-type ATPase and provides critical new insight to address these questions. The structures of the present invention further provides a model for designing inhibitors of class IB P-type ATPases.

### SUMMARY OF INVENTION

One aspect of the present invention relates to a crystal comprising a type IB P-type ATPase, wherein said crystal is characterised in having the space group P1.

The type IB P-type ATPase may form a complex with an organic compound selected from the group consisting of: ATP, ATP analogues, ADP and ADP analogues. Also, the type IB P-type ATPase may form a complex with AlF₄⁻ or other compounds containing fluoride.

Thus, in one preferred embodiment the type IB P-type ATPase forms a complex with AlF₄⁻. In another preferred embodiment the type IB P-type ATPase forms a complex with MgF₄²⁻. In yet another preferred embodiment the type IB P-type ATPase forms a complex with BeF₃⁻.

In a first embodiment the crystal is characterised in having the space group P1 with the unit cell parameters a=44 Å±3 Å, b=80 Å±4 Å, c=330 Å±20 Å and/or in having the space group P2₁2₁2₁. P2₁2₁2₁ is also referred to as the pseudo-symmetry of the crystal. In a preferred embodiment, the crystal having the unit cell parameters a=44 Å±3 Å, b=80 Å±4 Å, c=330 Å±20 Å forms a complex with AlF₄⁻. In a particular embodiment, the crystal has the unit cell parameters a=44 Å±3 Å, b=80 Å±4 Å, c=330 Å±20 Å, the space group P2₁2₁2₁ and forms a complex with AlF₄⁻.

In a second embodiment the crystal is characterised in having the space group P1 with the unit cell parameters a=44 Å±3 Å, b=73 Å±4 Å, c=329 Å±20 Å and/or in having the space group P2₁2₁2₁. P2₁2₁2₁ is also referred to as the pseudo-symmetry of the crystal. In a preferred embodiment, the crystal having the unit cell parameters a=44 Å±3 Å, b=73 Å±4 Å, c=329 Å±20 Å forms a complex with MgF₄²⁻. In a particular embodiment, the crystal has the unit cell parameters a=44 Å±3 Å, b=73 Å±4 Å, c=329 Å±20 Å, the space group P2₁2₁2₁ and forms a complex with MgF₄²⁻.

In a third embodiment the crystal is characterised in having the space group P1 with the unit cell parameters a=242 Å±15 Å, b=71 Å±4 Å, c=72 Å±4 Å and/or in having the space group C₂. C₂ is also referred to as the pseudo-symmetry of the crystal. In a preferred embodiment, the crystal having the unit cell parameters a=242 Å±15 Å, b=71 Å±4 Å, c=72 Å±4 Å forms a complex with BeF₃⁻. In a particular embodiment, the crystal has the unit cell parameters a=242 Å±15 Å, b=71 Å±4 Å, c=72 Å±4 Å, the space group C₂ and forms a complex with BeF₃⁻.

It is appreciated that the ATPase is a bacterial type IB P-type ATPase.

In one embodiment the ATPase is a Legionella pneumophila type IB P-type ATPase. In another embodiment the ATPase is the LPG1024 (SEQ ID NO:1) Legionella pneumophila ATPase or an ATPase having at least 85% sequence identity with SEQ ID NO:1 or a functional homologue thereof.

The crystal of the present invention may in one embodiment comprise a type IB P-type ATPase, wherein said type IB P-type ATPase is a Cu⁺ ATPase.

In one embodiment the crystal according to the present invention effectively diffracts x-rays for the determination of the atomic structure of the protein to a resolution better than 3,7 Å such as better than 3,5 Å or such as better than 3 Å.

A second aspect of the present invention relates to a method for purification of a type IB P-type ATPase comprising the following steps:
a. obtaining a composition comprising a type IB P-type ATPase,
b. solubilising said type IB P-type ATPase using octaethylene glycol monododecyl ether (C₁₂E₈),
c. purifying said type IB P-type ATPase.

Another aspect relates to a method of growing a crystal comprising a type IB P-type ATPase according to claim 1, comprising the steps of:
a. obtaining a composition comprising a type IB P-type ATPase,
b. growing type IB P-type ATPase crystals in a crystallization environment including PEG and
c. obtaining crystals comprising a type IB P-type ATPase.

The method may further comprise a step of treating said composition comprising a type IB P-type ATPase with saturating amounts of one or more lipids before growing the type IB P-type ATPase crystals. In one preferred embodiment the type IB P-type ATPase is treated with dioleoyl-phosphatidylcholine before growing the crystals

Step b of the method relating to growing a crystal may further comprise growing type IB P-type ATPase crystals by vapour diffusion from hanging drops with a reservoir buffer containing PEG. The recevoir buffer may also be referred to as the precipitation buffer.

In one embodiment the concentration of PEG is 2-12% (w/v), such as 4-9% (w/v) or 5-7% (w/v). Further, the reservoir buffer may in one embodiment comprise 5-7% (w/v) PEG, 80-200 mM NaCl, 3% v/v t-BuOH and 5 mM BME.

In one preferred embodiment the PEG used is PEG 6000.

In another preferred embodiment step b of the method relating to growing a crystal may further comprise growing type IB P-type ATPase crystals by vapour diffusion from hanging drops with a reservoir buffer containing PEG 2000 MME

In a preferred embodiment the concentration of PEG 2000 MME is 2-20% (w/v), such as 6-16% (w/v) or 10-15% (w/v). The reservoir buffer may in one specific embodiment comprise 14% (w/v) PEG 2000 MME, 200 mM KCl, 3% (v/v) t-BuOH and 5 mM BME. In another preferred embodiment the recervoir buffer comprises 11% (w/v) PEG 2000 MME, 200 mM KCl and 5 mM BME.

A further aspect of the present invention relates to use of a crystal as described herein for determination of the three dimensional structure of said type IB P-type ATPase.

Another aspect of the present invention relates to use of a crystal as described herein for identifying inhibitors of a type IB P-type ATPase.

Use of the crystal for identifying inhibitors of a type IB P-type ATPase may further comprise a step of contacting the crystal with one or more compounds.

One aspect of the present invention relates to use of atomic coordinates as presented in Table 1 or atomic coordinates selected from a three-dimensional structure that deviates from the three-dimensional structure as presented in Table 1 by a root mean square deviation over protein backbone atoms of not more than 3 Å, in a method for identifying a inhibitor of a type IB P-type ATPase.

In another aspect the present invention relates to a method for identifying an inhibitor of a type IB P-type ATPase by determining binding interactions between the inhibitor and a set of binding interaction sites in said type IB P-type ATPase comprising the steps of:
a. generating the spatial structure of the type IB P-type ATPase on a computer screen using atomic coordinates as presented in Table 1 or atomic coordinates selected from a three-dimensional structure that deviates from the three-dimensional structure as presented in Table 1 by a root mean square deviation over protein backbone atoms of not more than 3 Å,
b. generating the spatial structure of inhibitors on the computer screen, and
c. selecting inhibitors that can bind to at least one amino acid residue of the set of binding interaction sites with out steric interference.

A further aspect of the invention relates to a computer-assisted method for identifying inhibitors of a type IB P-type ATPase using a programmed computer processor, a data storage system, a data input devise and a data output devise comprising the following steps:
a. putting into the programmed computer through said input device data comprising: a subset of the atoms of a type IB P-type ATPase, thereby generating a criteria data set, wherein the atomic coordinates are selected from the three-dimensional structure as presented in Table 1 or atomic coordinates selected from a three-dimensional structure that deviates from the three-dimensional structures as presented in Table 1 by a root mean square deviation over protein backbone atoms of not more than 3 Å,
b. comparing, using said processor, the criteria data set to a computer data base of low molecular weight organic chemical structures stored in the data storage system; and
c. selecting from said data base, using computer methods, a chemical structure having a portion that is structurally complementary to the criteria data set and being free of steric interference with the type IB P-type ATPase and/or
d. constructing using computer methods a model for a chemical structure having a portion that is structurally complementary to the criteria data set and being free of steric interference with the type IB P-type ATPase.

The criteria data set or the binding interaction sites may comprise one or more amino acid residues selected from the group comprising: Glu189, Met711, Met100 and Glu99 of SEQ ID NO:1.

The criteria data set or the binding interaction sites may comprise one or more amino acid residues selected from the group comprising: Cys382, Pro383, Cys384, Tyr688, Asn689, Met717 or Ser721 of SEQ ID NO:1.

The criteria data set or the binding interaction sites may also comprise one or more amino acid residues selected from the group comprising: Thr277, Gly278, Glu279, Asp426, Lys427, Thr428, Gly429, Thr430 and Thr432 of SEQ ID NO:1.

The criteria data set or the binding interaction sites may comprise one or more amino acid residues selected from the group comprising: Met148, Asp337 and Glu205 of SEQ ID NO:1.

The criteria data set or the binding interaction sites may also comprise one or more amino acid residues selected from the group comprising: Gly129 and Gly130 of SEQ ID NO:1.

Another aspect of the present invention relates to a method for identifying an inhibitor capable of inhibiting a type IB P-type ATPase, said method comprising the following steps:
a. identifying an inhibitor using atomic coordinates in conjunction with computer modelling, wherein said atomic coordinates are the atomic coordinates presented in Table 1 or wherein the atomic coordinates are selected from a three-dimensional structure that deviates from the three-dimensional structures presented in Table 1 by a root mean square deviation over protein backbone atoms of not more than 3 Å, by docking inhibitors into a set of binding interaction sites in a type IB P-type ATPase generated by computer modelling and selecting a inhibitor capable of binding to at least one amino acid in said type IB P-type ATPase,
b. providing said inhibitor and said type IB P-type ATPase,
c. contacting said inhibitor with said type IB P-type ATPase and
d. detecting inhibition the activity of said type IB P-type ATPase by the inhibitor.

It is appreciated that docking of inhibitor molecules is performed by employing the type IB P-type ATPase crystal defined by atomic coordinates presented in Table 1 and such that said inhibitor is capable of binding to at least three amino acid in the type IB P-type ATPase.

A further aspect relates to a method for identifying an inhibitor capable of inhibiting a type IB P-type ATPase, said method comprising the following steps:
a. introducing into a computer information derived from atomic coordinates presented in Table 1 or atomic coordinates selected from a three-dimensional structure that deviates from the three-dimensional structures as presented in Table 1 by a root mean square deviation over protein backbone atoms of not more than 3 Å,
b. generating a three-dimensional structure using said atomic coordinates,
c. superimposing a model of an inhibitor on said three-dimenssional structure;
d. assessing the possibility of binding and the absence of steric interference of the inhibitor with the type IB P-type ATPase;
e. incorporation said inhibitor in an activity assay of said type IB P-type ATPase and
f. determining whether said inhibitor inhibits the activity of said type IB P-type ATPase

In one embodiment information is derived from the atomic coordinates of at least one of the amino acid residues selected from the group comprising: Glu189, Met711, Met100 and Glu99 of SEQ ID NO:1.

In one embodiment information is derived from the atomic coordinates of at least one of the amino acid residues selected from the group comprising: Met148, Asp337 and Glu205 SEQ ID NO:1.

In a second embodiment information is derived from the atomic coordinates of at least one of the amino acid residues selected from the group comprising: Cys382, Pro383, Cys384, Tyr688, Asn689, Met717 and Ser721 SEQ ID NO:1.

In a third embodiment information is derived from the atomic coordinates of at least one of the amino acid residues selected from the group comprising: Thr277, Gly278, Glu279, Asp426, Lys427, Thr428, Gly429, Thr430 and Thr432 SEQ ID NO:1.

It is appreciated that the atomic coordinates are determined to a resolution of at least 3,5 Å.

The methods described herein may further comprise screening a library of small organic molecules and/or screening a library of peptide inhibitors.

Another aspect of the present invention relates to method for identifying a selective peptide inhibitor of a type IB P-type ATPase comprising the following steps:
a. identification of a inhibitor of a type IB P-type ATPase according to any of the claims,
b. contacting the peptide inhibitor with said type IB P-type ATPase,
c. contacting the peptide inhibitor with a different type IB P-type ATPase,
d. detecting inhibition of type IB P-type ATPase activity of said type IB P-type ATPase by the inhibitor and
e. detecting activity of said different type IB P-type ATPase in the presence of said inhibitor.

A further aspect relates to an inhibitor of a type IB P-type ATPase, wherein the inhibitor is identified according to the methods as described herein.

In one embodiment the inhibitor is capable of inhibiting growth of bacteria having type IB P-type ATPases in their cell membrane. The bacteria may in one embodiment be pathogenic bacteria.

An aspect of this invention relates to use of the inhibitor for treatment of an individual infected with pathogenic bacteria having type IB P-type ATPases in their cell membrane.

A further aspect relates to a method for treatment of an individual infected with pathogenic bacteria having type IB P-type ATPases in their cell membrane, said method comprising administering to said individual an inhibitor of a type IB P-type ATPase.

### DESCRIPTION OF DRAWINGS

FIG. 1: Architecture of the copper P-type ATPase CopA. A indicates actuator domain, N indicates nucleotide binding domain, P indicates phosphorylation domain and M indicates the domain comprising the transmembrane helices MA-B and M1-6. A, Unbiased electron density map used to build the model. The experimental map (grey) is calculated at 3.3 Å resolution and contoured at σ=1.0. The selenomethionine anomalous difference Fourier density map, contoured at σ=3.0 is shown. The refined model is shown as ribbon. B, Cartoon representation of the CopA structure. The electron density adjacent to the A-domain is believed to represent parts of the HMBD (for details see FIG. 3). The AlF₄ is bound to Asp426 at the domain A and P interface. Key residues mentioned in the text are shown as ball-and-sticks. Arrows indicate the vectorial transport of Cu⁺ and ATP consumption, while circles depict approximate Cu⁺ binding positions through the pump (transmembrane ion binding sites in black (Site Ito the left) while the entrance and exit sites are in grey).

FIG. 2: Details of the transmembrane domain of CopA as compared to SERCA1a. Transmembrane helices MA-B are shown in cyan, M1-6 in wheat and SERCA1a is displayed in green. The two proteins are aligned using M1-6 and with pdb-id 3b9r as the SERCA1a model. A, View from the cytosolic side. B, View from the cytosolic side of the membrane towards the M-domain. The transmembrane domain of CopA is relatively compact and its CPC motif is shifted about 4 Å towards M1-2 (and MA and MB). C, Side view of the transmembrane domain showing the residues associated with the ion binding sites of CopA and SERCA1a. Site I: Asn689 (M5), Met717 (M6) and Ser721 in CopA; and Glu771 (M5), Asp800 (M6) and Glu908 (M8) in SERCA1a. Site II: Cys382 and Cys384 (CPC motif of M4) as well as Tyr688 (M5) in CopA and IIe307 and Glu309 (IPE motif in M4) as well as Asp800 (M6) in SERCA1a. All residues are depicted as sticks.

FIG. 3: The position of the heavy metal binding domain. Unbiased electron density contoured at σ=1.0 (cyan) adjacent to the linker regions of the A-domain (yellow) is assigned to a disordered position of the heavy metal binding domain. The electron density was computed at 3.3 Å resolution using experimental MIRAS phases improved by density modification (without solvent mask) in Resolve. The anomalous difference Fourier map calculated from selenomethionine crystals is contoured at σ=3.0 in orange and from p-Chloromercuric benzoic acid soaked crystals at σ=6.0 in red. The first modeled residue, Va174, is positioned about 15-20 Å away from the HMBD electron density. For the equivalent Fig. with SERCA1a aligned, see FIG. 12. For comparison to the HMBD size, see FIG. 11.

FIG. 4: The platform with a funnel leading to the M4 CPC motif. The domains are colored as in FIG. 1. A, Details of the platform, including the kink of transmembrane helix MB and the following amphiphatic helix, located approximately at the membrane interface. The kink is induced by the conserved Gly129-Gly130 motif and hydrophobic side chains are directed towards the membrane and hydrophilic residues face the cytoplasm (residues shown as sticks). B, View from the cytoplasmic side towards the CPC motif implicated in copper binding in the transmembrane region. The GG kink motif, the suggested pre-coordination site (Met148, Glu205 and Asp337) as well as the side chains assigned to the high-affinity Cu⁺ sites (see FIG. 2C) in the membrane are displayed as sticks.

FIG. 5: The CopA copper transport mechanism. A, Schematics of the reaction cycle of CopA. Simple models of the conformational states (except for the E2-P* structure) were generated by structural alignment to the corresponding SERCA1 structures. The CopA domains of the background are colored in black and white. Proposed movements of transmembrane helices are indicated by arrows. Key residues for copper transport (Met148 of M1, Cys382 and Cys384 of M4, Met717 and Ser721 of M6 and Asp337 above M1) are illustrated as sticks. Copper ions are shown as spheres and the copper pathway by arrows. The suggested mechanism involves copper binding to a pre-coordination site (including Met148) adjacent to the GG kink motif and the membrane interface membrane. With the conformational changes that occur from the E2 to the E1 state, this pre-coordination site is destroyed and the copper can be transferred by Met148 to Cys382 of the CPC motif. For Cu⁺ release, the opposite conformational changes (redirecting Cys382 towards Met148) will significantly decrease the copper affinity and allow the ions to be extruded. B, Close view displaying Cys382 and the neighboring amino acids in the structurally determined E2 state indicating how the preceding high-affinity copper-bound conformations are destroyed. Note that Leu151 and IIe152 interact with the sulfur of Cys382 and that and a large side chain at position Gly155 would prevent Cys382 from obtaining its current orientation (the 155 position is conserved as a glycine or alanine). Final 2Fₒ-F_{c} electron-densities are contoured at σ=1.0.

FIG. 6: Distribution of ATP7A missense mutations associated with Menkes disease mapped on the L. pneumophila CopA structure. LpCopA is colored in white and the affected residues shown as spheres. References for previously determined mutations can be found in Table 9 and data covering the mutations identified in this work in Table 10. Some mutations are not covered by the LpCopA structure and are not shown (Ala629Pro, Lys633Arg, Met687Val, Glu628Val and Pro1413Arg). A, The distribution of mutations according to function and structural position. Underscored amino acids are identical between LpCopA and human ATP7A and residue numbers refer to human ATP7A. B, The distribution of phenotypic severity of Menkes mutations. The disease phenotype ranges from classical to relatively mild “distal hereditary motor neuropathy” (dHMN). The phenotype is unknown for Ser657Arg. Labels indicate the cellular localization of the mutant proteins when known: No=no detectable protein in cells; TGN=trans-Golgi-network; pTGN=partly located in TGN.

FIG. 7: Similarity of P-type ATPases. A, Sequence alignment between full-length CopA proteins (all containing the CPC motif in M4, YN in M5, and MXXSS in M6-motifs associated with Cu⁺/Ag⁺ pumps). Searching for the latter two motifs among 3260 P-type ATPases with the CPC motif we identified 1713 full-length sequences (average length 836 and maximum length 1683) which were aligned with MUSCLE (Kuhlbrandt, W., Nat Rev Mol Cell Biol 5 (4), 282 (2004)). This subset of P-type ATPAses includes LpCopA (Q8RNP6), human ATP7A (Menkes, Q04656) and ATP7B (Wilson, P35670). The Fig. shows these three protein sequences with the consensus sequence from the 1713 sequences aligned, as generated by the program Jalview (insertions in the consensus sequence relative to the mentioned sequences have been removed). The background colors indicate the sequence conservation among all 1713 sequences colored from black (highly conserved) to white (medium conservation) to dark grey (poor conservation). The pattern of sequence conservation extracted from these 1713 sequences was similar to an alignment procedure based on 17 type IB P-type ATPases with confirmed copper specificity. The approximate position of the transmembrane helices and the soluble domains are displayed above the alignment. Asterisks under the alignment indicate residues discussed in the text/examples. B, Structural alignment of all P-type ATPases of known structure including rabbit SERCA1a (B6CAM1), pig renal Na⁺,K⁺-ATPase (P05024), Arabidopsis thaliana H⁺-ATPase (P19456) and LpCopA (Q8RNP6). The alignment was performed with the software Pymol by aligning the structures on the corresponding state of SERCA1a. Asterisks under the alignment indicate residues discussed in the text. C, The multiple sequence alignment from A plotted on the structure of LpCopA using the ConSurf server (http://consurf.tau.ac.il/). The colors are the same as in A.

FIG. 8: Cu(I) induced ATPase activity for LpCopA complexed with AlF₄⁻. All experiments were conducted in 1 mg/mL E. coli total lipid extract and 20 mM cysteine without which it is difficult to monitor any significant ATPase activity for CopA proteins. No LpCopA activity was observed in the absence of these two, neither for Cu²⁺/Zn²⁺ nor Cu⁺/Ag⁺, Cysteine can be substituted by reduced glutathione (GSH) but not oxidised glutathione (data not shown). As cysteine (or GSH) will rapidly reduce Cu²⁺ to Cu⁺, Cu⁺ predominates in all such conditions with copper⁴. The monitored absorbance indicates the amount of inorganic phosphate generated by ATP hydrolysis. A, Measured LpCopA activity in the presence of different ions (displayed as the relative activity when the copper-chelated background (by TTM) has been subtracted). Variations in the reaction buffer, from left to right: No extra supplementation; 10 μM TTM; 100 μM CuCl₂; 100 μM CuCl₂ (from a stock solution of 20 mM ascorbic acid and 10 mM CuCl₂); 100 μM AgNo₃; 100 μM ZnCl₂. Cu⁺ and Ag⁺ clearly stimulate ATPase activity. B, Enzymatic activity of LpCopA and HMBD-truncated LpCopA in the presence of increasing amounts of Cu⁺ (subtracting the TTM background) indicates an enzymatic induced process Variations in the reaction buffer, from left to right: No extra supplementation; 10 μM TTM; increasing concentration of CuCl₂ (from 10 nM to 10 mM CuCl₂ including 10 μM TTM at all assessed copper concentrations). Highest activity is observed at 1 mM CuCl₂ for the full-length LpCopA above which it appears to be inhibited. For the truncated construct, the optimum was observed at about 1 mM CuCl₂. Three times more protein of the HMBD-truncated construct was employed in these activity studies as compared to the full-length protein. Combined with the published complementation by LpCopA using a CopA deleted E. coli strain (through copper extrusion from the reduced intracellular environment where Cu²⁺ is rare) these data clearly indicate that LpCopA pumps Cu⁺.

FIG. 9: Surface charge distribution of LpCopA complexed with AlF₄⁻. The cytoplasmic membrane interface is clearly marked in LpCopA by the “positive inside” rule (ref). The charge distribution was generated using the APBS plugin to Pymol (the potential on the solvent accessible surface is indicated by the contrast −6/6). Black arrows indicate the position of the amphipathic and C-terminal fraction of MB.

FIG. 10: The phosphorylation site of the LpCopA complexed with AlF₄⁻ structure. The domains are indicated by colors as in FIG. 1. The AlF₄ and the Mg²⁺ ion are associated with Asp426 (in the DKTGT motif of the P-domain) at the interface between the A and P domains. Asp426, Thr428, Thr577, Asp624, Asn627 and Asp628 (in the P-domain) as well as Thr277, Gly278 and Glu279 (the TGE motif in the P-domain) are shown as sticks.

FIG. 11: A structure of the heavy metal binding domain placed at the observed electron density for the domain. The orientation and colors are equivalent to FIG. 3.

The heavy metal binding domain (white ribbon) is based on the third domain of human ATP7A (pdb-id 2g9o) and it has been placed next to the residual electron density only to illustrate their relative sizes (i.e. not fitted to the density). See FIG. 3 and FIG. 12 for comparison.

FIG. 12: The position of observed electron density ascribed to the heavy metal binding domain coincides with the location for the N-terminal fraction of the A-domain in SERCA1a. The FIG. 12 is equivalent to FIG. 3 with SERCA1a (green, pdb-id 3b9r) superimposed on LpCopA (Ca atoms). For comparison to the HMBD size, see FIG. 11.

FIG. 13: The surface conservation of CopA proteins. Surface conservation of LpCopA depicted from black (highly conserved) to white to dark grey (poorly conserved) using the alignment presented in FIG. 7A. The surface conservation is shown from four different side-view orientations as well as from the extra- and intra-cellular sides, respectively. SERCA1a displays the same type of surface conservation (data not shown). For comparison, the unbiased electron density assigned to the HMBD (indicated as in FIGS. 1 and 3) is shown in cyan.

FIG. 14: A putative copper exit pathway in CopA. Close view displaying Glu189 and neighboring residues in the LpCopA structure (colors as in FIG. 1). The final 2Fₒ-F_{c} and Fₒ-F_{c} electron-density maps are contoured at σ=1.0 (blue) and σ=4.0 (purple), respectively. Possibly, a cation from the crystallization medium (modeled as present at 40 mM concentration in the crystal interacts with the highly conserved Glu189 and Met100 and the less conserved Glu99 and Met711, not far from Met717 which is a proposed ligand for ion binding site I (all shown in sticks). The distance from Met717 to Glu189 is 3.7 Å (side-chain to side-chain).

FIG. 15: Crystal packing in the LpCopA type I crystals complexed with AlF₄⁻. The crystal packing is shown in two perpendicular orientations, and the CopA domains are colored as in FIG. 1. The proteins are arranged as if in stacked bilayers, held together by hydrophobic interactions between their membrane-spanning regions. The crystal form displays up-and-down arrangements of monomers with different bilayers interacting by head-to-head interactions of the soluble headpieces (cytoplasmic domains). The unit cell of the crystal (P 1 space group) is indicated by black rectangles. The unit cell parameters are a=44.1 Å, b=72.9 Å, c=329.6 Å, α=89.97 °, β=90.04°, γ=90.22°.

FIG. 16: Details of the E₂P LpCopA structure complex with BeF₃⁻. The overall CopA BeF₃⁻ structure is superimposed on the backbone of the P-domain of the previously determined LpCopA-AlF₄⁻ (orange). A main movement can be seen in the A- and N-domains and slight movements in TM 2, TM A and B (A). The binding site of the P-domain includes BeF₃⁻ on Asp 426 in the DKTG motif and it contains a coordinated Mg₂⁺. The TGE motif is shown without Gly 278 and the DKTG motif without Gly 429. The loop containing the TGE motif from LpCopA-AlF₄⁻ moves upon phosphate release (orange). (B). The simulated annealing omit map (yellow-orange) confirms the presence of β-NAD. Atoms of β-NAD after the β-phosphate show no clear density features and have been set to 0 occupancy. Hydrogen bonds indicate coordination of β-NAD to specific residues in the N domain as well as waters (C).

FIG. 17: Crystal packing of the E2P LpCopA structure complex with BeF₃⁻ and SERCA1a. Much tighter membrane packing is observed in the LpCopA structure (A, left) than in SERCA1a (B, left). The cytoplasmic domains in LpCopA (A, right) pack similar as in SERCA1a (B, right). SERCA1a PDB code: 3B9B

FIG. 18: Waters in the transmembrane domain of E₂P CopA. A simulated annealing omit map resolves density for different waters in the transmembrane domain of LpCopA. Amino acids that interact with these waters as well as the amino acids for Cu (I) pathway are shown

FIG. 19: Fo-Fo isomorphous difference map of LpCopA complexed with AlF₄⁻ and MgF₄²⁻ respectively. The transmembrane domain (A) and the cytoplasmic domains (B, AlF₄⁻; C, MgF₄²⁻) indicate no differences between the two dephosphorylation analogs. Map is plotted at 3.5 σ, the red density represents the observed structure factor of MgF₄²⁻ state and green density represents the observed structure factor of AlF₄⁻. Lower sigmalevels showed more noise.

### DETAILED DESCRIPTION OF THE INVENTION

### Definitions

The term “crystal” as used herein refers to a homogenous solid formed by a repeating, three-dimensional pattern of atoms, ions or molecules and having fixed distances between constituent parts. In the present invention the term “crystal” refers to a protein crystal. Proteins are crystallized from generally complex solutions that may include not only the target molecule but also buffers, salts, precipitating agents, water and any number of small binding proteins. It is important to note that protein crystals are composed not only of protein, but also of a large percentage of solvents molecules, in particular water. These contents may vary from 30% to even 90%. Protein crystals may accumulate greater quantities and a diverse range of impurities which cannot be listed here or anticipated in detail. The skilled person knows that some crystals diffract better than others. Crystals vary in size from a barely observable 20 micron to 1 or more millimetres. Crystals useful for X-ray analysis are typically single, 0.05 millimetres or larger, and free of cracks and other defects.

The term “coordinate” or “atom coordinate” as used herein, refers to the atomic arrangement of the crystal, and the atom coordinates define the crystal structure. The final map containing the atomic coordinates of the constituents of the crystal may be stored on a data carrier; typically the data is stored in PDB or CIF format which are known to the person skilled in the art. The PDB and CIF formats are organized according to the instructions and guidelines given by the Research Collaboratory for Structural Bioinformatics.

The term “unit cell” as used herein refers to the simplest repeating unit in a crystal. The unit cell is characterised in having three edge lengths a, b and c and three internal angels α, β and γ. The edge lengths a, b and c are also referred to as the unit cell parameters.

The term “root mean square deviation” (rmsd) is used as a mean of comparing two closely related structures and relates to a deviation in the distance between related atoms of the two structures after structurally minimizing this distance in an alignment. Related proteins with closely related structures are characterized by relatively low RMSD values whereas more changes results in an increase of the RMSD value. The term “associating with” or “binding” refers to a condition of proximity between chemical entities or compounds, or portions thereof. The association may be noncovalent, wherein the juxtaposition is energetically favoured by hydrogen bonding or van der Waals or electrostatic interactions- or it may be covalent.

The term “binding pocket”, as used herein, refers to a region of a molecule or molecular complex that, as a result of its shape, favourably associates with another molecule, molecular complex, chemical entity or compound. As used herein, the pocket comprises at least a deep cavity and, optionally a shallow cavity.

The terms “transfer” or “transport” as used herein refers to the “transfer” or “transport” of ions across the cell membrane. The “transfer” or “transport” is mediated by P-type ATP-ases.

As used herein the term “complex” refers to the combination of a molecule or a protein, conservative analogues or truncations thereof associated with a chemical entity.

As used herein the term “Cu⁺ transport pathway” refers to a pathway that results in the transport Cu⁺ across the cell membrane. The transport of Cu⁺ across the cell membrane is mediated by a type IB P-type ATPase.

The term “inhibitor” as used herein refers to a compound or peptide that is capable of inhibiting or reducing the activity of an enzyme. The inhibitors described herein are capable of inhibiting or reducing the activity of a type IB P-type ATPase. The inhibitor may be able to reduce the activity of the ATPase activity of the type IB P-type ATPase and/or the metal ion transporting activity. In one embodiment the inhibitor reduces or inhibits the activity, such as the ATPase activity and/or the Cu⁺ transporting activity, of a Cu⁺ ATPase. The term “inhibitor” may herein be used interchangeably with the term “modulator”.

The term “extracellular site” as used herein refers to the site of the cell membrane, which is the outer site of the cell.

The term “intracellular site” as used herein refers to the site of the cell membrane, which is the inner site of the cell membrane facing the cytosol.

The term “cytosol” as used herein refers to the intracellular fluid, which is the liquid found inside the cell but outside the nucleus and cellular organelles such as for example mitochondria.

Amino acid: Entity comprising an amino terminal part (NH₂) and a carboxy terminal part (COOH) separated by a central part comprising a carbon atom, or a chain of carbon atoms, comprising at least one side chain or functional group. NH₂ refers to the amino group present at the amino terminal end of an amino acid or peptide, and COOH refers to the carboxy group present at the carboxy terminal end of an amino acid or peptide.

The generic term amino acid comprises both natural and non-natural amino acids. Natural amino acids of standard nomenclature as listed in J. Biol. Chem., 243:3552-59 (1969) and adopted in 37 C.F.R., section 1.822(b)(2) belong to the group of amino acids listed in Table 14 herein below. Non-natural amino acids are those not listed in Table 14. Examples of non-natural amino acids are those listed e.g. in 37 C.F.R. section 1.822(b)(4), all of which are incorporated herein by reference. Further examples of non-natural amino acids are listed herein below. Amino acid residues described herein can be in the “D” or “L” isomeric form.

TABLE 14
Natural amino acids and their respective codes:

| Symbols | | |
|---|---|---|
| 1-Letter | 3-Letter | Amino acid |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

The term Angstrom (Å) is a unit of length equal to 0.1 nanometer or 1×10⁻¹⁰ meters.

### Type IB P-Type ATPase Crystals

One aspect of the present invention relates to a crystal comprising a type IB P-type ATPase, wherein said crystal is characterised in having the space group P1.

In order to stabilize the protein one or more compounds may be added during purification of the ATPase (see below) enabling formation of an ATPase complex suited for crystallization. This may further enable fixing of the protein in a specific state which is needed to obtain detailed information regarding the functionality of the ATPase.

According to the invention the crystals may comprise one or more compounds for stabilising the protein, such as ATP, ATP analogues (such as AMPPCP), or ADP or ADP analogue, or other nucleotide analogues for which the ATPase has suitable affinity for use in structural determination. Such analogues may provide stability by fixing the protein in a specific state. In an embodiment the crystal comprises a nonhydrolysable ATP analogue preferably AMPPCP.

The type IB P-type ATPase may form a complex with compounds containing fluoride. In one embodiment of the present invention the type IB P-type ATPase forms a complex with AlF₄⁻. In a particular embodiment the type IB P-type ATPase in complex with AlF₄⁻ is in an E₂-P transition state.

In another preferred embodiment the type IB P-type ATPase forms a complex with MgF₄²⁻. In yet another preferred embodiment the type IB P-type ATPase forms a complex with BeF₃⁻. In a particular embodiment the type IB P-type ATPase in complex with BeF₃⁻ or MgF₄²⁻ is in an E₂-P transition state.

For various purposes different cations may be included in the crystal. Such cations may be included in the crystal by growing the crystal in the presence of said cations or by submerging the crystals in a solution comprising cations. Heavy atoms that bind to the protein are frequently included in protein structure determination projects to obtain phase information.

The crystal structures may comprise cations selected from the group comprising: Pt⁴⁺, Hg²⁺, Ir³⁺ and Ta²⁺. The crystal may further comprise amino acid derivatives or seleno-methionine.

In one embodiment the type IB P-type ATPase forms a complex with AlF₄⁻, wherein AlF₄⁻ is coordinated by Mg²⁺. Thus, the crystal of the present invention may comprise Mg²⁺.

The crystal structures may according to the invention further comprise remains from the buffer composition used during the crystallisation process, such as one or more compounds selected from the group of poly ethylene glycols (PEGs) comprising: PEG 100, PEG 200, PEG 400, PEG 600, PEG 800, PEG 1000, PEG 2000, PEG 3000, PEG 4000, PEG 5000, PEG 6000, PEG 7000, PEG 8000, PEG10000, PEG15000 and PEG 20000. Likewise PMEs and/or MMEs may be used.

HEPES, Mes, and MOPS are further standard buffers, which according to the invention can be comprised by the crystal.

The crystals may further comprise one or more compounds selected from the group of salts ions comprising the cations and anions Mg, Ca, Na, Cl, Br, I, Rb, P, S, K, Mn, Zn, Cu, B, Mo, Se, Si and Co.

Preferably the crystal comprises KCl, MOPS and one of the PEG compounds as mentioned above. Preferably PEG 6000 or PEG 2000 MME is used.

### Unit Cell Parameters

In a first embodiment the crystal is characterised in having unit cell parameters a=44 Å±8 Å, b=73 Å±8 Å, c=330 Å±8 Å, such as a=44 Å±7 Å, b=73 Å±7 Å, c=330 Å±7 Å, such as for example a=44 Å±6 Å, b=73 Å±6 Å, c=330 Å±6 Å, such as a=44 Å±5 Å, b=73 Å±5 Å, c=330 Å±5 Å, such as for example a=44 Å±3 Å, b=73 Å±3 Å, c=330 Å±3 Å, such as a=44 Å±2 Å, b=73 Å±2 Å, c=330 Å±2 Å, such as for example a=44 Å±1 Å, b=73 Å±1 Å, c=330 Å±1 Å.

In one embodiment the crystal is characterised in having unit cell parameters a=44 Å±8 Å, b=73 Å±10 Å, c=330 Å±50 Å, such as a=44 Å±7 Å, b=73 Å±9 Å, c=330 Å±45 Å, such as for example a=44 Å±6 Å, b=73 Å±8 Å, c=330 Å±40 Å, such as a=44 Å±5 Å, b=73 Å±6 Å, c=330 Å±30 Å, such as for example a=44 Å±4 Å, b=73 Å±5 Å, c=330 Å±25 Å, such as a=44 Å±2 Å, b=73 Å±3 Å, c=330 Å±2 Å, such as for example a=44 Å±1 Å, b=73 Å±1 Å, c=330 Å±5 Å.

In another embodiment the crystal is characterised in having cell parameters a=39 Å-49 Å, b=68 Å-78 Å, c=325 Å-335 Å, such as for example a=40 Å-48 Å, b=69 Å-77 Å, c=326 Å-334 Å, such as a=41 Å-47 Å, b=70 Å-76 Å, c=327 Å-333 Å, such as for example a=42 Å-46 Å, b=71 Å-75 Å, c=328 Å-332 Å, such as a=43 Å-45 Å, b=72 Å-74 Å, c=229 Å-331 Å.

In one preferred embodiment the crystal is characterised in having unit cell parameters a=44 Å±4 Å, b=73 Å±4 Å, c=330 Å±4 Å. it is preferred that the crystal having the unit cell parameters a=44 Å±4 Å, b=73 Å±4 Å, c=330 Å±4 Å forms a complex with AlF₄⁻.

In another preferred embodiment the crystal is characterised in having unit cell parameters a=44 Å±3 Å, b=73 Å±4 Å, c=330 Å±20 Å. It is preferred that the crystal having the unit cell parameters a=44 Å±3 Å, b=80 Å±4 Å, c=330 Å±20 Å forms a complex with AlF₄⁻.

In a particular preferred embodiment the crystal is characterised in having unit cell parameters a=44.1 Å, b=72.9 Å and c=329.6 Å. It is preferred that the crystal having the unit cell parameters a=44.1 Å, b=72.9 Å and c=329.6 Å forms a complex with AlF₄⁻.

In a second embodiment the crystal is characterised in having unit cell parameters a=44 Å±8 Å, b=73 Å±8 Å, c=329 Å±8 Å, such as a=44 Å±7 Å, b=73 Å±7 Å, c=329 Å±7 Å, such as for example a=44 Å±6 Å, b=73 Å±6 Å, c=329 Å±6 Å, such as a=44 Å±5 Å, b=73 Å±5 Å, c=329 Å±5 Å, such as for example a=44 Å±3 Å, b=73 Å±3 Å, c=329 Å±3 Å, such as a=44 Å±2 Å, b=73 Å±2 Å, c=329 Å±2 Å, such as for example a=44 Å±1 Å, b=73 Å±1 Å, c=329 Å±1 Å.

In another embodiment the crystal is characterised in having unit cell parameters a=44 Å±8 Å, b=73 Å±10 Å, c=329 Å±50 Å, such as a=44 Å±7 Å, b=73 Å±9 Å, c=329 Å±45 Å, such as for example a=44 Å±6 Å, b=73 Å±8 Å, c=329 Å±40 Å, such as a=44 Å±5 Å, b=73 Å±6 Å, c=329 Å±30 Å, such as for example a=44 Å±4 Å, b=73 Å±5 Å, c=329 Å±25 Å, such as a=44 Å±2 Å, b=73 Å±3 Å, c=329 Å±2 Å, such as for example a=44 Å±1 Å, b=73 Å±1 Å, c=329 Å±5 Å.

In yet another embodiment the crystal is characterised in having cell parameters a=39 Å-49 Å, b=68 Å-78 Å, c=324 Å-334 Å, such as for example a=40 Å-48 Å, b=69 Å-77 Å, c=325 Å-333 Å, such as a=41 Å-47 Å, b=70 Å-76 Å, c=326 Å-332 Å, such as for example a=42 Å-46 Å, b=71 Å-75 Å, c=327 Å-331 Å, such as a=43 Å-45 Å, b=72 Å-74 Å, c=228 Å-330 Å.

In one preferred embodiment the crystal is characterised in having unit cell parameters a=44 Å±4 Å, b=73 Å±4 Å, c=330 Å±4 Å. It is preferred that the crystal having the unit cell parameters a=44 Å±4 Å, b=73 Å±4 Å, c=330 Å±4 Å forms a complex with MgF₄²⁻.

In another preferred embodiment the crystal is characterised in having unit cell parameters a=44 Å±3 Å, b=73 Å±4 Å, c=330 Å±20 Å. It is preferred that the crystal having the unit cell parameters a=44 Å±3 Å, b=73 Å±4 Å, c=329 Å±20 Å forms a complex with MgF₄²⁻.

In a particular preferred embodiment the crystal is characterised in having unit cell parameters a=44 Å, b=73 Å and c=329 Å. It is preferred that the crystal having the unit cell parameters a=44 Å, b=73 Å and c=329 Å forms a complex with MgF₄²⁻.

In a third embodiment the crystal is characterised in having unit cell parameters a=242 Å±8 Å, b=71 Å±8 Å, c=72 Å±8 Å, such as a=242 Å±7 Å, b=71 Å±7 Å, c=72 Å±7 Å, such as for example a=242 Å±6 Å, b=71 Å±6 Å, c=72 Å±6 Å, such as a=242 Å±5 Å, b=71 Å±5 Å, c=72 Å±5 Å, such as for example a=242 Å±3 Å, b=71 Å±3 Å, c=72 Å±3 Å, such as a=242 Å±2 Å, b=71 Å±2 Å, c=72 Å±2 Å, such as for example a=242 Å±1 Å, b=71 Å±1 Å, c=72 Å±1 Å.

In another embodiment the crystal is characterised in having unit cell parameters a=242 Å±40 Å, b=71 Å±10 Å, c=72 Å±10 Å, such as a=242 Å±35 Å, b=71 Å±8 Å, c=72A±8 Å, such as for example a=242 Å±30 Å, b=71 Å±7 Å, c=72 Å±7 Å, such as a=242 Å±25 Å, b=71 Å±6 Å, c=72 Å±6 Å, such as for example a=242 Å±20 Å, b=71 Å±5 Å, c=72 Å±5 Å, such as a=242 Å±10 Å, b=71 Å±c=72 Å±3 Å, such as for example a=242 Å±5 Å, b=71 Å±2 Å, c=72 Å±2 Å.

In yet another embodiment the crystal is characterised in having cell parameters a=237 Å-247 Å, b=66 Å-76 Å, c≈67 Å-77 Å, such as for example a=238 Å-246 Å, b=67A-75 Å, c=68 Å-76 Å, such as a=239 Å-245 Å, b=68 Å-74 Å, c=69 Å-75 Å, such as for example a=240 Å-244 Å, b≈69 Å-73 Å, c=70 Å-74 Å, such as a=241 Å-243 Å, b=70 Å-72 Å, c=71 Å-73 Å.

In one preferred embodiment the crystal is characterised in having unit cell parameters a=242 Å±4 Å, b=71 Å±4 Å, c≈72 Å±4 Å. It is preferred that the crystal having the unit cell parameters a=242 Å±4 Å, b=71 Å±4 Å, c=72 Å±4 Å forms a complex with BeF₃⁻.

In a particular embodiment, the crystal has the unit cell parameters a=242 Å±15 Å, b=71 Å±4 Å, c=72 Å±4 Å. It is preferred that the crystal having the unit cell parameters a=242 Å±15 Å, b=71 Å±4 Å, c=72 Å±4 Å forms a complex with BeF₃⁻.

In a particular preferred embodiment the crystal is characterised in having unit cell parameters a=242 Å, b=71 Å and c=72 Å. It is preferred that the crystal having the unit cell parameters a=242 Å, b=71 Å and c=72 Å forms a complex with BeF₃⁻.

### Angels

In a first embodiment the crystal is characterised in having the angels α=89.8°-90.0°, β=89.9°-90.1° and γ=90.1°-90.3°. In a preferred ambodiment the crystal has the angels α=89.8°-90.0°, β89.9°-90.1° and γ=90.1°-90.3° and forms a complex with AlF₄⁻. In a particular embodiment the crystal forms a complex with AlF₄⁻, is characterised in having the unit cell parameters a=44 Å±3 Å, b=80 Å±4 Å, c=330 Å±20 Å and/or the angels α=89.8°-90.0°, β=89.9°-90.1° and γ=90.1°-90.3°.

In a particular embodiment the crystal is characterized in having the angels α=89.97°, β=90.04° and γ=90.22°. In a preferred ambodiment the crystal has the angels α=89.97°, β=90.04° and γ=90.22° and forms a complex with AlF₄⁻. In a particular embodiment the crystal forms a complex with AlF₄⁻, is characterised in having the unit cell parameters a=44.1 Å, b=72.9 Å and c=329.6 Å and/or the angels α=89.97°, β=90.04° and γ=90.22°.

In a second embodiment the crystal is characterised in having the angels α=89.9°-90.1°, β=89.9°-90.1° and γ=89.9°-90.1° In a preferred ambodiment the crystal has the angels α=89.9°-90.1°, β≈89.9°-90.1° and γ=89.9°-90.1° and forms a complex with MgF₄²⁻. In a particular embodiment the crystal forms a complex with MgF₄²⁻, is characterised in having the unit cell parameters a=44 Å±3 Å, b=73 Å±4 Å, c=329 Å±20 Å and/or the angels α=89.9°-90.1°, β=89.9°-90.1° and γ=89.9°-90.1°.

In a particular embodiment the crystal is characterized in having the angels α=90.00°, β=90.00° and γ≈90.00°. In a preferred ambodiment the crystal has the angels α=90.00°, β=90.00° and γ=90.00° and forms a complex with MgF₄²⁻. In a particular embodiment the crystal forms a complex with MgF₄²⁻, is characterised in having the unit cell parameters a=44 Å, b=73 Å and c=329 Å and/or the angels α=90.00°, β=90.00° and γ=90.00°.

In a third embodiment the crystal is characterised in having the angels α=89.9°-90.1°, β=100.00°-100.02° and γ=89.9°-90.1°. In a preferred ambodiment the crystal has the angels α=89.9°-90.1°, β=100.00°-100.02° and γ=89.9°-90.1° and forms a complex with BeF₃⁻. In a particular embodiment the crystal forms a complex with BeF₃⁻, is characterised in having the unit cell parameters a=242 Å±15 Å, b=71 Å±4 Å, c=72 Å±4 Å and/or the angels α=89.9°-90.1°, β=100.00°-100.02° and γ=89.9°-90.1°.

In a particular embodiment the crystal is characterized in having the angels α=90.00°, β=100.01° and γ=90.00°. In a preferred ambodiment the crystal has the angels α=90.00°, β=100.01° and γ=90.00° and forms a complex with BeF₃⁻. In a particular embodiment the crystal forms a complex with BeF₃⁻, is characterised in having the unit cell parameters a=242 Å, b=71 Å and c=72 Å and/or the angels α=90.00°, β=100.01° and γ=90.00°.

### Space Group

In one embodiment the crystal is characterised in having the space group P2₁2₁2₁. The space group P2₁2₁2₁ is also referred to as the pseudo-symmetry of the crystal. The space group P2₁2₁2₁ is a subgroup of the space group P1.

In one embodiment the crystal is characterised in having four protein monomers in the unit cell related by P2₁2₁2₁ pseudo-symmetry. In a preferred embodiment the crystal is characterised in having the space group P2₁2₁2₁ and forms a complex with AlF₄⁻. In a preferred embodiment the crystal is characterised in having the space group P2₁2₁2₁ and/or forms a complex with MgF4^{2-.}

In a first embodiment the crystal is characterised in having the space group P1 with the unit cell parameters a=44 Å±3 Å, b=80 Å±4 Å, c=330 Å±20 Å and/or in having the space group P2₁2₁2₁. In a particular embodiment, the crystal has the unit cell parameters a=44 Å±3 Å, b=80 Å±4 Å, c=330 Å±20 Å, the space group P2₁2₁2₁ and/or forms a complex with AlF₄⁻. In another particular embodiment, the crystal forms a complex with AlF₄⁻, is characterised in having the unit cell parameters a=44 Å±3 Å, b=80 Å±4 Å, c=330 Å±20 Å, the angels α=89.8°-90.0°, β=89.9°-90.1°, γ=90.1°-90.3° and/or the space group P2₁2₁2₁.

In a second embodiment the crystal is characterised in having the space group P1 with the unit cell parameters a=44 Å±3 Å, b=73 Å±4 Å, c=329 Å±20 Å and/or in having the space group P2₁2₁2₁. In a particular embodiment, the crystal has the unit cell parameters a=44 Å±3 Å, b=73 Å±4 Å, c=329 Å±20 Å, the space group P2₁2₁2₁ and/or forms a complex with MgF₄²⁻. In another particular embodiment, the crystal forms a complex with MgF₄²⁻, is characterised in having the unit cell parameters a=44 Å±3 Å, b=73 Å±4 Å, c=329 Å±20 Å, the angels α=89.9°-90.1°, β=89.9°-90.1°, γ89.9°-90.1° and/or the space group P2₁2₁2₁.

In a third embodiment the crystal is characterised in having the space group P1 with the unit cell parameters a=242 Å±15 Å, b=71 Å±4 Å, c=72 Å±4 Å and/or in having the space group C₂. C₂ is also referred to as the pseudo-symmetry of the crystal. In a preferred embodiment, the crystal having the space group C₂ forms a complex with BeF₃⁻. In a particular embodiment, the crystal has the unit cell parameters a=242 Å±15 Å, b=71 Å±4 Å, c=72 Å±4 Å, the space group C₂ and/or forms a complex with BeF₃⁻. In another particular embodiment, the crystal forms a complex with BeF₃⁻, is characterised in having the unit cell parameters a=242 Å±15 Å, b=71 Å±4 Å, c=72 Å±4 Å, the angels α=89.9°-90.1°, β=100.00°-100.02° and γ=89.9°-90.1° and/or the space group C₂.

The cell dimensions can according to the application vary depending on the specific ATPase comprised by the crystal an even on the conformation of the ATPase comprised by the crystal.

Depending on the resolution of a crystal structures different information can be obtained from the data. At a resolution of about 6 Å the overall shape of molecular parts is resolved, such as a-helices that are seen as rods with strong intensity. At a resolution of about 3.5 Å the main chain is visible (usually with some ambiguities). At a resolution of about 3 Å the side chains are partly resolved. At a resolution of about 2.5 Å the side chains are well resolved. The atoms are located within about 0.4 Å meaning that the lengths of hydrogen bonds calculated from a PDB file (for example, by RasMol) have at least this uncertainty. The normal limit of protein crystallography is around 1 Å or slightly less, where atoms are located at below ±0.1 Å.

The crystal of the invention preferably effectively diffracts x-rays for the determination of the atomic structure of the protein to a resolution better than 6 Å. More preferably the three dimensional structure determinations can be determined with a resolution better than 5 Å, such as better than 4 Å or better using the crystals according to the invention. In one preferred embodiment the effectively diffracts x-rays for the determination of the atomic structure of the protein to a resolution better than 3.5 Å. Most preferably the crystal effectively diffracts x-rays for the determination of the atomic structure of the protein to a resolution of 3.6 Å, 3.2 Å or 2.8 Å.

In a first embodiment the crystal forms a complex with AlF₄⁻ and is characterised by the atomic coordinates as presented in Table 11. Thus, the three dimensional structure of the crystal according to this invention may in one embodiment be determined by the atomic coordinates as presented in Table 11.

In a second embodiment the crystal forms a complex with MgF₄²⁻ and is characterised by the atomic coordinates as presented in Table 12. Thus, the three dimensional structure of the crystal according to this invention may in one embodiment be determined by the atomic coordinates as presented in Table 12.

In a third embodiment the crystal forms a complex with BeF₃⁻ and is characterised by the atomic coordinates as presented in Table 13. Thus, the three dimensional structure of the crystal according to this invention may in one embodiment be determined by the atomic coordinates as presented in Table 13.

### Type IB P-type ATPases

The type IB P-type ATPases comprises the heavy metal transport ATPases that remove toxic ions such as Cu⁺, Ag⁺, Zn⁺, Cd⁺, Co⁺ or Pb⁺ from the cell. Most type IB P-type ATPases are bacterial, but close homologues have been found in yeast, plants and animals.

In one embodiment the present invention relates to a crystal comprising a type IB P-type ATPase, wherein the ATPase is a bacterial type IB P-type ATPase.

The bacteria may be any kind of bacterial species. In one preferred embodiment the bacteria is pathogenic bacteria. In another preferred embodiment the type IB P-type ATPase is a Legionella pneumophila type IB P-type ATPase.

In a particular embodiment the ATPase is the LPG1024 (SEQ ID NO:1) Legionella pneumophila ATPase or an ATPase having at least 85% sequence identity with SEQ ID NO:1 or a functional homologue thereof.

In a further embodiment, the crystal of the present invention may comprise an ATPase, wherein said ATPase is a functional homologue of the Legionella pneumophila ATPase having at least 75% sequence identity, for example at least 80% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with SEQ ID NO:1.

Proteins, which are functional homologues, are proteins with similar functions but not necessarily of shared evolutionary origin.

The invention further encompasses type IB P-type ATPase from different species such as yeast, plants or animals. Such ATPases from other species can be interpreted as functional homologues of the type IB P-type ATPase identified by SEQ ID NO 1. According to the inventions functional homologues of the ATPase identified by SEQ ID NO:1 also covers sequences obtained by modifications of a type IB P-type ATPase.

In another embodiment, the crystal of the present invention may comprise an ATPase, wherein said ATPase is a homologue of the Legionella pneumophila ATPase having at least 75% sequence identity, for example at least 80% sequence identity, for example at least 90% sequence identity, such as at least 91% sequence identity, for example at least 91% sequence identity, such as at least 92% sequence identity, for example at least 93% sequence identity, such as at least 94% sequence identity, for example at least 95% sequence identity, such as at least 96% sequence identity, for example at least 97% sequence identity, such as at least 98% sequence identity, for example 99% sequence identity with SEQ ID NO:1.

In a preferred embodiment the ATPase is a homologue of the Legionella pneumophila ATPase having at least 85% sequence identity with SEQ ID NO:1.

The sequence identity can be determined with the algorithms GAP, BESTFIT, or FASTA in the Wisconsin Genetics Software Package Release 7.0, using default gap weights.

The sequence identity is calculated by comparing two aligned sequences, determining the number of positions at which identical amino acid residues occurs in both sequences to yield the number of matched positions, dividing the number of matched positions by the total number of positions in the aligned sequence, and multiplying the result by 100 to yield the percentage of sequence identity.

The homology between amino acid sequences may be calculated using well known scoring matrices such as any one of BLOSUM 30, BLOSUM 40, BLOSUM 45, BLOSUM 50, BLOSUM 55, BLOSUM 60, BLOSUM 62, BLOSUM 65, BLOSUM 70, BLOSUM 75, BLOSUM 80, BLOSUM 85, and BLOSUM 90.

According to the invention the ATPase comprised by the crystal is not the necessarily a full-length protein. Truncated versions can readily be prepared by conventional methods of molecular biology. According to the invention it is preferred that the ATPase comprised by the crystal comprise more than 75%, more preferred 80%, and mostly preferred more than 90% of the full length protein sequence.

In one embodiment the type IB P-type ATPase comprised by the crystal is able to transport monovalent cations.

The type IB P-type ATPase comprised by the crystal may transport any of the metals selected from the group consisting of Zn⁺, Cd⁺, Co⁺ or Pb⁺.

In one preferred embodiment the type IB P-type ATPase of the invention is able to transport Ag⁺ and/or Au⁺.

In another preferred embodiment the type IB P-type ATPase is able to transport Cu⁺.

Thus, In one preferred embodiment the type IB P-type ATPase comprised by the crystal is a Cu⁺-ATPase. A Cu⁺-ATPase is a type IB P-type ATPase, which is able to transport Cu⁺-ions across the cell membrane.

In a particular preferred embodiment the type IB P-type ATPase is CopA. CopA is a bacterial or archaeal Cut-transporting ATPase that participates in the uptake of copper.

### Source

The protein material subjected to crystallization experiments according to the invention may be obtained from various sources, such as purified from animal, plant, fungal, bacterial or archaebacterial material.

Alternatively the ATPase may be produced by recombinant method known by a person skilled in the art. Recombination methods enable expression of proteins at a high level wherefore proteins for crystallization experiment is preferably obtain using recombinant methods. The protein may be expressed in a host different from the organism from where the gene is derived. Suitable hosts for heterogenic expression of proteins can be bacteria, fungi, yeast, plants and tissue culture cells.

According to the present invention the type IB P-type ATPase is preferably expressed in bacteria, more preferably in Escherichia coli.

### Purification of Protein

Independent of the source of the ATPase the protein must be purified before crystallization. The purification may be performed by conventional methods known in the art, which may differ dependent on the source of ATPase. The method of purification may depend on the use of one or more particular tags.

ATPases of the invention are transmembrane proteins and thus comprises domains, which are membrane integral as well as both intra- and extra cellular domains. Thus both hydrophilic and hydrophobic domains are present which complicates expression and purification of the protein. Detergents are usually required for solubilisation of membrane proteins, but such detergents often interfere with crystallization.

The applicant has success full established a procedure for expression, purification and crystallization of a type IB P-type ATPase.

The protein is expressed in Escherichia coli and the membrane fractions collected by a series of sequential centrifugation steps (se examples). The ATPase according to the invention is solubilised in a suitable detergent.

Preferred detergents include maltoside-based detergents such as dodecyl-maltoside (DDM), and 6-Cyclohexylhexyl-1-pentyl-β-D-maltoside (Cymal-6). In one preferred embodiment the type IB P-type ATPase is solubilised in octaethylene glycol monododecyl ether (C₁₂E₈).

An aspect of the invention relates to a method of purification of a type IB P-type ATPase comprising solubilising the ATPase using a polyoxyethylene lauryl ether and/or a maltoside-based detergent.

Thus, in one aspect the invention relates to method for purification of a type IB P-type ATPase comprising the following steps:
a. obtaining a composition comprising a type IB P-type ATPase,
b. solubilising said type IB P-type ATPase using a polyoxyethylene lauryl ether and/or a maltoside-based detergent,
c. purifying said type IB P-type ATPase.

In one preferred embodiment the polyoxyethylene lauryl ether is octaethylene glycol monododecyl ether (C₁₂E₈).

In another preferred embodiment the maltoside-based detergent is dodecyl-maltoside (DDM).

The method for purification may further comprise a step of treating said composition comprising a type IB P-type ATPase with at least 0.1 mg/ml of one or more lipids before growing the type IB P-type ATPase crystals. In one embodiment at least 0.5 mg/ml, such as 1 mg/ml, such as for example 1.5 mg/ml such as 2 mg/ml, such as for example 2.5 mg/ml or such as 3 mg/ml or even more of one or more lipids are used before growing the crystals. In another embodiment saturating amounts of one or more lipids are used.

In one embodiment the type IB P-type ATPase is treated with lipids extracted from E. coli cells or soy bean cells.

In one preferred embodiment the type IB P-type ATPase is treated with dioleoyl-phosphatidylcholine.

### Growing of the Crystal

Growing of a crystal comprising a type IB P-type ATPase may according to the invention be performed by for example vapour diffusions methods and/or hanging drops systems known by the person skilled in the art.

An aspect of the invention relates to a method of growing crystal comprising a type IB P-type ATPase. Such method includes the steps of obtaining an ATPase composition of sufficient quality for growing of a crystal and growing of ATPase crystals. As described herein, both steps can be modulated to optimise the outcome.

In an embodiment the invention relates to a method for growing a crystal comprising a type IB P-type ATPase comprising the steps of:
a. obtaining a composition comprising a type IB P-type ATPase,
b. growing type IB P-type ATPase crystals and thereby
b. obtaining a crystal comprising a type IB P-type ATPase.

In a preferred embodiment the invention relates to a method of growing a crystal comprising a type IB P-type ATPase, comprising the steps of:
a. obtaining a composition comprising a type IB P-type ATPase,
b. growing type IB P-type ATPase crystals in a crystallization environment including PEG and
c. obtaining crystals comprising a type IB P-type ATPase

In one preferred embodiment the polyoxyethylene lauryl ether is octaethylene glycol monododecyl ether (C₁₂E₈).

In another preferred embodiment the maltoside-based detergent is dodecyl-maltoside (DDM).

It is preferred that the incubation with dioleoyl-phosphatidylcholine is performed in the presence if C₁₂E₈, preferably 0,5 mg C₁₂E₈ per mg protein (ATPase).

When growing the crystals in the crystallization environments as described in the method above, a precipitant buffer is used. The precipitant buffer comprises a precipitant such as for example PEG.

PEG, which is used as a precipitant, may be selected from the group of PEGs comprising: PEG 100, PEG 200, PEG 400, PEG 600, PEG 800, PEG 1000, PEG 2000, PEG 3000, PEG 4000, PEG 5000, PEG 7000, PEG 8000 PEG, 8000 PEG 10000, PEG 15000 and PEG 20000. Likewise poly(ethyleneglycol) methyl ethers (PMEs) and/or monomethyl ethers (MMEs) may be used as precipitants. For example PEG 800 MME, PEG 1000 MME, PEG 3000 MME, PEG 4000 MME, PEG 5000 MME, PEG 7000 MME, PEG 8000 PEG MME may be used.

In one preferred embodiment PEG 6000 is used. In another preferred embodiment polyethylene glycol 2000 monomethyl ether (PEG 2000 MME) is used.

Step b in the method described above may further comprise growing type IB P-type ATPase crystals by vapour diffusion from hanging drops with a reservoir buffer comprising a precipitant such as for example PEG, PME or MME as described above. The recevoir buffer may also be referred to as the recevoir solution, the precepitant buffer or the precipitant solution.

In one embodiment the concentration of PEG in the recevoir buffer is 2-12% (w/v), such as 4-9% (w/v) or 5-7% (w/v). In another embodiment the concentration of PEG 2000 MME in the recevoir buffer is 2-20% (w/v), such as 6-16% (w/v) or 10-15% (w/v).

In a first preferred embodiment the reservoir buffer comprises 5-7% (w/v) PEG. The reservoir buffer may for example comprise 5-7% (w/v) PEG, 80-200 mM NaCl, 3% (v/v) t-BuOH and 5 mM BME. It is preferred that this reservoir buffer is used for growing crystals complexed with AlF₄⁻.

In a second preferred embodiment the reservoir buffer comprises 14% (w/v) PEG 2000 MME. The reservoir buffer may for example comprise 14% (w/v) PEG 2000 MME, 200 mM KCl, 3% (v/v) t-BuOH and 5 mM BME. It is preferred that this reservoir buffer is used for growing crystals complexed with BeF₃⁻.

In a third preferred embodiment the reservoir buffer comprises 11% (w/v) PEG 2000 MME. The reservoir buffer may for example comprise 11% (w/v) PEG 2000 MME, 200 mM KCl and 5 mM BME. It is preferred that this reservoir buffer is used for growing crystals complexed with MgF₄^{2-.}

In a preferred embodiment the hanging drop experiment is sealed by vacuum grease or other sealant with low permeability (as compared to immersion oil). Most preferably the hanging drop experiment is set up and incubated at 20° C. In the optimal procedure for the hanging drop experiment is initiated by mixing 1 μl reservoir solution and 1 μl protein solution and placing the supernatant in the hanging drop chamber.

Initiation of crystal formation, also known as nucleation can be performed by lowering the solubility of the ATPase. According to the invention PEG is included in the crystallization environment.

In order to initiate crystallization of proteins various precipitating agents can be used. The precipitating agent is preferably included in the crystallization environment. The precipitating agent may be comprised by the buffer of the reservoir, when the crystals are grown by the vapour diffusion method.

The crystal structure of Cu⁺-ATPase complexed with AlF₄⁻ from Legionella pneumophila was obtained as described in the Example 1 here below. The data are summarized in Table 1.

Those of skill in the art will understand that a set of structure coordinates for a protein or protein complex or a portion thereof, is a relative set of points that define a shape in three dimensions. Thus, it is possible that an entirely different set of coordinates could define a similar or identical shape. The variations in coordinates may be generated by mathematical manipulations of the structure coordinates. For example, the structure coordinates set forth in Table 1 could be manipulated by crystallographic permutations of the structure coordinates, fractionalization or matrix operations to sets of the structure coordinates or any combination of the above.

### Coordinates Stored on Machine Readable Storage Medium

In a further aspect the invention provide a computer-readable data storage medium comprising a data storage material encoded with the structure coordinates, or at least a portion of the structure coordinates set forth in Table 1. Examples of such computer readable data storage media are well known to those skilled in the art and include, for example CD-ROM and diskette (“floppy disks”). Thus, in accordance with the present invention, the structure coordinates of an ATPase, and portions thereof can be stored in a machine-readable storage medium. Such data may be used for a variety of purposes, such as drug discovery and X-ray crystallographic analysis of protein crystal.

The storage medium may further be local to a computer or the storage medium may be located in a net-worked storage medium including the internet, to which remote accessibility is possible.

### Use of Crystal

Provided that crystals of sufficient quality have been obtained, the crystals may according to the invention be used for X-ray diffraction experiments.

An aspect of the invention relates to the use of a crystal comprising a type III P-type ATPase for determination of the three dimensional structure of said ATPase.

Before data collection crystals may be treated by standard methods known in the art, which include preparation of samples for heavy atom derivatization by dusting a dry powder of Ta₆Br₁₂ or solutions containing iridium, platinum or mercury.

The crystals are thereafter mounted in nylon or litho loops and flashed cooled in liquid nitrogen.

Data collection and data processing can be performed by any suitable systems known by the person skilled in the art. Data may be collected using the Swiss Light Source X06SA beamline on a Mar225 CCD detector. Processing may be performed using XDS³¹. Data processing is further described in the examples.

### Identification of Inhibitors

According to the invention various strategies can be followed to identify and generate inhibitors of a type IB P-type ATPases based on the structural information described herein.

An aspect of the invention relates to the use of a crystal according to this invention for identifying inhibitors of a type IB P-type ATPase.

One aspect of the present invention relates to use of atomic coordinates as presented in Table 1 or atomic coordinates selected from a three-dimensional structure that deviates from the three-dimensional structure as presented in Table 1 by a root mean square deviation over protein backbone atoms of not more than 3 Å, in a method for identifying a inhibitor of a type IB P-type ATPase.

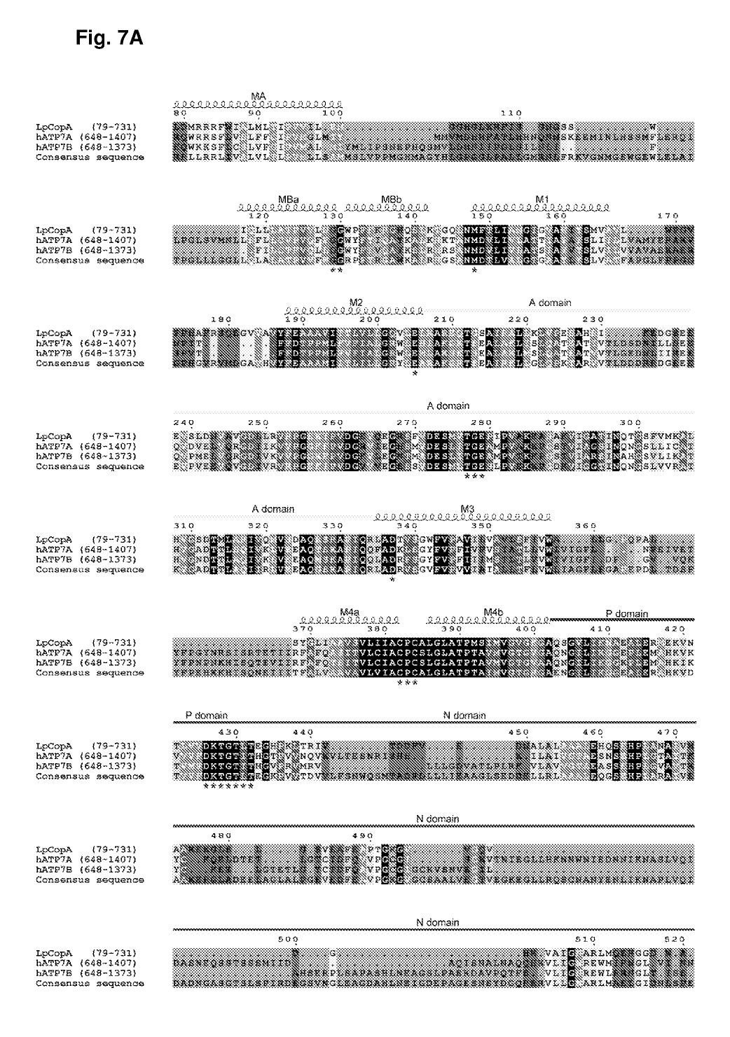

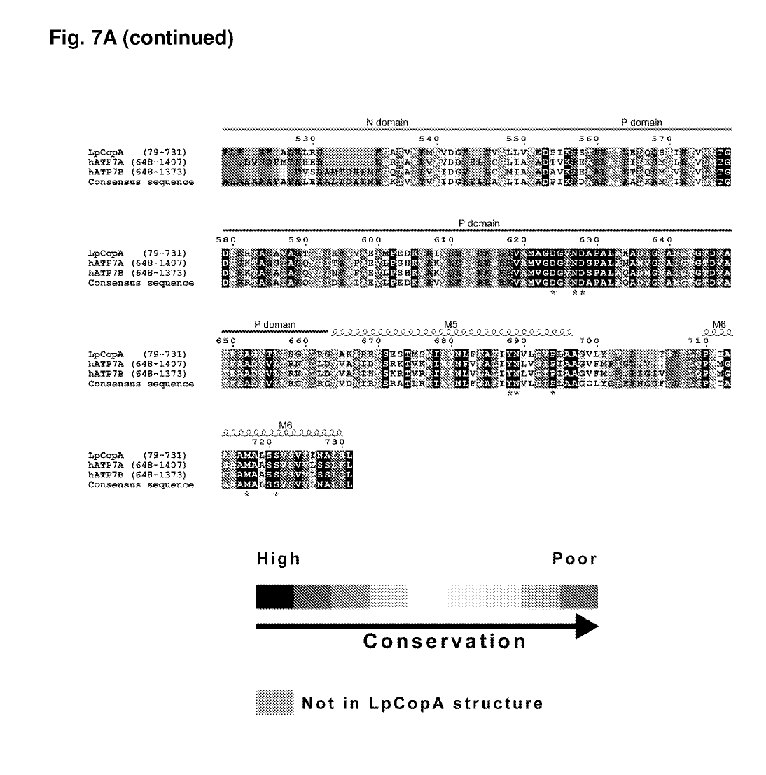

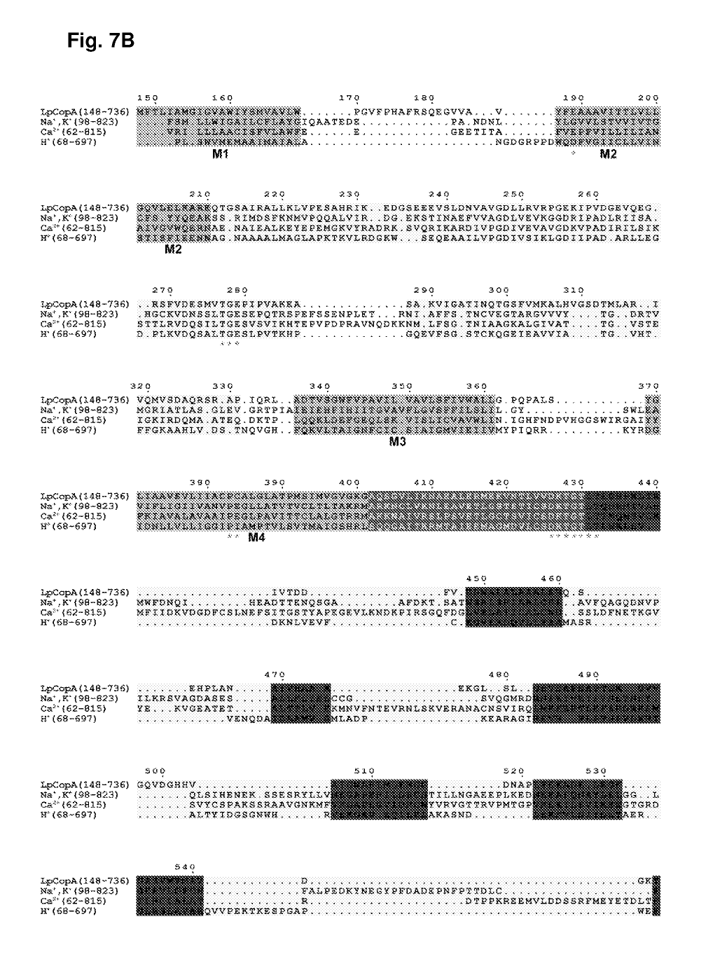

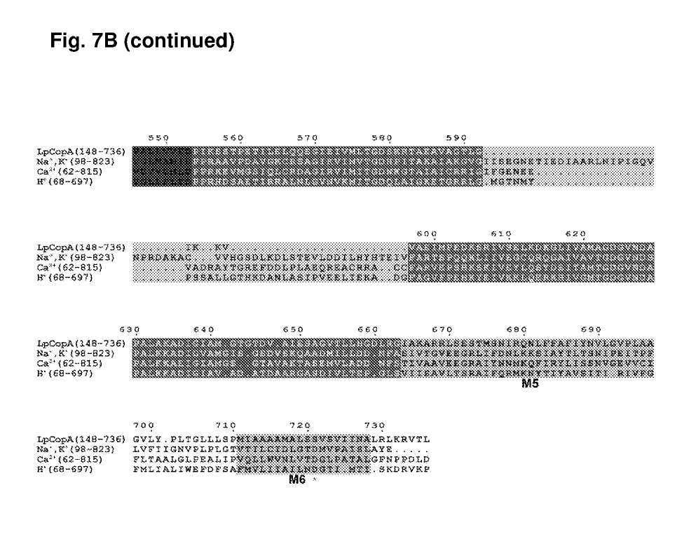

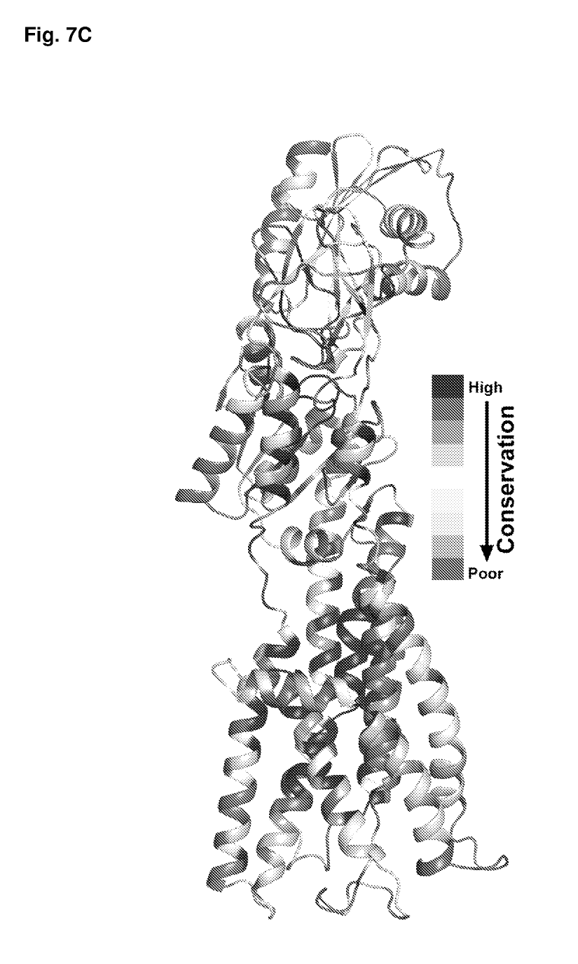

In one embodiment the use of atomic coordinates as presented in Table 1 or atomic coordinates selected from a three-dimensional structure deviates from the three-dimensional structure as presented in Table 1 by a root mean square deviation over protein backbone atoms of not more than 3 Å for residues in conserved segments according to sequence alignment as presented in FIG. 7.

To identify inhibitors of a type IB P-type ATPase the crystal may be treated or contacted with various compounds.

Thus, in one embodiment the use of the crystal of this invention, for identifying inhibitors of a type IB P-type ATPase, comprises a step of contacting said crystal with one or more compounds. Such compounds could for example be inhibitors or potential inhibitors of the ATPase. Examples of compounds are vanadate, ATP/ADP and phosphate analogues such as AMP-PCP, BeF₃, AlF₄, and MgF₄.

For example inhibitors or potential inhibitors can be co-crystallised with a type IB P-type ATPase to see how the inhibitor binds to the ATPase. In that way it is possible to further optimize the ability of the inhibitor or the potential inhibitor to inhibit the activity of the ATPase.

Inhibitors that can bind to for example the entry site, the metal binding site or the exit site can be identified through virtual screening of chemical databases. Virtual screening are performed with different database docking programs (for instance Dock, FlexX, Gold, Flo, Fred, Glide, LigFit, MOE or MVP, but not limited to these) and used with different scoring functions (e.g. Warren et. al., 2005; Jain, 2006; Seifert et al., 2007). The scoring functions may include, but are not limited to force-field scoring functions (affinities estimated by summing Van der Waals and electrostatic interactions of all atoms in the complex between the type IB P-type ATPase and the ligand), empirical scoring functions (counting the number of various interactions, for instance number of hydrogen bonds, hydrophobic-hydrophobic contacts and hydrophilic-hydrophobic contacts, between the type IB P-type ATPase and the ligand), and knowledge based scoring functions (with basis on statistical findings of intermolecular contacts involving certain types of atoms or functional groups). Scoring functions involving terms from any of the two of the mentioned scoring functions may also be combined into a single function used in database virtual screening of chemical libraries.

Knowledge about the three dimensional crystal structure described herein provides basis for the identification of inhibitors of type IB P-type ATPases. It is preferred that the structure used is based on the atomic coordinates presented in Table 1, but a structure that deviates from the three-dimensional structures as presented in Table 1 by a root mean square deviation over protein backbone atoms of not more than 3 Å may like wise be used. It is preferred that the deviate is less than 2 Å, more preferably less than 1 Å.

Such methods are preferable performed using computers, whereby the atomic coordinates are introduced into the computer, allowing generation of a model on the computer screen which allows visual selection of binding molecules.

### Methods of Selecting or Identifying Inhibitors

Preferably, inhibitors are selected by their potential of binding to the entry site, the exit site or the metal binding site of the type IB P-type ATPases. When selecting an inhibitor by computer modelling, the 3D structure of the ATPase is loaded from a data storage device into a computer memory and may be displayed (generated) on a computer screen using a suitable computer program. Preferably, only a subset of interest of the coordinates of the whole structure of the ATPase is loaded in the computer memory or displayed on the computer screen. This subset may be called a criteria data set; this subset of atoms may be used for designing an inhibitor.

An aspect of the present invention relates to a method of identifying an inhibitor of a type IB P-type ATPase by determining binding interactions between the inhibitor and a set of binding interaction sites in said type IB P-type ATPase comprising the steps of:
a. generating the spatial structure of the type IB P-type ATPase on a computer screen using atomic coordinates as presented in Table 1 or atomic coordinates selected from a three-dimensional structure that deviates from the three-dimensional structure as presented in Table 1 by a root mean square deviation over protein backbone atoms of not more than 3 Å,
b. generating the spatial structure of inhibitors on the computer screen, and
c. selecting inhibitors that can bind to at least one amino acid residues of the set of binding interaction sites with out steric interference.

In an alternative aspect the inhibitors are identified using a computer, wherein the computer comprise programs and processor capable of utilizing the three dimensional structure information for selecting inhibitors bases on a criteria data set which defines target regions of the ATPase. Data base of inhibitors, such as data bases of low molecular weight organic chemical structures can be stored in the computer, e.g. in a storage system and used by the processor of the computer to identify inhibitors which, in a region are structurally complementary to the criteria data set and being free of steric interference with the ATPase. Inhibitors being, in a region, complementary to the criteria data set, can be interpreted as inhibitors capable of accommodating a three-dimensional cavity defined by the criteria data set with out interfering with the structure of the target. Complementary indicates that the ATPase and the Inhibitors interact with each other in an energy favourable way minimizing the availability of polar and charged residues (se below). The storage medium may be local to the computer as described above, or the storage medium may be remote such as a net-worked storage medium including the internet.

The low molecular weight organic chemical structures may include, but are not limited to, structures such as lipids, nucleic acids, peptides, proteins, antibodies and saccharides.

A further method according to the invention relates to a computer-assisted method for identifying inhibitors of a type IB P-type ATPase using a programmed computer processor, a data storage system, a data input devise and a data output devise comprising the following steps:

a. inputing into the programmed computer through said input device data comprising: a subset of the atoms of a type IB P-type ATPase, thereby generating a criteria data set, wherein the atomic coordinates are selected from the three-dimensional structure as presented in Table 1 or atomic coordinates selected from a three-dimensional structure that deviates from the three-dimensional structures as presented in Table 1 by a root mean square deviation over protein backbone atoms of not more than 3 Å,
b. comparing, using said processor, the criteria data set to a computer data base of low molecular weight organic chemical structures stored in the data storage system; and
c. selecting from said data base, using computer methods, a chemical structure having a portion that is structurally complementary to the criteria data set and being free of steric interference with the type IB P-type ATPase.

The invention further relates to a computer-assisted method for identifying inhibitors of a type IB P-type ATPase using a programmed computer processor, a data storage system, a data input devise and a data output devise comprising the following steps:
a. inputting into the programmed computer through said input device data comprising: a subset of the atoms of a type IB P-type ATPase, thereby generating a criteria data set; wherein the atomic coordinates are selected from the three-dimensional structure as presented in Table 1 or atomic coordinates selected from a three-dimensional structure that deviates from the threethree-dimensional structures as presented in Table 1 by a root mean square deviation over protein backbone atoms of not more than 3 Å,
b. comparing, using said processor, the criteria data set to a computer data base of low molecular weight organic chemical structures stored in the data storage system; and
c. constructing using computer methods a model for a chemical structure having a portion that is structurally complementary to the criteria data set and being free of steric interference with the type IB P-type ATPase.

A further method according to the invention relates to a computer-assisted method for identifying inhibitors of a type IB P-type ATPase using a programmed computer processor, a data storage system, a data input devise and a data output devise comprising the following steps:
a. inputing into the programmed computer through said input device data comprising: a subset of the atoms of a type IB P-type ATPase, thereby generating a criteria data set, wherein the atomic coordinates are selected from the three-dimensional structure as presented in Table 1 or atomic coordinates selected from a three-dimensional structure that deviates from the three-dimensional structures as presented in Table 1 by a root mean square deviation over protein backbone atoms of not more than 3 Å,
b. comparing, using said processor, the criteria data set to a computer data base of low molecular weight organic chemical structures stored in the data storage system; and
c. selecting from said data base, using computer methods, a chemical structure having a portion that is structurally complementary to the criteria data set and being free of steric interference with the type IB P-type ATPase and/or
d. constructing using computer methods a model for a chemical structure having a portion that is structurally complementary to the criteria data set and being free of steric interference with the type IB P-type ATPase.

In one embodiment the criteria data set or the binding interaction sites may comprise one or more amino acid residues selected from the group comprising: Glu189, Met711, Met100 and Glu99 of SEQ ID NO:1.

Glu189, Met711, Met100 and/or Glu99 may be involved in the final binding or coordination of a monovalent cation such as for example Cu⁺ and may be part of an exit site. The exit site comprising Glu189, Met711, Met100 and/or Glu99 is localised on the extracellular site of the cell membrane.

The entry site as used herein is the site for entry of the metal, which is transported from the intracellular site to the extracellular site (the cytoplasm) of the cell membrane.

The extra cellular site of the crystal comprising the type IB P-type ATPase is an important binding site for an inhibitor of said ATPase since binding of an inhibitor to the extra cellular site of the type IB P-type ATPase will prevent the development of resistance to the inhibitor. Drug resistance in bacteria is often mediated by efflux-pumps, which are able to remove the drug from the cell by transporting the drug from the intracellular site to the extracellular site of the cell membrane. Thus, drug resistance mediated by efflux pump is not possible when the drug or the inhibitor functions by binding to the extracellular part of a transmembrane protein.

Thus, in one embodiment the criteria data set may comprise the extracellular site of the crystal as described herein.

In another embodiment the criteria data set may comprise one or more amino acid residues selected from the group comprising: Gly129 and Gly130 of SEQ ID NO:1. Gly129 and Gly130 are localised at the intracellular site of the cell membrane.

In further embodiment the criteria data set or the binding interaction sites may comprise one or more amino acid residues selected from the group comprising: Cys382, Pro383, Cys384, Tyr688, Asn689, Met717, Ser721. Cys382, Pro383, Cys384, Tyr688, Asn689, Met717, Ser721 are localised in the transmembrane domain of the ATPase and may be involved in the binding or coordination as well as in the transport of a monovalent cation such as for example Cu⁺ and may as such also serve as potential drug targets.

In a further embodiment the criteria data set or the binding interaction sites may comprise one or more amino acid residues selected from the group comprising: Thr277, Gly278, Glu279, Asp426, Lys427, Thr428, Gly429, Thr430 and Thr432. Thr277, Gly278, Glu279, Asp426, Lys427, Thr428, Gly429, Thr430 and Thr432 are localised at the intracellular site of the cell membrane. These amino acids are critical for enzyme phosphorylation/dephosphorylation and may as such also serve as potential drug targets.

In a further embodiment the criteria data set or the binding interaction sites set may comprise one or more amino acid residues selected from the group comprising: Met148, Asp337 and Glu205. Met148, Asp337 and/or Glu205 may be involved in the initial binding or coordination of a monovalent cation such as for example Cu⁺ and may be part of a pre-coordination site or the entry site. The pre-coordination site comprising Met148, Asp337 and/or Glu205 is localised on the intracellular site of the cell membrane and may as such also serve as potential drug target.

The groups of amino acids which are mentioned herein as being comprised by the criteria data set and the binding interaction may also comprise corresponding amino acids from other type IB P-type ATPases. Such corresponding amino acids can be identified by sequence alignments of amino acid sequences from different type IB P-type ATPases with SEQ ID NO:1.

In the methods described herein the one or more amino acid residues comprised by the data criteria set may be at least one, or at least two, preferably at least 3, more preferably at least 4 or 5 or mostly preferred at least at least 6, 7 or 8 AA selected from the identified groups.

An inhibitor may then be designed de novo in conjunction with computer modelling. Models of chemical structures or molecule fragments may be generated on a computer screen using information derived from known low-molecular weight organic chemical structures stored in a computer data base or are built using the general knowledge of an organic chemist regarding bonding types, conformations etc. Suitable computer programs may aid in this process in order to build chemical structures of realistic geometries. Chemical structures or molecule fragments may be selected and/or used to construct an inhibitor such that favourable interactions to said subset or criteria data set become possible. The more favourable interactions become possible, the stronger the inhibitor will bind to the ATPase. Preferably, favourable interactions to at least one amino acid residues should become possible. Such favourable interactions may occur with any atom of the amino acid residue e.g. atoms of the peptide back-bone or/and atoms of the side chains.

Favourable interactions are any non-covalent attractive forces which may exist between chemical structures such as hydrophobic or van-der-Waals interactions and polar interactions such as hydrogen bonding, salt-bridges etc. Unfavourable interactions such as hydrophobic-hydrophilic interactions should be avoided but may be accepted if they are weaker than the sum of the attractive forces. Steric interference such as clashes or overlaps of portions of the inhibitor being selected or constructed with protein moieties will prevent binding unless resolvable by conformational changes. The binding strength of an inhibitor thus created may be assessed by comparing favourable and unfavourable interactions on the computer screen or by using computational methods implemented in commercial computer programs.

Conformational freedom of the inhibitor and amino acid side chains of the ATPase should be taken into account. Accessible conformations of an inhibitor may be determined using known rules of molecular geometry, notably torsion angles, or computationally using computer programs having implemented procedures of molecular mechanics and/or dynamics or quantum mechanics or combinations thereof.

An inhibitor is at least partially complementary to at least a portion of the active site of the ATPase in terms of shape and in terms of hydrophilic or hydrophobic properties. Databases of chemical structures (e. g. cambridge structural database or from Chemical Abstracts Service; for a review see: Rusinko (1993) Chem. Des. Auto. News 8,44-47) may be used to varying extents. In a totally automatic embodiment, all structures in a data base may be compared to the active site or to the binding pockets of the ATPase for complementarity and lack of steric interference computationally using the processor of the computer and a suitable computer program. In this case, computer modelling which comprises manual user interaction at a computer screen may not be necessary. Alternatively, molecular fragments may be selected from a data base and assembled or constructed on a computer screen e. g. manually. Also, the ratio of automation to manual interaction by a person skilled in the art in the process of selecting may vary a lot. As computer programs for drug design and docking of molecules to each other become better, the need for manual interaction decreases.

A preferred approach of selecting or identifying inhibitors of type IB P-type ATPases makes use of the crystal according to this invention. Analogously to the principles of drug design and computer modelling outlined above, chemical structures or fragments thereof may be selected or constructed based on non-covalent interactions between the inhibitor and the type IB P-type ATPase.

Inhibitors may be selected or designed such that they interfere with binding of and organic compound bound by the ATPase, such as ATP or an ATP analogues such as AAMPPCP or alternatively any cations associated with the ATPase. Such inhibitors may prevent binding of ATP or ATP analogues or cations the ATPase.

Programs usable for computer modelling include Quanta (Molecular Simulations, Inc.) and Sibyl (Tripos Associates). Other useful programs are Autodock (Scripps Research Institute, La Jolla, described in Goodsell and Olsen (1990) Proteins: Structure, Function and Genetics, 8, 195-201), Dock (University of California, San Francisco, described in: Kuntz et al. (1982) J. Mol. Biol. 161,269-288.

In one aspect the present invention relates to a method for identifying an inhibitor capable of inhibiting the Cu⁺ translocating activity of a type IB P-type ATPase, said method comprising the following steps:
a. identifying an inhibitor using atomic coordinates in conjunction with computer modelling, wherein said atomic coordinates are the atomic coordinates presented in Table 1 or wherein the atomic coordinates are selected from a three-dimensional structure that deviates from the three-dimensional structures presented in Table 1 by a root mean square deviation over protein backbone atoms of not more than 3, by docking inhibitors into a set of binding interaction sites in a Cu⁺ transfer pathway generated by computer modelling and selecting an inhibitor capable of binding to at least one amino acid in said Cu⁺ transport pathway,
b. contacting said inhibitor with said type IB P-type ATPase and
c. detecting inhibition of Cu+ translocating activity of said type IB P-type ATPase by said inhibitor.

In a preferred embodiment docking of inhibitor molecules is performed by employing the type IB P-type ATPase crystal defined by atomic coordinates presented in Table 1 and such that said inhibitor is capable of binding to at least three amino acid in the Cu⁺ transport pathway.

Another aspect the present invention relates to a method for identifying an inhibitor capable of inhibiting the activity of a type IB P-type ATPase, said method comprising the following steps:
a. introducing into a computer information derived from atomic coordinates presented in Table 1 or atomic coordinates selected from a three-dimensional structure that deviates from the three-dimensional structures as presented in Table 1 by a root mean square deviation over protein backbone atoms of not more than 3 Å,
b. generating a three-dimensional structure using said atomic coordinates,
c. superimposing a model of an inhibitor on said three-dimenssional structure;
d. assessing the possibility of binding and the absence of steric interference of the inhibitor with the type IB P-type ATPase;
e. incorporation said inhibitor in an activity assay of said type IB P-type ATPase and
f. determining whether said inhibitor inhibits the activity of said type 1B P-type ATPase.

In one embodiment information is derived from the atomic coordinates of at least one of the amino acid residues selected from the group comprising: Met148, Asp337 and Glu205. Met148, Asp337 and Glu205 may be involved in the initial binding or coordination of a monovalent cation such as for example Cu⁺ and may be part of a pre-coordination site.

In one preferred embodiment information is derived from the atomic coordinates of at least one of the amino acid residues of the extracellular site of the crystal as described herein. Thus, in one embodiment the inhibitor binds to the extracellular site of the crystal of the invention.

In another preferred embodiment information is derived from the atomic coordinates of at least one of the amino acid residues selected from the group comprising: Gly129 and Gly130 of SEQ ID NO:1. Gly129 and Gly130 are localised at the intracellular site of the cell membrane.

In another embodiment information is derived from the atomic coordinates of at least one of the amino acid residues selected from the group comprising: Glu189, Met711, Met100 and Glu99 of SEQ ID NO:1

In another embodiment information is derived from the atomic coordinates of at least one of the amino acid residues selected from the group comprising: Cys382, Pro383, Cys384, Tyr688, Asn689, Met717 and Ser721 of SEQ ID NO:1.

In a further embodiment information is derived from the atomic coordinates of at least one of the amino acid residues selected from the group comprising: Thr277, Gly278, Glu279, Asp426, Lys427, Thr428, Gly429, Thr430 and Thr432 of SEQ ID NO:1.

The groups of amino acids which are mentioned herein may also comprise corresponding amino acids from other type IB P-type ATPases. Such corresponding amino acids can be identified by sequence alignments of amino acid sequences from different type IB P-type ATPases with SEQ ID NO:1.

As described above the data criteria sets described herein may be used for defining the group of residues for which the atomic coordinates are included.

More preferably information derived from at least 2, such as at least 3 amino acid residues in the Cu⁺ transport pathway are used in the methods. In an even further preferred embodiment information regarding the special localisation for more than 3, such as more than 4, or more than 5 amino acids residues are used in the methods.

In one embodiment the data criteria set or binding interaction set comprise at least 3 amino acid residues selected from the identified groups.

It is preferred that the resolution of the atomic coordinates are determined to a resolution of at least 4 Å, more preferably at least 3,5 Å or at least 3 Å or better.

In one preferred embodiment the atomic coordinates are determined to a resolution, of at least 3,5 Å.

Inhibitors selected according to the invention preferably interacts with at least 1, more preferably at least 2, or further preferred as at least 3 amino acids in the Cu+ transport pathway or mostly preferred at least 4 amino acids in the Cu+ transport pathway.

### Type IB P-Type ATPase Specific Inhibitors

In order to identify inhibitors specific for the type IB P-type ATPase, that is an inhibitor which do not inhibit different types of ATPases such as for example the H⁺, Na⁺, ATPase, or the Ca²⁺ ATPase, structural information regarding these ATPases may be used in the methods described herein. The specificity may following be tested in vivo or in vitro assays as described in relation to verification of inhibitors.

It is possible to use sequence information, such as sequence identities and sequence differences between different type IB P-type ATPase to develop inhibitors that are specific for different families, such as bacteria, fungi, yeast or plant, or even different species within a family.

The screening of different libraries can also be performed using different ATPase for selection of specific inhibitors.

A further aspect of the invention relates to a method for identifying a selective peptide inhibitor of a type IB P-type ATPase comprising the following steps:
a. identification of an inhibitors of a type IB P-type ATPase according to any of the claims,
b. contacting the peptide inhibitors with said type IB P-type ATPase,
c. contacting the peptide inhibitors with a different type IB P-type ATPase,
d. detecting inhibition of type IB P-type ATPase activity of said type IB P-type ATPase by the inhibitors and
e. detecting activity of said different type IB P-type ATPase in the presence of said inhibitors.

A selective inhibitor is an inhibitor, which inhibits the activity of only one type of type IB P-type ATPase. In one preferred embodiment the selective inhibitor only inhibits the activity of Cu⁺ ATPases. Thus, the selective inhibitor may specifically inhibit the activity of Cu⁺ ATPases or the selective inhibitor may specifically recognise Cu⁺ ATPases.

The selective peptide inhibitor may be identified according to the methods described herein above.

In one embodiment the inhibitor is capable′ of inhibiting growth of bacteria having type IB P-type ATPases in their cell membrane. It is preferred that the bacteria are pathogenic bacteria.

### Screening of Libraries

Inhibitors of the type IB P-type ATPase may be identified by screening of libraries, or combinations of computer implemented methods and screening procedures. This is performed in vitro using membrane localized as well as purified fungal and plant plasma membrane Cu⁺-ATPases.

An aspect of the invention relates to a method of identifying inhibitors of a type IB P-type ATPase including a step of screening of different types of libraries known in the art. Different libraries may be screened according to the invention.

In one embodiment a library of small organic molecules are screened.

In a further preferred embodiment a library of peptide inhibitors are screened.

Compounds from the libraries are evaluated with respect to their effect upon plasma membrane Cu⁺-ATPase activity. The method may be combined with the in silicon methods described above. Such library screening method may be used to improve the identified inhibitor, e.g. to find inhibitors with a higher specificity or specificity to particular ATPases, such as ATPase from specific species for which an inhibitor is desirable (se further below in relation to verification of inhibitors).

### Methods for Testing the Inhibitory Activity of Identified Inhibitors

It is preferred that inhibitors identified according to the methods described herein and above are tested for their ability to inhibit the activity of a type IB P-type ATPase

In one preferred embodiment the inhibitory activity of identified inhibitors is identified by testing the ATPase activity of a type IB P-type ATPase in the presence of inhibitor according to the method as described in Example 3.

The inhibitory activity of identified inhibitors may be verified by state of the art techniques (se below). Thus, in vitro verification may include one or more of the following, but is not limited to tests of test of inhibition of ATP (or pNPP) hydrolytic activity, test of inhibition of metal ion transport, such as Cu⁺ transport, test of inhibitor binding affinity, test of inhibition of phosphorylation from ATP and/or test of inhibition of conformational transitions.

The potency of an inhibitor directed against a type IB P-type ATPase can for instance be tested in an ATPase (or any hydrolysable compound) assay. In an ATPase assay, the adenosine triphosphate (ATP) hydrolytic activity of the Cu⁺-ATPase is determined. ATP hydrolysis and metal ion pumping by type IB P-type ATPases are under normal circumstances strictly coupled and, therefore, ATP hydrolytic activity is a measure of the pumping activity. The ability of type IB P-type ATPase preparations to hydrolyse ATP can be tested in situ in isolated membranes, or in a detergent-solubilized purified form of the ATPase.

ATPase activity can be assayed by a variety of methods known by a skilled person in the art. Typically, one may quantify the time dependent release of breakdown products resulting from ATP hydrolysis, namely inorganic phosphate (Pᵢ) and adenosine diphosphate (ADP).

Time dependent release of Pᵢ from ATP is a convenient assay for ATPase activity. One assay known in the state of the art, benefits from the fact that P, forms complexes with molybdate that are blue when reduced. Alternatively, ATPase activity can be determined by following the time-dependent release of ADP. One assay, known in the state of art, enzymatically couples ADP formation to NADH oxidation.

The potency of type IB P-type ATPase inhibitors can also be tested by assaying their effect on metal ion pumping. Pump assays require that the type IB P-type ATPase is embedded in the membrane of a lipid vesicle, either derived from the plasma membrane of natural host cells or a heterologous host expressing the type IB P-type ATPase gene, or, alternatively, detergent-solubilized purified type IB P-type ATPase is reconstituted into an artificial lipid vesicle (Perlin et al., 1984). In all cases, the ATP binding site has to face the extravesicular medium so that ATP supplied to the medium can initiate ATP dependent metal ion accumulation into the lipid vesicles (also called liposomes).

Common to all P-type ATPases is the formation of a phosphorylated intermediate during the reaction cycle. The effect of ligands of type IB P-type ATPases can be assayed by their effect upon the formation, the steady-state amount or the decay of the phosphorylated intermediate.

The decay of the phosphorylated intermediate can be followed by stopping phosphorylation from [³²P]ATP with for instance cold ATP at different time points and the radioactivity (linear related to the amount of phosphorylated intermediate) measured as described. Testing the potential of ligands to interfere with conformational transitions of the type IB P-type ATPase can be tested in this phosphorylation assay. When ligands blocks conformational transitions of the ATPase, particular conformational transitions will accumulate. Thus, if an identified ligand for instance blocks enzymatic transitions away from the phosphorylated state, but not phosphorylation, a high amount of the phosphorylated form of the ATPase will accumulate.

Inhibitor binding can also be assayed directly by using radiolabelled ligands. Radiolabelled ligand binding studies is widely used to characterize the biochemical and pharmacological properties of ligand-protein complexes. In this way identified type IB P-type ATPase inhibitors can be tested by isotopically labelling the ligand, and its interaction with the type IB P-type ATPase can be directly monitored. Such a technology is fairly straightforward for a skilled person, and can provide accurate measurements of binding constants between the ligand in question and the type IB P-type ATPase.

In vivo verification may be shown by administration of inhibitors to diverse fungi and plants. In addition, in vivo effects of identified inhibitors may be shown in a yeast system where cell survival is tailored to be dependent upon the functionality of heterologous plasma membrane Cu⁺-ATPases. Recombinant methods may be employed for expression and testing the inhibitory activity on Cu⁺ pumps from different families and/or different species or even different genes from the same species.

The inhibitors can be synthesized according to the methods of organic chemistry. Preferably, compounds from a database have been selected without remodelling, and their synthesis may already be known.

In any event, the synthetic effort needed to find an inhibitor is greatly reduced by the achievements of this invention due to the pre-selection of promising inhibitors by the above methods. Binding of an inhibitor may be determined after contacting the inhibitor with the ATPase. This may be done crystallographically by soaking a crystal of the ATPase with the inhibitor or by co-crystallization and determining the crystal structure of the complex. Preferably, binding may be measured in solution according to methods known in the art. More preferably, inhibition of the catalytic activity of the ATPase by the inhibitor is determined e.g. using the assays described in the examples section.

In one preferred embodiment the identified inhibitors are able to inhibit the activity of a Cu⁺ ATPase. In another preferred embodiment the identified inhibitors are able to inhibit the Cu⁺ transport of a Cu⁺ ATPase.

### Use of the Inhibitor

One aspect of the present invention relates to use of an inhibitor as described herein for treatment of an individual infected with pathogenic bacteria having type IB P-type ATPases in their cell membrane.

Pathogenic bacteria are bacteria that can cause a bacterial infection or bacterial disease. The Cu⁺ ATPase is found in the cell membrane of all pathogenic bacteria. In one embodiment the pathogenic bacteria are conditionally pathogenic bacteria, which are only pathogenic under certain conditions. Such conditions may include a wound that allow for entry into the blood or a decrease in the immune function. Conditionally pathogenic bacteria include

In another embodiment the pathogenic bacteria are human pathogenic bacteria. Human pathogenic bacteria are bacteria that can cause a bacterial infection or bacterial disease in humans. Human pathogenic bacteria include Bordetella pertussis, Borrelia burgdorferi, Brucella abortus, Brucella canis, Brucella melitensis, Brucella suis, Campylobacter jejuni, Chlamydia pneumoniae, Chlamydia psittaci, Chlamydia trachomatis, Clostridium botulinum, Clostridium botulinum, Clostridium difficile, Clostridium perfringens, Clostridium tetani, Corynebacterium diphtheriae, Enterococcus faecalis, Enterococcus faecium, Escherichia coli, Enterotoxigenic Escherichia coli, Enteropathogenic E. coli, E. coli (O157:H7), Francisella tularensis, Haemophilus influenzae, Helicobacter pylori, Legionella pneumophila, Leptospira interrogans, Listeria monocytogenes, Mycobacterium leprae, Mycobacterium tuberculosis, Mycoplasma pneumoniae, Neisseria gonorrhoeae, Neisseria meningitides, Pseudomonas aeruginosa, Rickettsia rickettsii, Salmonella typhi, Salmonella typhimurium, Shigella sonnei, Staphylococcus aureusa, Staphylococcus epidermidis, Staphylococcus saprophyticus, Streptococcus agalactiae, Streptococcus pneumoniae, Streptococcus pyogenes, Treponema pallidum, Vibrio cholerae, Yersinia pestis.

In one preferred embodiment the pathogenic bacteria is Legionella pneumophila.

### Methods of Treatment

Another aspect of the present invention relates to a method for treatment of an individual and/or an animal infected with pathogenic bacteria having type IB P-type ATPases in their cell membrane, said method comprising administering to said individual an inhibitor of a type IB P-type ATPase.

The individual may be a human being which is infected with one or more pathogenic bacteria. The human being may be infected with any human pathogenic bacteria.

The animal may be any kind of animal having type IB P-type ATPases in their cell membranes. In one preferred embodiment the animal is a mammal.

In one embodiment the type IB P-type ATPase is a Cu⁺ ATPase.

In one preferred embodiment the inhibitor identified according to the methods described herein does not bind human ATPases or human type IB P-type ATPases.

It is preferred that the inhibitor is identified according to the methods described herein and above.

### Administration Routes

### Systemic Treatment

The main routes of administration are oral and parenteral in order to introduce the inhibitor into the blood stream to ultimately target the sites of desired action.

Appropriate dosage forms for such administration may be prepared by conventional techniques.

### Oral Administration

Oral administration is normally for enteral drug delivery, wherein the inhibitor is delivered through the enteral mucosa.

### Parenteral Administration

Parenteral administration is any administration route not being the oral/enteral route whereby the medicament avoids first-pass degradation in the liver. Accordingly, parenteral administration includes any injections and infusions, for example bolus injection or continuous infusion, such as intravenous administration, intramuscular administration, subcutaneous administration. Furthermore, parenteral administration includes inhalations and topical administration.

Accordingly, the inhibitor may be administered topically to cross any mucosal membrane of an animal to which the biologically active substance is to be given, e.g. in the nose, vagina, eye, mouth, genital tract, lungs, gastrointestinal tract, or rectum, preferably the mucosa of the nose, or mouth, and accordingly, parenteral administration may also include buccal, sublingual, nasal, rectal, vaginal and intraperitoneal administration as well as pulmonal and bronchial administration by inhalation or installation. Also, the inhibitor may be administered topically to cross the skin.

The subcutaneous and intramuscular forms of parenteral administration are generally preferred.

### Local Treatment

The inhibitor according to the invention may be used as a local treatment, i.e. be introduced directly to the site(s) of action as will be described below.

Accordingly, the inhibitor may be applied to the skin or mucosa directly, or the inhibitor may be injected into the site of action, for example into the diseased tissue or to an end artery leading directly to the diseased tissue.

### Pharmaceutical Formulations

Whilst it is possible for the inhibitors of the present invention to be administered as the raw chemical, it is preferred to present them in the form of a pharmaceutical formulation. Accordingly, the present invention further provides a pharmaceutical formulation, which comprises a compound of the present invention or a pharmaceutically acceptable salt thereof, as herein defined, and a pharmaceutically acceptable carrier therefore. The pharmaceutical formulations may be prepared by conventional techniques, e.g. as described in Remington: The Science and Practice of Pharmacy 2005, Lippincott, Williams & Wilkins.

The pharmaceutically acceptable carriers can be either solid or liquid. Solid form preparations include powders, tablets, pills, capsules, cachets, suppositories, and dispersible granules. A solid carrier can be one or more excipients which may also act as diluents, flavoring agents, solubilizers, lubricants, suspending agents, binders, preservatives, wetting agents, tablet disintegrating agents, or an encapsulating material.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The inhibitors of the present invention may be formulated for parenteral administration and may be presented in unit dose form in ampoules, pre-filled syringes, small volume infusion or in multi-dose containers, optionally with an added preservative. The compositions may take such forms as suspensions, solutions, or emulsions in oily or aqueous vehicles, for example solutions in aqueous polyethylene glycol. Examples of oily or non-aqueous carriers, diluents, solvents or vehicles include propylene glycol, polyethylene glycol, vegetable oils (e.g., olive oil), and injectable organic esters (e.g., ethyl oleate), and may contain agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The inhibitors of the invention may also be formulated for topical delivery. The topical formulation may include a pharmaceutically acceptable carrier adapted for topical administration. Thus, the inhibitors may take the form of a suspension, solution, ointment, lotion, sexual lubricant, cream, foam, aerosol, spray, suppository, implant, inhalant, tablet, capsule, dry powder, syrup, balm or lozenge, for example.

Preferably, the formulation will comprise about 0.5% to 75% by weight of the active ingredient(s) with the remainder consisting of suitable pharmaceutical excipients as described herein.

Pharmaceutically acceptable salts of the instant inhibitors, where they can be prepared, are also intended to be covered by this invention. These salts will be ones which are acceptable in their application to a pharmaceutical use. By that it is meant that the salt will retain the biological activity of the parent compound and the salt will not have untoward or deleterious effects in its application and use in treating diseases.

Pharmaceutically acceptable salts are prepared in a standard manner. If the parent compound is a base it is treated with an excess of an organic or inorganic acid in a suitable solvent. If the parent compound is an acid, it is treated with an inorganic or organic base in a suitable solvent.

The inhibitors of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, whether by oral, rectal, or parenteral (including subcutaneous) route, in an effective amount.

Examples of pharmaceutically acceptable acid addition salts for use in the present inventive pharmaceutical composition include those derived from mineral acids, such as hydrochloric, hydrobromic, phosphoric, metaphosphoric, nitric and sulfuric acids, and organic acids, such as tartaric, acetic, citric, malic, lactic, fumaric, benzoic, glycolic, gluconic, succinic, p-toluenesulphonic acids, and arylsulphonic, for example.

### Pharmaceutical Formulations for Oral Administration

The inhibitors of the present invention may be formulated in a wide variety of formulations for oral administration. Solid form preparations may include powders, tablets, drops, capsules, cachets, lozenges, and dispersible granules. Other forms suitable for oral administration may include liquid form preparations including emulsions, syrups, elixirs, aqueous solutions, aqueous suspensions, toothpaste, gel dentrifrice, chewing gum, or solid form preparations which are intended to be converted shortly before use to liquid form preparations, such as solutions, suspensions, and emulsions.

In powders, the carrier is a finely divided solid which is a mixture with the finely divided active component. In tablets, the active component is mixed with the carrier having the necessary binding capacity in suitable proportions and compacted in the shape and size desired. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like.

Drops according to the present invention may comprise sterile or non-sterile aqueous or oil solutions or suspensions, and may be prepared by dissolving the active ingredient in a suitable aqueous solution, optionally including a bactericidal and/or fungicidal agent and/or any other suitable preservative, and optionally including a surface active agent. Suitable solvents for the preparation of an oily solution include glycerol, diluted alcohol and propylene glycol.

Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents.

### Pharmaceutical Formulations for Parenteral Administration

### Injections and Infusions

The inhibitors of the present invention may be formulated in a wide variety of formulations for parenteral administration.

For injections and infusions the formulations may take such forms as suspensions, solutions, or emulsions in oily or aqueous vehicles, for example solutions in aqueous polyethylene glycol. Alternatively, the active ingredient may be in powder form, obtained by aseptic isolation of sterile solid or by lyophilisation from solution for constitution before use with a suitable vehicle, e.g., sterile, pyrogen-free water. The formulations can be presented in unit-dose or multi-dose sealed containers, such as ampoules, vials, pre-filled syringes, infusion bags, or can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets.

Examples of oily or non-aqueous carriers, diluents, solvents or vehicles include propylene glycol, polyethylene glycol, vegetable oils, and injectable organic esters, and may contain formulatory agents such as preserving, wetting, emulsifying or suspending, stabilizing and/or dispersing agents.

The formulations for injection will typically contain from about 0.5 to about 25% by weight of the active ingredient in solution.

### Topical Delivery

The inhibitors may also be administered topically. Regions for topical administration include the skin surface and also mucous membrane tissues of the vagina, rectum, nose, mouth, and throat.

The topical composition will typically include a pharmaceutically acceptable carrier adapted for topical administration. Thus, the composition may take the form of a suspension, solution, ointment, lotion, sexual lubricant, cream, foam, aerosol, spray, suppository, implant, inhalant, tablet, capsule, dry powder, syrup, balm or lozenge, for example. Methods for preparing such compositions are well known in the pharmaceutical industry.

The inhibitors of the present invention may be formulated for topical administration to the epidermis as ointments, creams or lotions, or as a transdermal patch. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin or a fatty acid. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Lotions according to the present invention also include those suitable for application to the eye. An eye lotion may comprise a sterile aqueous solution optionally containing a bactericide.

Nasal, Pulmonary and Bronchial Administration Formulations for use in nasal, pulmonary and/or bronchial administration are normally administered as aerosols in order to ensure that the aerosolized dose actually reaches the mucous membranes of the nasal passages, bronchial tract or the lung. The term “aerosol particle” is used herein to describe the liquid or solid particle suitable for nasal, bronchial or pulmonary administration, i.e., that will reach the mucous membranes.

Typically aerosols are administered by use of a mechanical devices designed for pulmonary and/or bronchial delivery, including but not limited to nebulizers, metered dose inhalers, and powder inhalers. With regard to construction of the delivery device, any form of aerosolization known in the art, including but not limited to spray bottles, nebulization, atomization or pump aerosolization of a liquid formulation, and aerosolization of a dry powder formulation, can be used.

Liquid Aerosol Formulations in general contain a compound of the present invention in a pharmaceutically acceptable diluent. Pharmaceutically acceptable diluents include but are not limited to sterile water, saline, buffered saline, dextrose solution, and the like.

Formulations for dispensing from a powder inhaler device will normally comprise a finely divided dry powder containing pharmaceutical composition of the present invention (or derivative) and may also include a bulking agent, such as lactose, sorbitol, sucrose, or mannitol in amounts which facilitate dispersal of the powder from the device. Dry powder formulations for inhalation may also be formulated using powder-filled capsules, in particularly capsules the material of which is selected from among the synthetic plastics.

The formulation is formulated to the type of device employed and may involve the use of an appropriate propellant material, in addition to the usual diluents, adjuvants and/or carriers useful in therapy and known to the person skilled in the art. The propellant may be any propellant generally used in the art. Specific non-limiting examples of such useful propellants are a chlorofluorocarbon, a hydrofluorocarbon, a hydrochlorofluorocarbon, or a hydrocarbon.

The formulations of the present embodiment may also include other agents useful for pH maintenance, solution stabilization, or for the regulation of osmotic pressure.

The formulations of the present embodiment may also include other agents useful for pH maintenance, solution stabilization, or for the regulation of osmotic pressure.

### Transdermal Delivery

The pharmaceutical agent-chemical modifier complexes described herein can be administered transdermally. Transdermal administration typically involves the delivery of a pharmaceutical agent for percutaneous passage of the drug into the systemic circulation of the patient. The skin sites include anatomic regions for transdermally administering the drug and include the forearm, abdomen, chest, back, buttock, mastoidal area, and the like.

Transdermal delivery is accomplished by exposing a source of the complex to a patient's skin for an extended period of time. Transdermal patches have the added advantage of providing controlled delivery of a pharmaceutical agent-chemical modifier complex to the body. Such dosage forms can be made by dissolving, dispersing, or otherwise incorporating the pharmaceutical agent-chemical modifier complex in a proper medium, such as an elastomeric matrix material. Absorption enhancers can also be used to increase the flux of the compound across the skin. The rate of such flux can be controlled by either providing a rate-controlling membrane or dispersing the compound in a polymer matrix or gel. For example, a simple adhesive patch can be prepared from a backing material and an acrylate adhesive.

### Vaginal Administration

The inhibitors of the present invention may be formulated for vaginal administration. Pessaries, tampons, creams, gels, pastes, foams or sprays containing in addition to the active ingredient such carriers as are known in the art to be appropriate.

### Nasal Administration

The inhibitors of the present invention may be formulated for nasal administration. The solutions or suspensions are applied directly to the nasal cavity by conventional means, for example with a dropper, pipette or spray. The formulations may be provided in a single or multidose form. In the latter case of a dropper or pipette this may be achieved by the patient administering an appropriate, predetermined volume of the solution or suspension. In the case of a spray this may be achieved for example by means of a metering atomizing spray pump.

### Enteric Coating

When desired, formulations can be prepared with enteric coatings adapted for sustained or controlled release administration of the active ingredient.

### Dosages and Dosing Regimes

The dosage requirements will vary with the particular drug composition employed, the route of administration and the particular subject being treated. It will also be recognized by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained using conventional course of treatment determination tests.

The daily oral dosage regimen will preferably be from about 0.01 to about 80 mg/kg of total body weight, but is dependent on the type of administrative route. The daily parenteral dosage regimen about 0.001 to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day.

The Inhibitor according to the present invention is given in an effective amount to an individual in need there of. The daily parenteral dosage of inhibitor according to the present invention may in one embodiment be in the range of from about 0.01 milligram per kg body weight to about 80 milligram per kg body weight, such as from about 1 milligram per kg body weight to about 75 milligram per kg body weight, for example from about 5 milligram per kg body weight per dose to about 70 milligram per kg body weight, such as from about 10 milligram per kg body weight per dose to about 65 milligram per kg body weight, for example about 15 milligram per kg body weight per dose to about 60 milligram per kg body weight, such as from about 20 milligram per kg body weight per dose to about 55 milligram per kg body weight, for example about 25 milligram per kg body weight per dose to about 50 milligram per kg body weight, such as from about 30 milligram per kg body weight per dose to about 45 milligram per kg body weight, for example about 35 milligram per kg body weight per dose to about 40 milligram per kg body weight, for example from about 0.01 milligram per kg body weight to about 10 milligram per kg body weight, such as from about 1 milligram per kg body weight to about 9 milligram per kg body weight, for example about 2 milligram per kg body weight to about 8 milligram per kg body weight, such as from about 3 milligram per kg body weight to about 7 milligram per kg body weight, such as from about 4 milligram per kg body weight to about 5 milligram per kg body weight, for example from about 0.01 milligram per kg body weight to about 1 milligram per kg body weight, such as from about 0.02 milligram per kg body weight to about 0.09 milligram per kg body weight, for example from about 0.03 milligram per kg body weight to about 0.08 milligram per kg body weight, such as from about 0.04 milligram per kg body weight to about 0.07 milligram per kg body weight, such as from about 0.05 milligram per kg body weight to about 0.06 milligram per kg body weight,

The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

When the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the patient that corresponds to a concentration of one or more compounds according to the invention.

### EXAMPLES

### Sample Selection

Putative copper pump genes Ipg0231, Ipg1024, Ipg1626 and Ipg2691 from Legionella pneumophila were cloned into pET22b(+) and checked for expression. LPG0231, LPG1024 and LPG1626 proteins were purified by nickel affinity and size-exclusion chromatography and then tested in crystallization experiments. LPG1024 provided promising hit conditions.

### Sample Preparation

A pET22b(+) construct containing the full length Ipg1024 gene was transformed into C43 E. coli cells. The cells were grown to OD_{600 nm}=0.5-0.8 in LB medium, induced with 1 mM IPTG, harvested after 16 hours culture and then resuspended in 50 mM Tris-HCl, pH7.6, 200 mM KCl, 20% glycerol and frozen at −20° C. A construct without the heavy metal binding domain (lacking the first 74 amino acid residues) was grown and purified in the same manner as the full-length construct. Selenomethionine derivatized protein was produced from the same E. coli strain and construct in a minimal medium, including 50 μg/mL L-SeMet. Washed cells from a preculture were diluted to OD_{600 nm}=0.8, adapted to 20° C. for one hour in the shaker and then induced with 1 mM IPTG for 16 hours. Before breakage of cells with native or selenomethionine derivatized protein, 5 mM of fresh β-mercaptoethanole (BME), 1 mM phenylmethylsulfonyl fluoride, 2 μg/mL DNase I and Roche protease inhibitor cocktail (1 tablet for 8 L cells) were added to the cells. Cells were opened with a high pressure homogenizer by three runs at 15-20.000 psi and kept at 4° C. throughout the purification until crystallization. Large aggregates were removed by centrifugation at 20.000×g for 45 min. Membranes were isolated by centrifugation at 250.000×g for 3 hours. Membranes were resuspended in 15 mL/g in 20 mM Tris-HCl, pH=7.6, 200 mM KCl, 20% glycerol, 5 mM BME and 1 mM MgCl₂. Membrane proteins were solubilized by addition of solid octaethylene glycol monododecyl ether (C₁₂E₈) to a 18.6 mM final concentration. Solubilization was performed by gentle stirring for 60 minutes. Unsolubilized material was removed by ultracentrifugation for 1 h at 250.000×g. Solid KCl was added to solubilized membrane solution to a final concentration of 500 mM, imidazole was added to a final concentration of 50 mM and the solution was mixed with preequilibrated Ni²⁺ beads and incubated for at least 1 hour.

After packing the beads, the beads were washed with 20 mM MOPS-KOH pH=7.4, 200 mM KCl, 20% glycerol, 5 mM BME, 1 mM MgCl₂ and 0.28 mM C₁₂E₈. Bound proteins were eluted by two gradients, from 0 to 250 and then to 500 mM imidazole. Alternatively the supernatant was incubated with approx. 20 to 25 ml pre-equilibrated Ni-NTA and incubated for at least 60 min. The Ni-NTA bound LpCopA was packed into a XK16 column (GE Healthcare) and washed with approx. 5 CV wash buffer [20 mM MOPSKOH pH 7.4, 200 mM KCl, 20% glycerol, 5 mM BME, 1 mM MgCl2, 0.28 mM C12E8]. LpCopA was eluted by a two-step gradient (6 column volumes (CV) to 15% elution buffer (75 mM Imidazole), 2 CVs to 100% elution buffer). The elution buffer is identical to the wash buffer, but has 500 mM imidazole in addition.

Eluted protein was checked by SDS-PAGE and the fractions containing CopA were pooled and concentrated to around 20-25 mg/mL. Then, typically 5-7 mg concentrated protein was applied to a Superose 6 size-exclusion column equilibrated in 20 mM MOPS-KOH pH=6.8, 80 mM KCl, 20% glycerol, 5 mM BME, 1 mM MgCl₂ and 0.28 mM C₁₂E₈, here coined Buffer A. The fractions containing CopA were pooled and concentrated to 20-25 mg/mL, flash frozen in aliquots of 200 μL in liquid nitrogen and stored at −80° C. For final samples, aliquots were diluted to 10 mg/mL in Buffer A and re-lipidated for 16 hours with saturating amounts of dioleoyl-phosphatidylcholine lipid using an additional approximately 0.5 mg C₁₂E₈ (the amount varied for every protein preparation) per 100 μL CopA.

### Functional Characterization

The LpCopA (Legionella pneumophila Cu⁺ transporting P-type ATPase) ATPase activity was measured by the Baginsky method with Bismuth detection (see example 3). In a total volume of 50 μL, 15 μg (4 μM final concentration) of CopA was mixed with 40 mM MOPS-KOH pH=6.8, 150 mM NaCl, 5 mM KCl, 5 mM MgCl2, 20 mM (NH4)₂SO₄, 1 mg/mL E. coli total lipid extract, 3.7 mM C₁₂E₈, 20 mM cysteine, 5 mM NaN₃ and 0.25 mM Na₂MoO₄. 5 mM ATP was added to start the reaction and the mixture was incubated at 37° C. for 15 minutes. 75 μL of freshly prepared solution (2.86% Ascorbic acid, 1M HCl, 0.48% (NH₄)₂MoO₄, 2.86% SDS) was added to stop the reaction and start color development. After 8 minutes incubation on ice, 125 μL of 3.5% Bismuth citrate, 1 M HCl and 3.5% sodium citrate was added to the mixture and incubated for another 30 minutes at 19° C. Absorbance was measured at 710 nm. For the HMBD-truncated construct the final protein concentration was 12 μM.

### Example 1

### LpCopA Complexed with AlF₄⁻

### Crystallization

Prior to the crystallization experiments the sample was ultracentrifuged for 10 minutes at 100.000×g, the supernatant treated with 10 mM NaF, 2 mM AlCl₃, 2 mM EGTA, 10 μM ammonium-tetrathiomolybdate (TTM) as well as the secondary detergents Cymal-6 or deoxy Big CHAP (both between 3-5×CMC, except for seleno-methionine derivatized protein that required lower concentrations).

Crystals were grown at 19° C. using the hanging drop vapor diffusion method with a reservoir solution containing 6% (w/v) PEG6000, 10% (v/v) Glycerol, 140 mM NaCl, 3% v/v t-BuOH, 5 mM BME. 1 μL protein and 1 μL precipitant were mixed and crystals from these drops appeared within 1 week and developed to full size within 4 weeks. Optimal crystals could only be obtained following multiple rounds of optimization using same batch aliquots. About 1 g of protein was prepared through this project and more than 2000 crystals tested at synchrotrons (about half soaked with heavy-metal compounds). Full size crystals (20×80×300 μm³) were mounted in Litholoops (Molecular Dimensions) and flash-frozen in liquid nitrogen. Complete native data were collected at the SLS X06SA beam line taking advantage of a PILATUS 6M detector. For the K₂Pt(CN)₄, Na₃IrCl₆ and P-chloromercury Benzoic acid derivatives concentrated stock solutions were added to the crystals at a final concentration of approximately 1 mM. For Ta₆Br₁₂, powder and 100 mM MOPS-KOH, pH=7.4 were added to the crystals.

### Data Collection and Processing

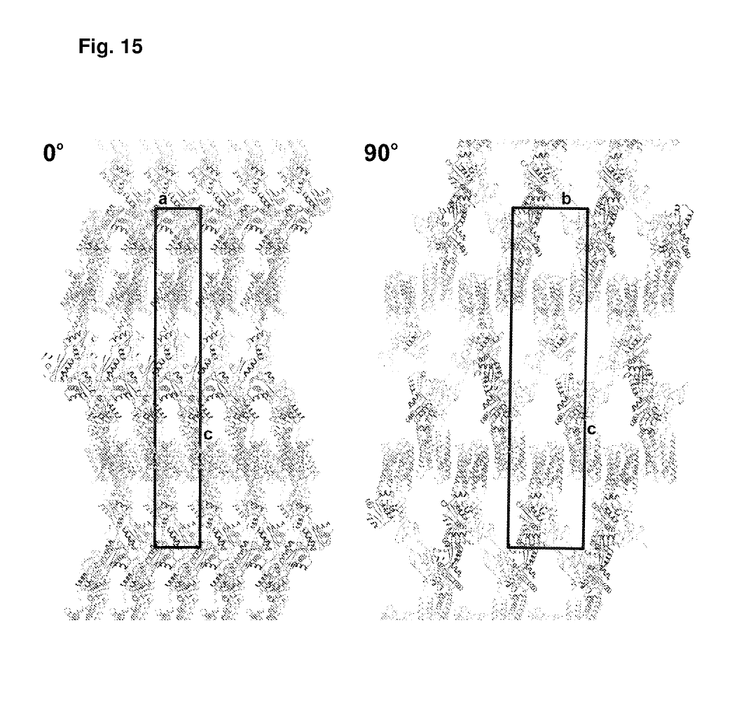

Data were processed and scaled with XDS (Kabsch, W., Journal of Applied Crystallography 26, 795 (1993)). The crystals belonged to space group P1 with unit cell parameters a=44.1 Å, b=72.9 Å, c=329.6 Å with four CopA monomers in the unit cell related by a strong P 2₁2₁2, pseudosymmetry (Table 8 and FIG. 15). Initial phases were obtained by a procedure exploiting semi-automatic screening of more than 5000 molecular replacement runs (in space group P 2₁2₁2₁) using PHASER (Storoni, L. C., McCoy, A. J., and Read, R. J., Acta CrystallogrD Biol Crystallogr 60 (Pt 3), 432 (2004)) with systematic combination of data sets, partial models (derived from SERCA1a structures and CopA soluble domains), and search parameters, exploiting previously described rationales (Pedersen, B. P., Morth, J. P., and Nissen, P., Acta CrystallogrD Biol Crystallogr 66 (Pt 3), 309 (2010)). Initial Pt and Ir heavy-atom sites were pinpointed in anomalous difference Fourier maps using the molecular replacement phases. After this the molecular replacement phases were discarded and the HA-site coordinates used to calculate and refine unbiased experimental phases by MIRAS in SHARP (Vonrhein, C., Blanc, E., Roversi, P., and Bricogne, G., Methods Mol Biol 364, 215 (2007)). The MIRAS phases were further refined and extended to 3.3 Å resolution in RESOLVE (Terwilliger, T. C., Acta Crystallogr D Biol Crystallogr 56 (Pt 8), 965 (2000)) using solvent flattening, histogram matching and NCS averaging. The model was built in Coot (Emsley, P., Lohkamp, B., Scott, W. G., and Cowtan, K., Acta CrystallogrD Biol Crystallogr 66 (Pt 4), 486 (2010)) and 0 (Jones, T. A., Zou, J. Y., Cowan, S. W., and Kjeldgaard, M., Acta Crystallogr A 47 (Pt 2), 110 (1991)) using as templates the corresponding SERCA1a structure (Olesen, C. et al., Nature 450 (7172), 1036 (2007)), the A. fulgidus CopA N- and P-domains structure (Tsuda, T. and Toyoshima, C., EMBO J 28 (12), 1782 (2009)) and the A. fulgidus CopA A-domain structure (Sazinsky, M. H., Agarwal, S., Arguello, J. M., and Rosenzweig, A. C., Biochemistry 45 (33), 9949 (2006)) to guide chain tracing. Model refinement was performed in phenix.refine (Adams, P. D. et al., Acta Crystallogr D Biol Crystallogr 66 (Pt 2), 213). R factors and model refinement did not improve (R_{free}>40%) until the space group was changed from P2₁2₁2₁ to P1 which gave the only obvious clue to the correct P1 symmetry. The final model yielded a crystallographic R-factor of 23.6% and a free R-factor of 26.3%. The R_{free} set was picked in resolution bins to reduce NCS-bias. Molprobity (Chen, V. B. et al., Acta CrystallogrD Biol Crystallogr 66 (Pt 1), 12) evaluation of the Ramachandran plot displayed 98.8% in allowed regions (89.6% in favored regions) and 1.2% in disallowed regions. All figures were prepared using Pymol (DeLano, W. L., Curr Opin Struct Biol. 2002 February; 12(1):14-20).

### Protein Characterization and Structure Determination

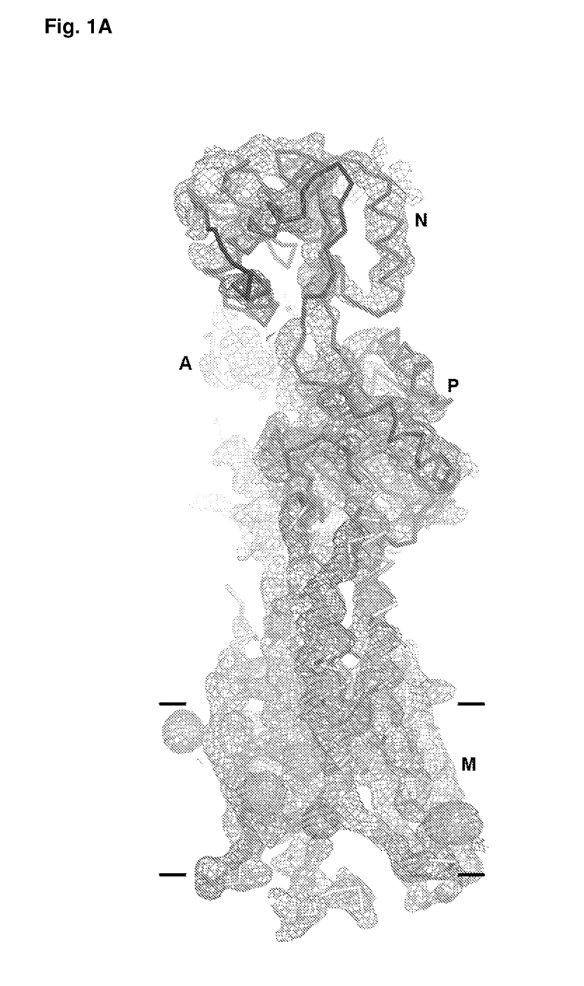

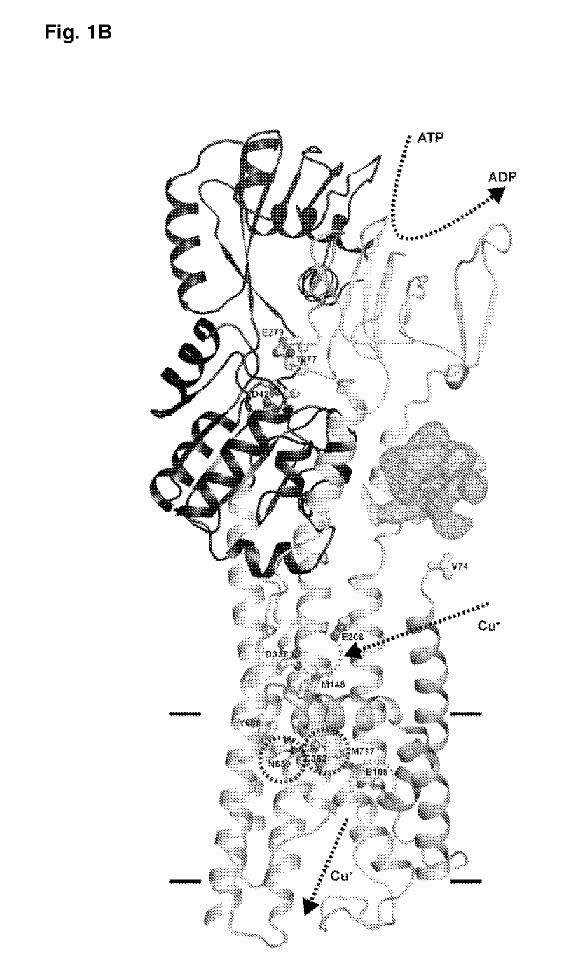

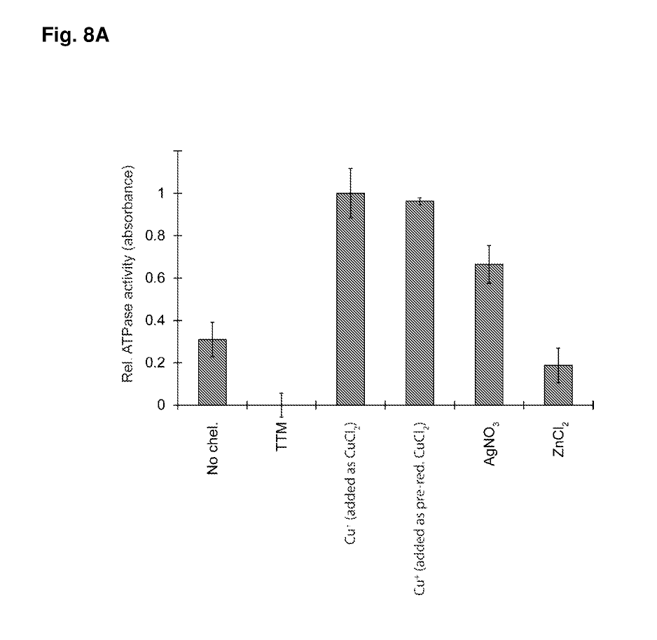

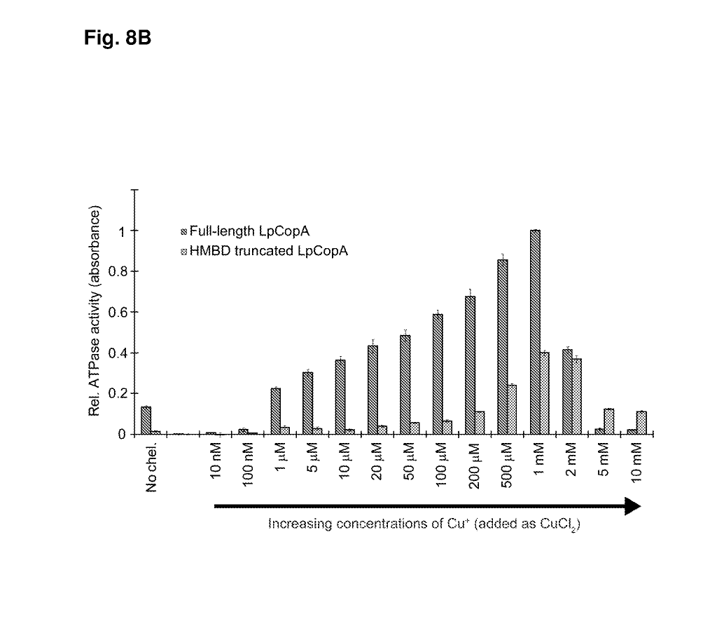

The crystal structure was derived from the Legionella pneumophila gene LPG1024 (SEQ ID NO: 1). LPG1024 shows significant sequence identity to e.g. CopA from Archaeoglobus fulgidus (39%)¹⁸, and to the human ATP7A and ATP7B (37% and 38% sequence identity, respectively, for the core enzyme) that all conduct active efflux of Cu⁺ ions (FIGS. 7A and 7C). Indeed, we observe a copper-induced ATPase activity of the isolated protein (FIG. 8), which is referred to as LpCopA or CopA. The protein was crystallized in the presence of high amounts of exogenous lipid solubilized by detergent (Methods), and the structure was determined from unbiased electron density maps obtained by multiple isomorphous replacement with anomalous diffraction (MIRAS) and density modification (FIG. 1A and Table 8). Model building was facilitated by anomalous difference Fourier maps derived from crystals of selenomethionine protein, pinpointing 22 (out of 27 possible) SeMet positions (FIG. 1A) and template structures from cytoplasmic domain and further guided by SERCA1a in the equivalent state. The final model includes residues Va174 to the C-terminal Leu736. The unmodelled 73 N-terminal residues encompass a single HMBD, which is however partially visible as low resolution electron density features (see later). The final model yields an R_{work} of 23.6% and R_{free} of 26.3% at 3.2 Å resolution in the space group P1 with four-fold non-crystallographic symmetry (Table 8).

### Overall Architecture of CopA Confirms the P-Type ATPase Fold

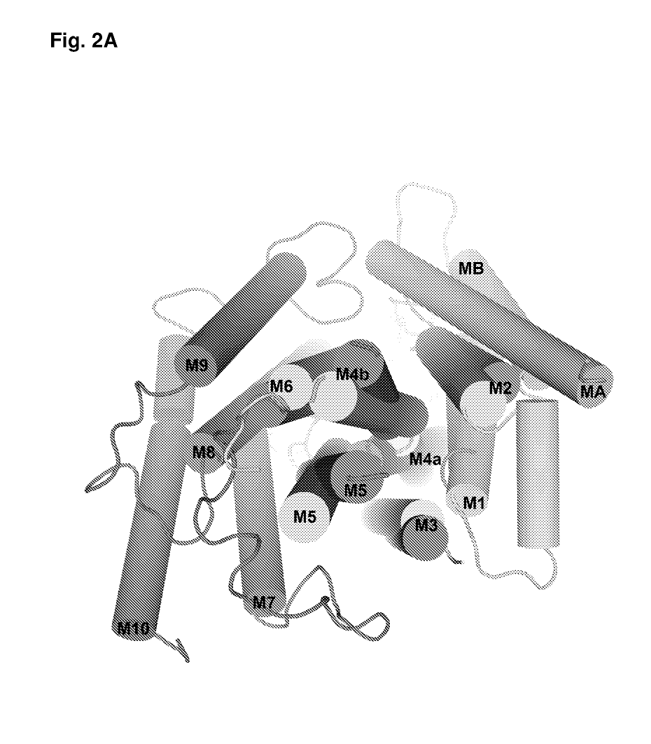

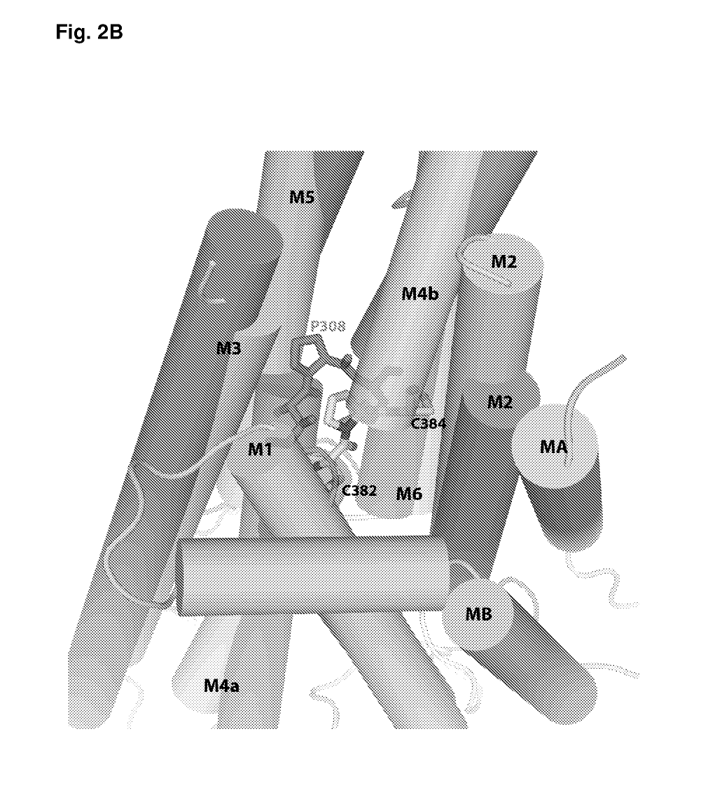

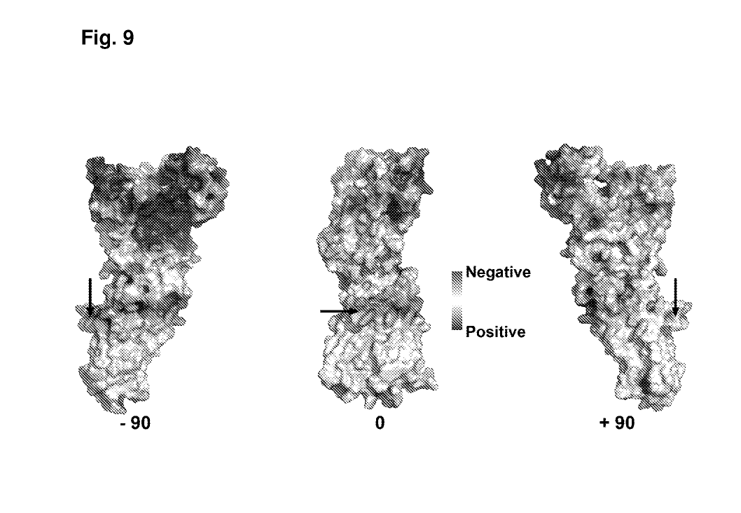

The cytosolic part of CopA displays three domains that constitute the characteristic headpiece of the P-type ATPases: the A-domain (actuator, residues 211-331), P-domain (phosphorylation, residues 405-429 and 554-673) and the N-domain (nucleotide binding, residues 430-553 inserted into the P-domain) (FIG. 1B). The M-domain comprises eight transmembrane segments: two N-terminally located helices, denoted MA and MB, followed by six helices, M1 through M6 (FIG. 2A). Compared to the previously determined structures of P-type ATPases, the A-domain is truncated at the N-terminal part and appears only as the insert between M2 and M3. MA interacts with M2 and M6, and MB interacts with M1 and M2. MB consists of two helical segments, a transmembrane helix followed by a kink and an amphipathic helix positioned at the cytoplasmic membrane interface (FIG. 1B and FIG. 9).

### CopA in the E2-P Transition State

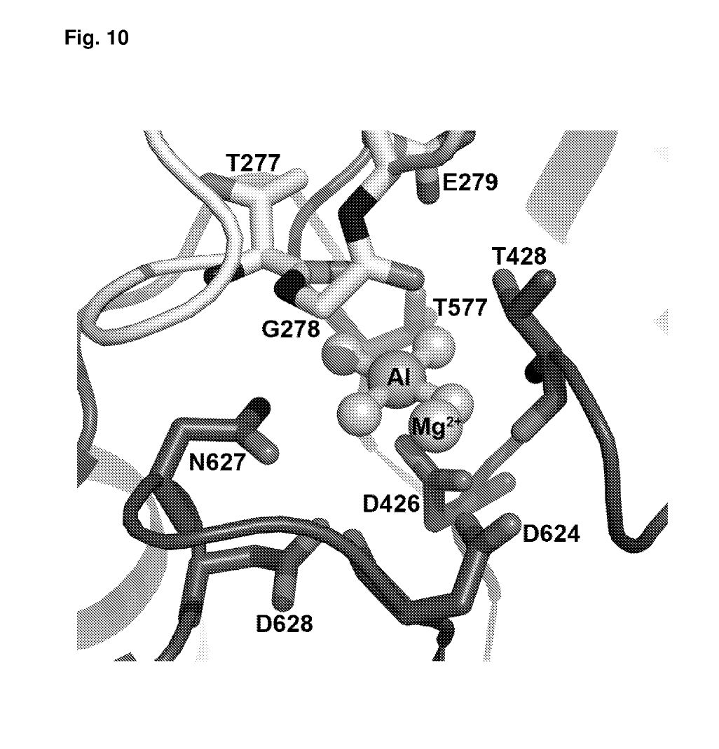

LpCopA was crystallized in complex with AlF₄⁻ in an occluded, copper-released E2-P transition state of dephosphorylation. Indeed, a comparison to SERCA1a structures shows the closest resemblance to the proton-occluded E2-P transition state with a similar configuration of domains that supports the docking of the conserved TGE loop of the A-domain (residues 277-279, associated with dephosphorylation) to the phosphorylation site at the P-domain. The AlF₄⁻ transition state analog coordinates the side chain of the Asp426 phosphorylation site (of the conserved DKTGT motif), and also interacts with main and side chain oxygens of Thr428, Thr577, Asn627 and Asp628 (FIG. 10). Furthermore, AlF₄⁻ is coordinated by an Mg²⁺ ion which is also associated with oxygens from the main chain of Thr428 and side chain of Asp624. Gly278 and Glu279 of the TGE loop may activate a water molecule for dephosphorylation as observed in the equivalent state of SERCA1a, although the Glu279 residue is slightly off-set in the LpCopA structure.

The M-domain of CopA adopts a compact configuration compared to SERCA1a (pdb-id 3b9r). The CPC motif of M4 is shifted more than 4 Å towards M1-2 (and MA and MB) (FIG. 2B) and combined with a change of the M3 angle relative to the membrane, the distance between M3 and M4 narrows at the extracellular interface. The absence of the M7-M10 domain found in SERCA1a allows the entire M5 helix of CopA to approach M4 where a particular kink of M5 interacts with the CPC motif.

### The Transmembrane Metal Ion Binding Sites

The ion binding sites in P-type ATPases are typically denoted I and II, with site II being accessible through an N-terminal, cytoplasmic pathway and site I more deeply buried towards M6 (FIG. 1B). Mutagenesis in conjunction with biochemical studies have previously suggested that six invariant residues in M4, M5 and M6 of CopA contribute to two ion binding sites at the M-domain (Gonzalez-Guerrero, M. and Arguello, J. M., Proc Natl Acad Sci USA 105 (16), 5992 (2008)). Five of these, namely Cys384 (last of the CPC motif in M4), Tyr688 (M5), Asn689 (M5), Met717 (M6) and Ser721 (M6), overlap fairly well with the calcium coordinating residues of SERCA1a in the similar calcium-released state (FIG. 2C). In addition, Cys382 (first of the CPC motif in M4) replaces IIe307 in SERCA1a for which the main chain oxygen assists in calcium coordination at site II. Assuming that similar conformational changes occur in CopA as in SERCA1a, it is likely that the ion binding sites in the copper bound states bear resemblance to the calcium-bound states observed for SERCA1a. Only Tyr688 and Asn689 in M5 would require side-chain rearrangements to reach similar sites II and I, possibly assisted by Pro694 of M5 which is conserved in the class IB. Worth noting, except for the proline at M4 (part of the CPC motif) none of the residues involved in ion binding in class II ATPases are conserved in the class IB Cu⁺-ATPases. Furthermore, the only conserved and charged residue in the M-domain of LpCopA is Glu189 in M2, close to the extracellular side (see later). We take the lack of charged amino acids at the ion binding sites in CopA to indicate that it operates without counter transport, which has also never been demonstrated for a class IB P-type ATPase. In SERCA1a proton counter transport is required for charge stabilization in the occluded, calcium-free E2 states.

### The Heavy Metal Binding Domain

Key questions concern the function and localization of the N-terminal Cu⁺-binding domain (a HMBD) positioned before the MA helix in LpCopA.

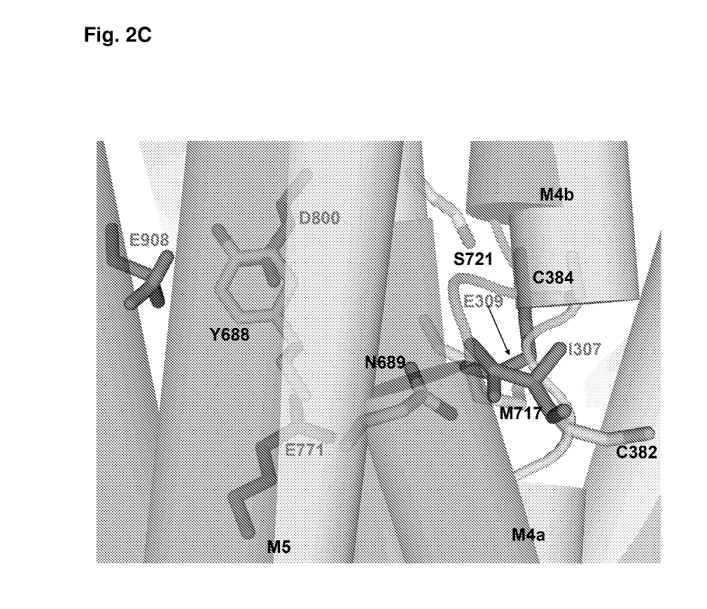

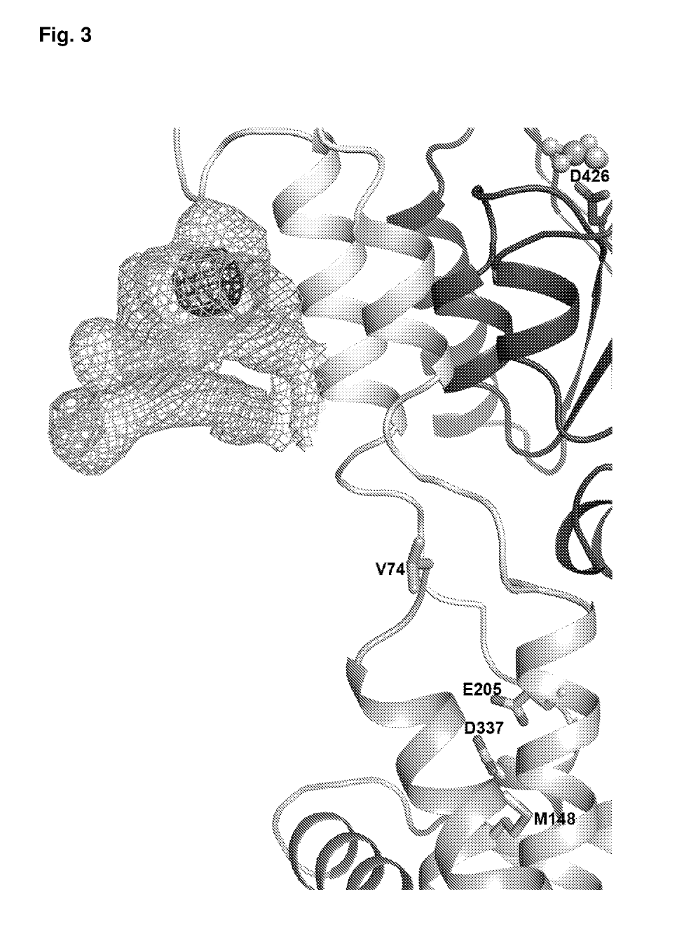

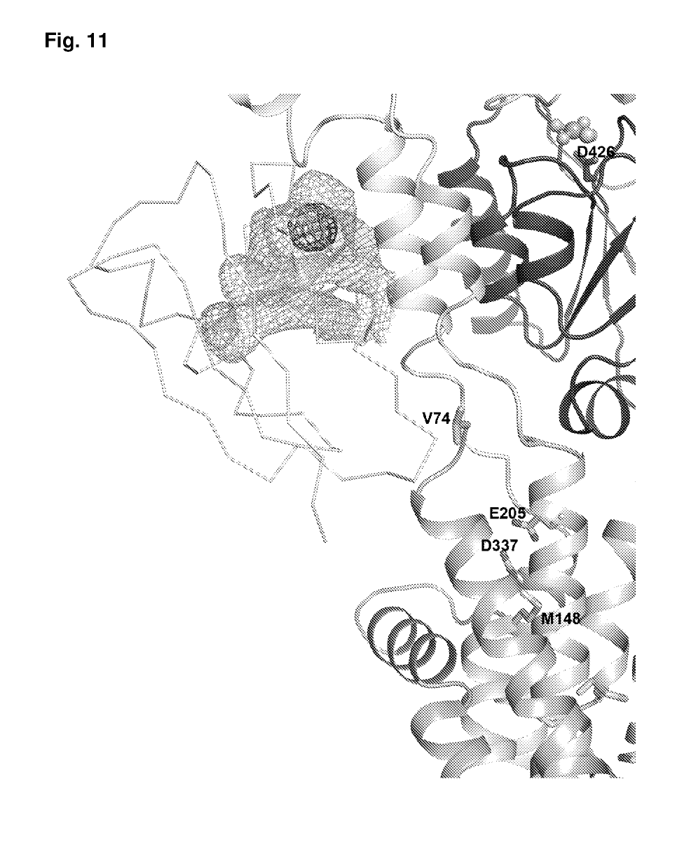

The datasets reveal continuous and coinciding electron density, located peripheral to the two small helices of the A-domain at about 15 Å from Va174. This is assigned to part of the HMBD (FIG. 3 and FIG. 11). A significant selenomethionine peak (at 5.5 σ in the anomalous difference Fourier map) overlaps with this region (FIG. 3), and likely stems from one of the four methionines found in the unmodelled HMBD.

In a further effort to exploit this observation, crystals were soaked with copper and silver compounds, which however resulted in severely impaired diffraction. The CXXC motif of the HMBD may bind e.g mercury, cadmium, lead, platinum or gold, which on the other hand would not stimulate the copper-specific binding site at the center of the occluded M-domain.

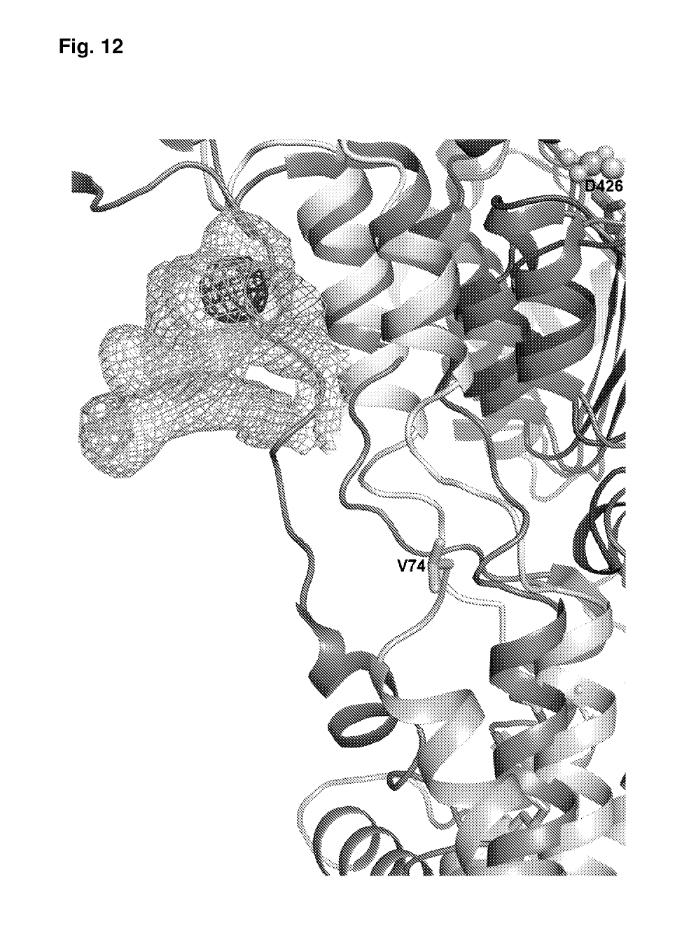

Noteworthy, the assigned position for the HMBD coincides with the linker between M1 and the N-terminal part of the A-domain in SERCA1a, which is missing in CopA (FIG. 12). The integrity of the A-M1 linker is essential for conformational changes associated with the functional cycle of SERCA1a. Similarly, the HMBD might serve a role to regulate the CopA function through interactions with the A-domain.

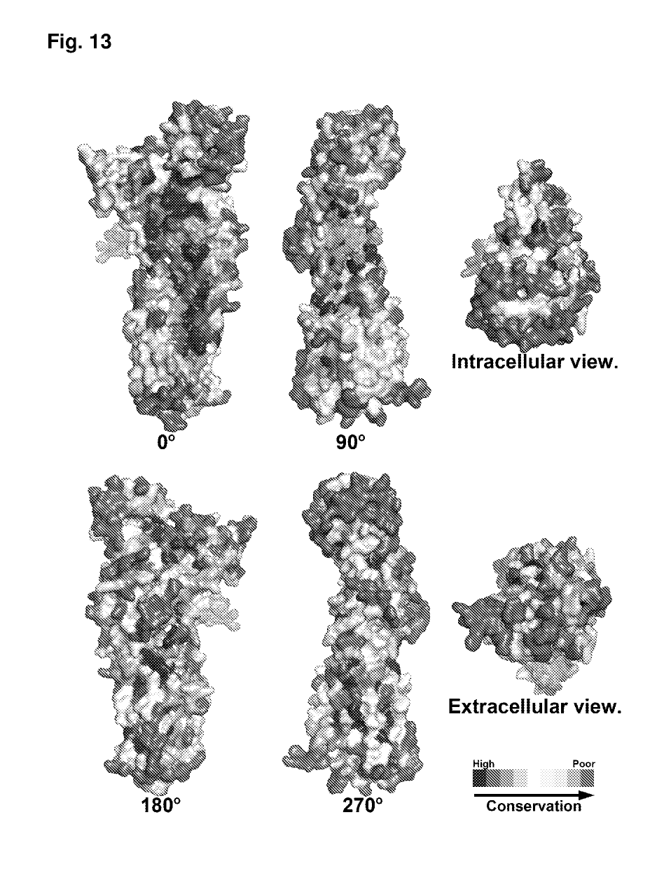

However the HMBD may (also) interacts elsewhere. Analysis of the sequence conservation of CopA proteins reveals that an entire side of their surfaces is highly conserved (FIG. 13). Surface-exposed residues are generally less conserved, unless they form binding interfaces. It is possible that this surface allows for HMBD binding and/or for dimerization (obstructed in our crystals), although it should be noted that SERCA1a displays a similar pattern. The conserved surface area is located at about the same distance from Va174 as the mapped position for the HMBD described above, and it overlaps with another of the three positions suggested by electron microscopy, at the P and N domain interface (Wu, C. C., Rice, W. J., and Stokes, D. L., Structure 16 (6), 976 (2008)). It is possible that the HMBD exhibits multiple conformations—from an inhibiting state located at the conserved surface of the P-domain (which is physically blocked by crystal contacts) to the position mapped by our crystal form.

### The Membrane Platform

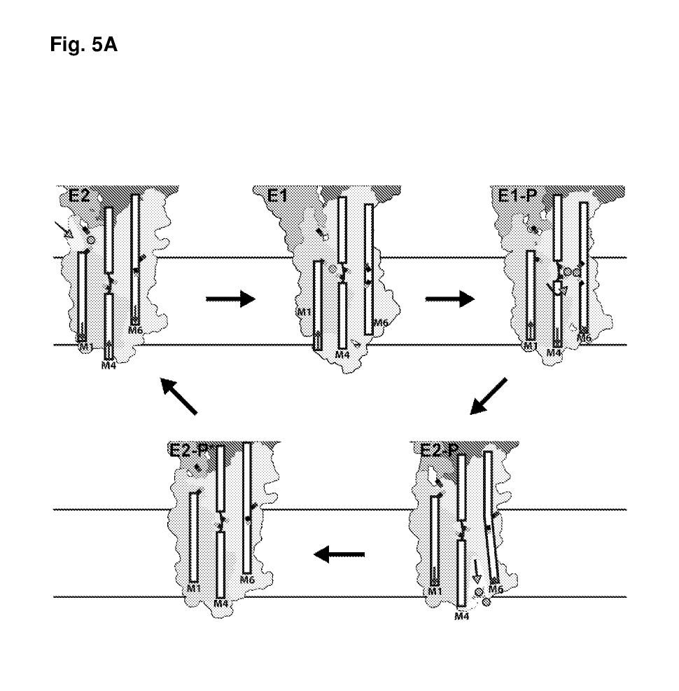

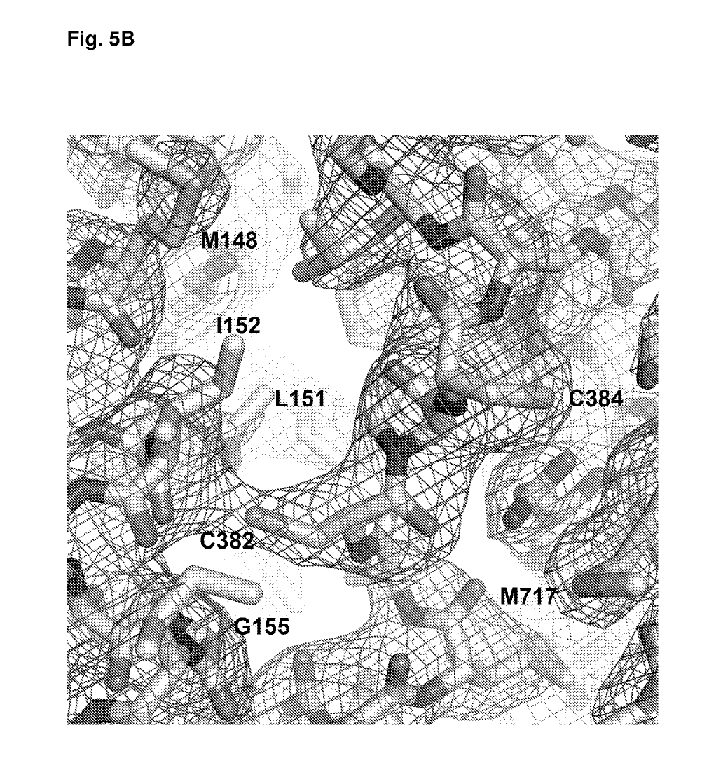

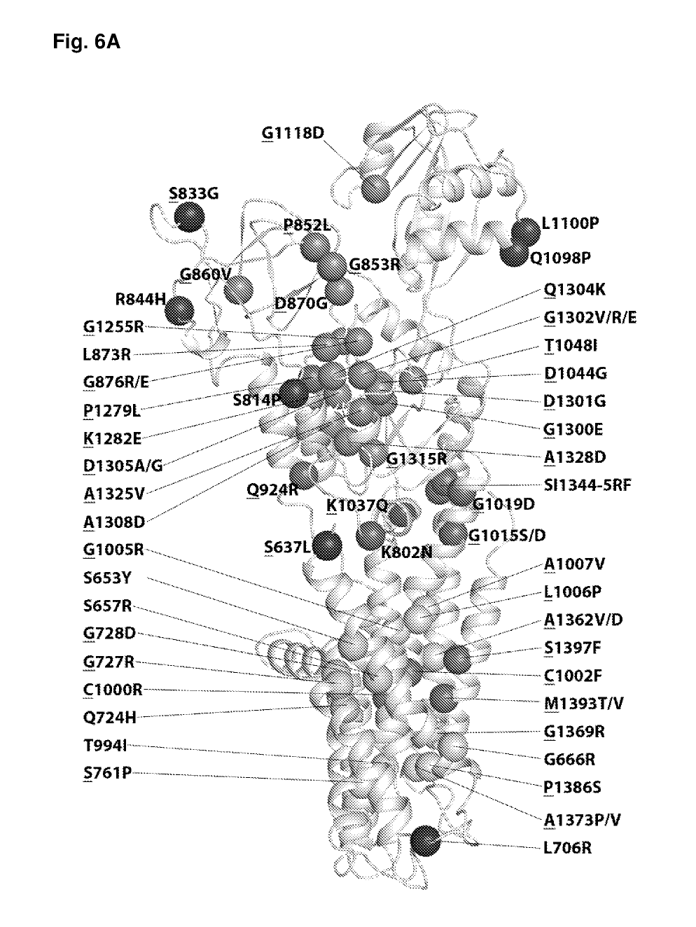

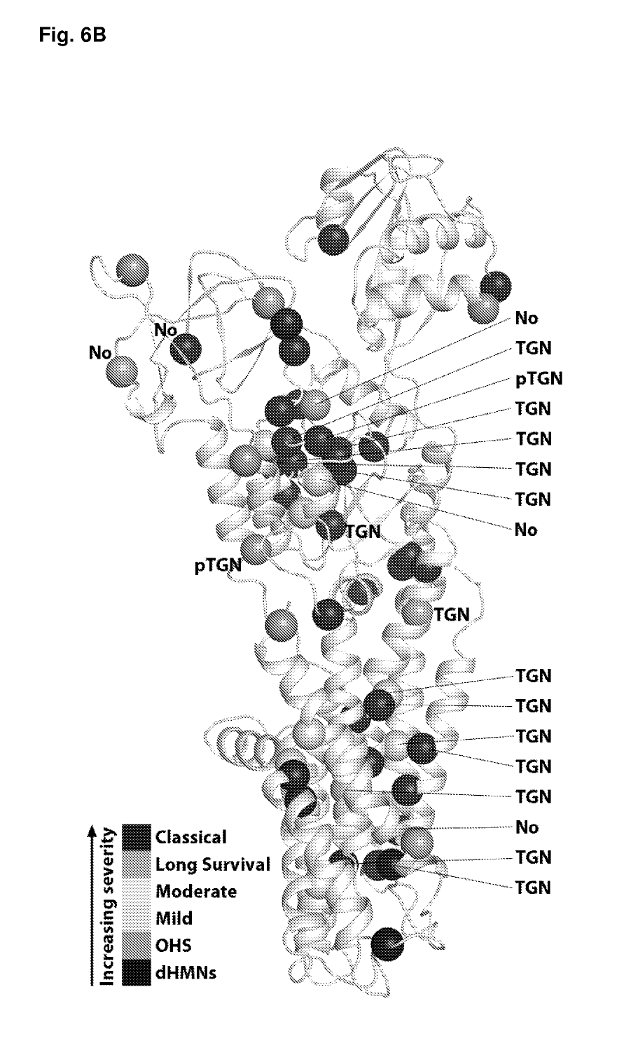

The IB-specific MA and MB helices are of special interest. MA is about 40 Å in length with a curved appearance, going from the first residue in the model, Va174 (adjacent to the A-domain and the proposed positions of the HMBD), and through the membrane. Following MA, the N-terminal part of MB provides a short transmembrane helix which kinks at the cytosolic membrane interface, facilitated by two sequential glycines Gly129 and Gly130 (FIG. 5A). The C-terminal part of MB forms an amphipathic helix with Trp131, Phe133, Phe134, Trp138 and Va1141 directed towards the membrane and Lys135, Arg136 and Lys142 facing the cytoplasm. The consensus sequence of CopA pumps preserves this GG kink motif and the highly amphipathic nature of MB (FIGS. 7A and 7C).

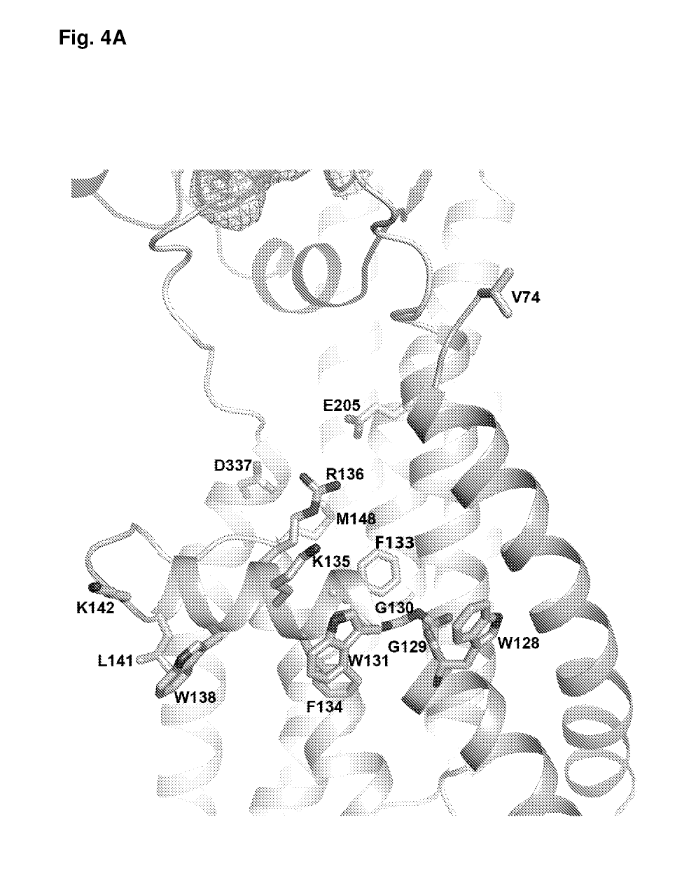

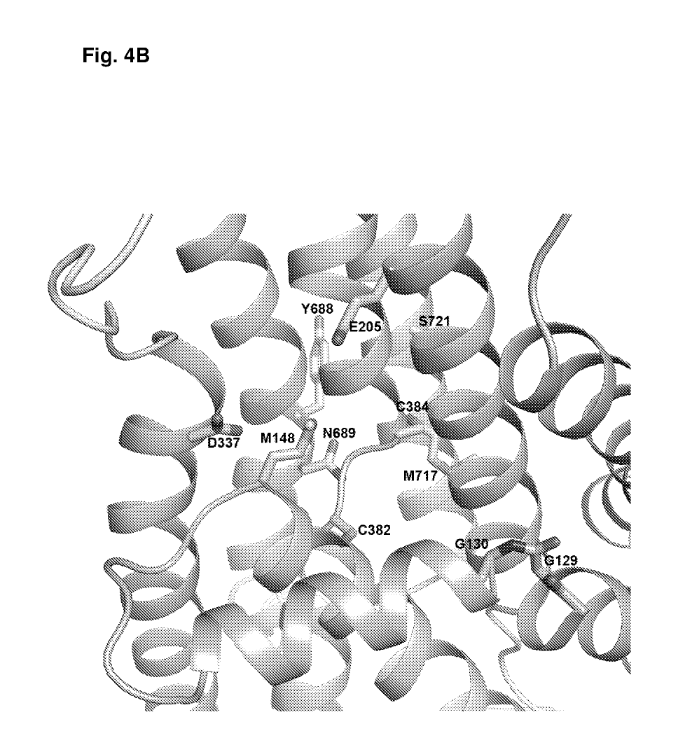

The amphipathic part of MB along with M1 (FIG. 4A) forms a platform. By analogy to SERCA1a, where the M1 region lines the putative calcium entry pathway leading to Glu309 at calcium site II (FIG. 2C), we suggest that the platform is part of a copper entry pathway. Examination of exposed residues of the platform region unveils three candidate residues that could assist in copper coordination: Met148, Glu205, and Asp337 (FIG. 4B), which are highly conserved in CopA proteins. Met148 and Asp337 are located at about 5 Å from each other (sulfur to oxygen of the side chains) whereas Glu205 is positioned a bit further away (about 7.5 Å).

### A Mechanism for Copper Transfer

The platform may provide a docking site for the HMBD (within reach) or a soluble copper chaperone, either for copper delivery and perhaps also for CopA autoregulation by the HMBD preventing soluble copper chaperones access to the M-domain. Copper may initially be transferred from the HMBD or a soluble copper chaperone to Met148 and Asp337-G1u205, conceivably in the E2 state which follows the state represented by our structure (FIG. 5A). For ion delivery to site II the tight configuration of the transmembrane helices and the orientation of the side chain of Cys382 may be of significance. In contrast to Cys384 (also site II), it points away from the suggested metal ion binding sites, towards the platform with a sulfur-to-sulfur distance to Met148 of 9.5 Å. In SERCA1a the E2 to E1 transition is associated with movement of M1 towards the extracellular side and M4 towards the cytoplasm, which would significantly increase the distance between Met148 and the carboxylic acids (G1u205 and Asp337) and reduce the distance between Met148 and Cys382. Thus, this alteration alone could assist in both lowering the affinity at the site formed by Met148, Asp337 and Glu205, and in Cu⁺ transfer, mediated by Met148, to Cys382. Concomitantly, the side chain of Cys382 must flip and possibly bring the copper ion along to site II, facilitated by a simultaneous rotational shift of M4 (as known for SERCA1a) and the flexibility around the CPC motif, to help establish the high-affinity transmembrane ion binding sites I and II.

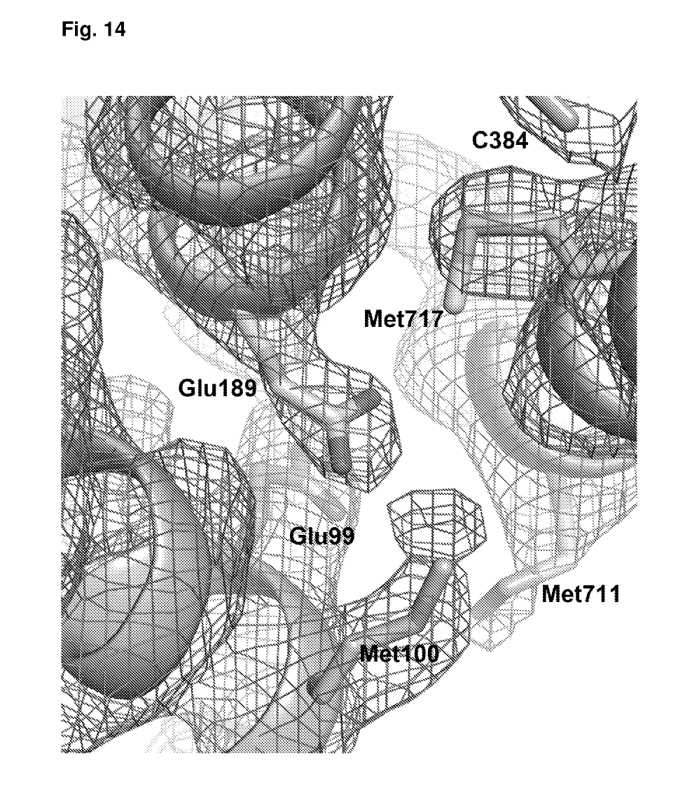

An intriguing question is how CopA releases copper from high affinity sites in the M-domain. The crystal of the present invention represents the copper released state, and the way Cys382 is oriented might be the key point also for this (FIG. 5B). Space is permitted by the highly conserved Gly155 to allow the Cys382 side chain to be directed away from the ion-binding site, stabilized by hydrogen bonds to the backbone carbonyls of Leu151 (˜2.9 Å) and possibly IIe152 (˜3.5 Å). This buried and stabilized position associated with the E2P state drives the removal of the Cys382 side chain from the high affinity transmembrane ion binding site, stimulating the E1P to E2P transition and Cu⁺ release (at least from site II) to the extracellular side. Release is likely to be further stimulated by the conserved, negatively charged residue, Glu189. Difference electron density is also observed adjacent to Glu189 which may represent a partially occupied cation site associated with the exit pathway (FIG. 14).

### Example 2

### LpCopA Complexed with BeF₃⁻

### Crystallization

Prior to the crystallization experiments the sample was ultracentrifuged for 10 minutes at 100.000×g, the supernatant treated with 10 mM NaF, 2 mM BeSO₄, 2 mM EGTA, 10 μM ammonium-tetrathiomolybdate (TTM) as well as the secondary detergents

Crystals were grown at 19° C. using the hanging drop vapor diffusion method with a reservoir solution containing 10% glycerol, 200 mM KCl, 3% t-BuOH, 14% PEG 2K MME and 5 mM BME. 1 μL protein and 1 μL precipitant were mixed and best crystals (100 μm×20 μm×20) μm for the BeF₃− conformational state were found after 2 days by the addition of 5 mM β-NAD to the protein solution. Optimal crystals could be obtained following multiple rounds of optimization using same batch aliquots.

### Data Collection, Structure Determination and Analysis

Initial screening of diffraction for different obtained protein crystals have been done at several synchrotrons (MaxLab, SLS, ESRF) and best data has been collected for both crystals at ID29 at ESRF using a pixel detector (Pilatus 6M). The beryllium fluoride LpCopA was collected with an exposure time of 0.1 s, 49.7% beam transmission and an oscillation angle increment of 0.5°. The crystal was rotated over 500°. The detector distance was 500.075 cm and the wavelength was 0.9763 Å. The aluminium fluoride LpCopA was collect with an exposure time of 0.25 s, a transmission of 17.87% and an oscillation angle increment of 0.25°. The crystal was rotated over 360°. The detector distance was set to 831.374 cm and the wavelength was at 0.9763 Å.

Data has been processed, integrated and scaled using the program package XDS (Kabsch, W. (2010), Xds. Acta Crystallogr D Biol Crystallogr 66, 125-32; Kabsch, W. (2010), Integration, scaling, space-group assignment and post-refinement. Acta Crystallogr D Biol Crystallogr 66, 133-44). while for the beryllium fluoride LpCopA structure all frames were used, for the aluminium fluoride structure only 900 frames have been used. The input file was used as defaults expect for WFAC1 that was set to 0.7 instead of 1, the beam divergence and reflection range was adapted as suggested by XDS.

Initial phases for BeF₃⁻ (E₂P mimicry) and MgF₄²⁻ (E₂;Pᵢ mimicry) conformations have been found using Phaser (McCoy, A. J., Grosse-Kunstleve, R. W., Adams, P. D., Winn, M. D., Storoni, L. C. & Read, R. J. (2007), Phaser crystallographic software. J Appl Crystallogr 40, 658-674.) provided by Phenix (Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., McCoy, A. J., Moriarty, N. W., Oeffner, R., Read, R. J., Richardson, D. C., Richardson, J. S., Terwilliger, T. C. & Zwart, P. H. (2010), PHENIX: a comprehensive Python-based system for macromolecular structure solution. Acta Crystallogr D Biol Crystallogr 66, 213-21.) using the copper-free occluded state of LpCopA28. Autobuild (Phenix package) was used for initial map improvement and initial building. Afterwards, iterative refinement and manual building was performed by Phenix and the molecular graphics program Coot (Adams, P. D., Afonine, P. V., Bunkoczi, G., Chen, V. B., Davis, I. W., Echols, N., Headd, J. J., Hung, L. W., Kapral, G. J., Grosse-Kunstleve, R. W., McCoy, A. J., Moriarty, N. W., Oeffner, R., Read, R. J., Richardson, D. C., Richardson, J. S., Terwilliger, T. C. & Zwart, P. H. (2010), PHENIX: a comprehensive Python-based system for macromolecular structure solution. Acta Crystallogr D Biol Crystallogr 66, 213-21; Emsley, P., Lohkamp, B., Scott, W. G. & Cowtan, K. (2010), Features and development of Coot. Acta Crystallogr D Biol Crystallogr 66, 486-501). Structures were analysed using Molprobity (Chen, V. B., Arendall, W. B., 3rd, Headd, J. J., Keedy, D. A., Immormino, R. M., Kapral, G. J., Murray, L. W., Richardson, J. S. & Richardson, D. C. (2010), MolProbity: all-atom structure validation for macromolecular crystallography. Acta Crystallogr D Biol Crystallogr 66, 12-21) and pictures have been generated using Pymol (Schrodinger, LLC. (2010), The PyMOL Molecular Graphics System, Version 1.3r1). The Fo-Fo isomorphous difference map was performed by the CCP4 package using CAD, SCALEIT and FFT as well as Phaser to calculate phases. Simulated annealing omit map was performed by CNS (Brunger, A. T. (2007), Version 1.2 of the Crystallography and NMR system. Nature Protocols 2, 2728-2733; Diederichs, K. & Karplus, P. A. (1997), Improved R-factors for diffraction data analysis in macromolecular crystallography. Nature structural biology 4, 269-75).

### Crystallographic Analysis of the Phosphorylated LpCopA Mimicry State

The standard procedure of crystallographic data processing in XDS gave a resolution of 2.9 Å (Table 3). An improvement of the diffraction data was made due to by reducing the misfits according to the space group and gave a final resolution that is acceptable to 2.7 Å resolution (Table 2). Initial phases for the beryllium fluoride LpCopA were found using Phaser32 and the (LpCopA-AlF₄⁻) structure as a search model14. The final refinement statistics however showed at 2.7 Å has an outer resolution shell Rfree of over 50%. Therefore, the model was refined to 2.75 Å that gave a more acceptable Rfree value in the outer resolution shell (Table 3).

A comparison of the final structure of the E₂P (BeF₃⁻) state with the previously observed E₂-Pᵢ (AlF₄⁻) model of LpCopA shows a clear movement of the cytoplasmic domains as expected from the analogous structures of SERCA1a. There is a slight movement of TM A and B if the two proteins are superimposed on P-domain (FIG. 16A). In addition, a closing of TM 2 is seen in the E₂P on the cytoplasmic site that closes the extrusion pathway for incoming substrates on the cytoplasmic site. However, the TMD of the E₂P and the E₂-Pᵢ conformational states is rather identical and does not follow the mechanistic features of SERCA1a. This is emphasized on the low root mean square distance (r.m.s.d.) values for the TMD compared to SERCA1a using a superimposition on the overall molecule or the TMD only (Table 4). Thus, LpCopA is not drastically open on the extracellular side as observed for the phosphorylated SERCAa1 state mimicry if one superimposes the two ATPases on their structurally conserved P-domain (FIG. 20).

In addition, the Fo-Fc map as well as corresponding simulated annealing omit maps (SA omit maps) clearly indicate the presence of octahedral coordinated Mg₂⁺ as well as the beryllium fluoride phosphate analog at D426 at the phosphorylation site of the P-domain (FIG. 16B).

Unexpectedly, the map revealed that the β-NAD is bound in the N-domain indicating the reason why the compound has a significant influence on the resolution and quality of the crystals (FIG. 16C). The β-NAD is bound as seen for the AMPPCP and ADP in the N-P-domain structures of CopA from Archaeoglobus fulgidus (AfCopA) (Tsuda, T. & Toyoshima, C. (2009), Nucleotide recognition by CopA, a Cu+-transporting P-type ATPase, The EMBO Journal 28, 1782-1791). The adenosine ring is hydrogen bond coordinated by E459 and S426 and the α-phosphate is coordinated by the invariant histidine (H464) that is associated with the most frequent Wilson disease mutation in Caucasian patients. The β-phosphate of β-NAD seems not to be coordinated by this histidine residue as seen for AMPPCP. Hydrogen bond interactions from waters to the ribose ring have been revealed from the electron density map and are not seen not seen for the NP-domain structure of AfCopA. A simulated annealing omit map confirms the position of this waters (FIG. 16C). The crystal contacts of the BeF₃⁻ state do not necessarily imply that the transmembrane domain is closed due to crystallographic contacts (FIG. 17A). However, the packing in the transmembrane region is much tighter than for the corresponding beryllium fluoride structure of SERCA1a (FIG. 17B) and follows a typical type I crystal packing where the TMDs are packed in a membrane layer46.

The comparison of side chain movements between LpCopA-AlF₄⁻ (E₂-P) and LpCopA-BeF₃⁻ (E₂P) in the transmembrane domain is inappropriate at this resolution and can therefore not be considered. However, a resolution better than 3 Å can reveal some well-ordered water molecules. In the BeF₃⁻ structure most of them have been modeled in the cytoplasmic domains, but there was indication of some waters in the transmembrane domain (FIG. 18) and these have been confirmed by a simulated annealing omit map that was calculated using CNS. The molecular replacement solution showed no clear indication for the heavy metal binding domain and strategy of soaking heavy metals was used to understand the interaction of the ATPase with the HMBD (Table 5). However, no anomalous signal has been observed in the soaked crystals and a mapping of the HMBD was therefore impossible with the present data for the analyzed LpCopABeF₃⁻ structural data.

### Example 3

### LpCopA complexed with MgF₄²⁻

Crystallization of LpCopA Complexed with MgF₄²

Prior to the crystallization experiments the sample was ultracentrifuged for 10 minutes at 100.000×g, the supematant treated with 10 mM NaF, 2 mM MgCl₂, 2 mM EGTA, 10 μM ammonium-tetrathiomolybdate (TTM) as well as the secondary detergents Cymal-6 or deoxy Big CHAP (both between 3-5×CMC, except for seleno-methionine derivatized protein that required lower concentrations).

Crystals were grown at 19° C. using the hanging drop vapor diffusion method with a reservoir solution containing 5% glycerol, 200 mM KCl, 11% PEG 2K MME, 5 mM BME. 1 μL protein and 1 μL precipitant were mixed. Best crystal (with a size of approximately 100 μm×100 μm×20 μm) from these drops were found after 14 days with an addition of 50 mM sodium malonate pH 7.0 to the protein solution. Optimal crystals could be obtained following multiple rounds of optimization using same batch aliquots.

New Insight into the Dephosphorvlation State of LpCopA

In Studies of SERCA1a, the structure of the E₂MgF₄²⁻ state mimics a conformation immediately after the E₂-AlF₄⁻ state in the Post-Albers cycle (Laursen, M., Bublitz, M., Moncoq, K., Olesen, C., Moller, J. V., Young, H. S., Nissen, P. & Morth, J. P. (2009), Cyclopiazonic acid is complexed to a divalent metal ion when bound to the sarcoplasmic reticulum Ca²⁺-ATPase. J Biol Chem 284, 13513-8). For LpCopA, the goal was to understand if the heavy metal binding domain interacts with the protein in this later state. Crystals for this structure have been observed using vapor-diffusion hanging drop crystallization. The crystals appeared within two weeks and had an approximate size of 100 μm×100 μm×20 μm. The obtained crystals diffracted to 3.8 Å (Table 6) and could be improved to 3.6 Å with data processing optimization as described above (Table 3 and Table 7). Initial phases have been obtained by molecular replacement and the final model shows absolutely no differences between the two dephosphorylation structures (LpCopA-MgF₄²⁻ and LpCopA-AlF₄⁻). This is seen from the rather identical r.m.s.d. values for the backbone of the two proteins (Table 4) as well as from a Fo-Fo isomorphous difference map calculated as described by Rould and Carter (Rould, M. A. & Carter, C. W. (2003), Isomorphous difference methods. Macromolecular Crystallography, Pt D 374, 145-163) reprocessing the original data of the AlF₄⁻ into the space group P2₁2₁2₁ (FIG. 19). The R-factor of the two merged data sets calculated by Scalelt from the ccp4 program suit is 0.299 and might be on the high site to analysis it.

### Example 4

### Method for Measuring the ATPase Activity

The activity of the ATPases according to the invention may be measured using the following method, which is based on the Baginsky Method with Bismuth detection (“Bismuth citrate in the quantification of inorganic phosphate and its utility in the determination of membrane-bound phosphates”, Cariani, L. Thomas, J. Brito, and J. R. del Castillo, Analytical Biochemistry 324 (2004), p. 79-83).

| | |
|---|---|
| 1. | Measurements are preferably performed in triplicates. |
| 2. | Pipett 49 μl of Reaction buffer into each well. For testing the inhibitory activity of an |
| | inhibitor a suitable amount of inhibitor may be added. |
| 3. | Start reaction by addition of 1 μl of your sample containing the ATPase and mix |
| | by pipetting |
| 4. | Incubate for 5 min at room temperature (RT) (alternatively incubation can be |
| | performed at higher temperatures, for example 37 degrees Celsius, depending |
| | on the ATPase). Incubation time might need to be adjusted, depending on the |
| | activity of your pump. Further, the ATPase activity may be tested by incubating |
| | samples at different times. |
| 5. | Stop reaction and start color development by addition of 75 μl of Solution II and |
| | mix by pipetting |
| 6. | Incubate for 8 min at RT (alternatively incubation can be performed on ice) |
| 7. | Stabilize the colored complex by adding 125 μl of Solution III |
| 8. | Incubate for 30 min at RT (alternatively incubation can be performed at higher |
| | temperatures, for example 37 degrees Celsius, depending on the ATPase). |
| 9. | Measure the absorption at 710 nm in the plate reader. |
| 10. | Prepare a standard curve using K₂HPO₄ in the nmol range and perform steps 6-10 |

Solution I: (stable)

| | | |
|---|---|---|
| 10% | (w/v) | ammonium molybdate |

Solution II: (prepare fresh every time)

| | | |
|---|---|---|
| 0.43 | g | ascorbic acid → dissolve in 10 ml H₂O |
| 1.48 | ml | 37% HCl (corresponds to 1M HCl) → cool on ice |
| 715 | μl | Solution I |
| 2.145 | ml | 20% SDS → fill up to 15 ml |

Solution III: (stable for some weeks if protected from light)

| | | |
|---|---|---|
| 1.75 | g | bismuth citrate → dissolve in 40 ml H₂O (turbid solution) |
| 4.93 | ml | 37% HCl (corresponds to 1M HCl; solution becomes clear) |
| 1.75 | g | sodium citrate |

2× ATPase activity buffer (this is for Na, K-ATPase):

| | | |
|---|---|---|
| 260 | mM | NaCl |
| 40 | mM | KCl |
| 8 | mM | MgCl₂ |
| 60 | mM | Histidine, pH 7.2-7.4 |

Reaction buffer:
3 mM ATP in 1× ATPase activity buffer
Additionally:
1 mM K₂HPO₄ for the standard curve

The reaction can also be started by addition of ATP instead of starting by the addition of your sample (this is probably better if you want to run a lot of samples in parallel)
You can also leave out the SDS from Solution II and stop the reaction by adding 10 μl 20% SDS and then add 65 μl for color development (this is probably better if you want to run a lot of samples in parallel)

For some types of ATPases, high concentrations of ADP are inhibiting. Therefore, it may be necessary to adjust the incubation time in such a way that a maximum of 10-15% ADP is produced during the incubation time.

To test the inhibitory activity of an inhibitor identified according to the methods as described herein, a suitable amount of inhibitor is added in step 2. The inhibitory activity of the inhibitor is tested by adding different concentrations of inhibitor to each test tube such as at least 0.01 nM, 0.1 nM, 1 nM, 5 nM, 0.01 mM, 0.05 mM, 0.1 mM, 0.5 mM, 1 mM, 2 mM, 5 mM, 10 mM, 25 mM, 50 mM or 100 mM or even more.

Amino acid sequence of Legionella pneumophila
LPG1024
>tr|Q5ZWR1|Q5ZWR1_LEGPH Copper efflux ATPase OS =
Legionella pneumophila subsp. pneumophila
(strain Philadelphia 1/ATCC 33152/DSM 7513).
SEQ ID NO: 1
MKHDHHQGHTHSGKGHACHHEHNSPKTQQASSKMEGPIVYTCPMHPEIRQ
SAPGHCPLCGMALEPETVIVSEVVSPEYLDMRRRFWIALMLTIPVVILEM
GGHGLKHFISGNGSSWIQLLLATPVVLWGGWPFFKRGWQSLKTGQLNMFT
LIAMGIGVAWIYSMVAVLWPGVFPHAFRSQEGVVAVYFEAAAVITTLVLL
GQVLELKAREQTGSAIRALLKLVPESAHRIKEDGSEEEVSLDNVAVGDLL
RVRPGEKIPVDGEVQEGRSFVDESMVTGEPIPVAKEASAKVIGATINQTG
SFVMKALHVGSDTMLARIVQMVSDAQRSRAPIQRLADTVSGWFVPAVILV
AVLSFIVWALLGPQPALSYGLIAAVSVLIIACPCALGLATPMSIMVGVGK
GAQSGVLIKNAEALERMEKVNTLVVDKTGTLTEGHPKLTRIVTDDFVEDN
ALALAAALEHQSEHPLANAIVHAAKEKGLSLGSVEAFEAPTGKGVVGQVD
GHHVAIGNARLMQEHGGDNAPLFEKADELRGKGASVMFMAVDGKTVALLV
VEDPIKSSTPETILELQQSGIEIVMLTGDSKRTAEAVAGTLGIKKVVAEI
MPEDKSRIVSELKDKGLIVAMAGDGVNDAPALAKADIGIAMGTGTDVAIE
SAGVTLLHGDLRGIAKARRLSESTMSNIRQNLFFAFIYNVLGVPLAAGVL
YPLTGLLLSPMIAAAAMALSSVSVIINALRLKRVTL

Lengthy table referenced here
US20150045284A1-20150212-T00001
Please refer to the end of the specification for access instructions.

Lengthy table referenced here
US20150045284A1-20150212-T00002
Please refer to the end of the specification for access instructions.

Lengthy table referenced here
US20150045284A1-20150212-T00003
Please refer to the end of the specification for access instructions.

Lengthy table referenced here
US20150045284A1-20150212-T00004
Please refer to the end of the specification for access instructions.

Lengthy table referenced here
US20150045284A1-20150212-T00005
Please refer to the end of the specification for access instructions.

Lengthy table referenced here
US20150045284A1-20150212-T00006
Please refer to the end of the specification for access instructions.

Lengthy table referenced here
US20150045284A1-20150212-T00007
Please refer to the end of the specification for access instructions.

Lengthy table referenced here
US20150045284A1-20150212-T00008
Please refer to the end of the specification for access instructions.

Lengthy table referenced here
US20150045284A1-20150212-T00009
Please refer to the end of the specification for access instructions.

Lengthy table referenced here
US20150045284A1-20150212-T00010
Please refer to the end of the specification for access instructions.

Lengthy table referenced here
US20150045284A1-20150212-T00011
Please refer to the end of the specification for access instructions.

Lengthy table referenced here
US20150045284A1-20150212-T00012
Please refer to the end of the specification for access instructions.

Lengthy table referenced here
US20150045284A1-20150212-T00013
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20150045284A1). An electronic copy of the table will also be available from the USPTO upon request and payment of the fee set forth in 37 CFR 1.19(b)(3).

2. A crystal comprising a type IB P-type ATPase, wherein said crystal is characterised in having the space group P1.

3. The crystal according to claim 1, wherein said crystal is characterised in having the unit cell parameters a=44 Å±3 Å, b=80 Å±4 Å, c=330 Å±20 Å.

4. The crystal according to any of claims 1 and 2, where the type IB P-type ATPase forms a complex with AlF₄⁻.

5. The crystal according to claim 1, wherein said crystal is characterised in having the unit cell parameters a=44 Å±3 Å, b=73 Å±4 Å, c=329 Å±20 Å.

6. The crystal according to claim 4 wherein said crystal forms a complex with MgF₄²⁻.

7. The crystal according to any of the preceding claims, wherein said crystal is characterised in having the space group P2₁2₁2₁.

8. The crystal according to claim 1, wherein said crystal is characterised in having the unit cell parameters a=242 Å±15 Å, b=71 Å±4 Å, c=72 Å±4 Å.

9. The crystal according to claim 7, where the type IB P-type ATPase forms a complex with BeF₃⁻.

10. The crystal according to any of claims 1, 7 and 8, wherein said crystal is characterised in having the space group C₂.

11. The crystal according to any of the preceding claims, wherein said ATPase is a bacterial type IB P-type ATPase.

12. The crystal according any of the preceding claims, wherein said type IB P-type ATPase is a Legionella pneumophila type IB P-type ATPase.

13. The crystal according claim 5, wherein said type IB P-type ATPase is the LPG1024 (SEQ ID NO:1) Legionella pneumophila ATPase or an ATPase having at least 85% sequence identity with SEQ ID NO:1 or a functional homologue thereof.

14. The crystal according any of the preceding claims, wherein said type IB P-type ATPase is a Cu⁺ ATPase.

15. The crystal according to any of the preceding claims, where the type IB P-type ATPase forms a complex with an organic compound selected from the group consisting of: ATP, ATP analogues, ADP and ADP analogues.

16. The crystal according to any of the preceding claims, which effectively diffracts x-rays for the determination of the atomic structure of the protein to a resolution better than 3,5 Å.

17. A method for purification of a type IB P-type ATPase comprising the following steps:
d. obtaining a composition comprising a type IB P-type ATPase,
e. solubilising said type IB P-type ATPase using a polyoxyethylene lauryl ether and/or a maltoside-based detergent,
f. purifying said type IB P-type ATPase.

18. A method of growing a crystal comprising a type IB P-type ATPase according to claim 1, comprising the steps of:
d. obtaining a composition comprising a type IB P-type ATPase,
e. growing type IB P-type ATPase crystals in a crystallization environment including PEG and
f. obtaining crystals comprising a type IB P-type ATPase.

19. The method according to claim 12, further comprising a step of treating said composition comprising a type IB P-type ATPase with at least 0.1 mg/ml of one or more lipids before growing the type IB P-type ATPase crystals.

20. The method according to 13, wherein said type IB P-type ATPase is treated with dioleoyl-phosphatidylcholine before growing the crystals.

21. The method according to claim 12, wherein step b further comprises growing type IB P-type ATPase crystals by vapour diffusion from hanging drops with a reservoir buffer containing PEG.

22. The methods according to claim 20, wherein the concentration of PEG is 2-12% (w/v), such as 4-9% (w/v) or 5-7% (w/v)

23. The method according to claim 12, wherein step b further comprises growing type IB P-type ATPase crystals by vapour diffusion from hanging drops with a reservoir buffer containing PEG 2000 MME

24. The methods according to claim 22, wherein the concentration of PEG 2000 MME is 2-20% (w/v), such as 6-16% (w/v) or 10-15% (w/v).

25. The method according to claim 20, wherein the reservoir buffer comprises 5-7% (w/v) PEG, 80-200 mM NaCl, 3% v/v t-BuOH and 5 mM BME.

26. The method according to claim 20, wherein the reservoir buffer comprises 14% (w/v) PEG 2000 MME, 200 mM KCl, 3% (v/v) t-BuOH and 5 mM BME.

27. The method according to claim 20, wherein the reservoir buffer comprises 11% (w/v) PEG 2000 MME, 200 mM KCl and 5 mM BME.

28. The methods according to any of claims 12-17, wherein PEG is PEG 6000.

29. Use of a crystal according to any one of claims 1-10 for determination of the three dimensional structure of said type IB P-type ATPase.

30. Use of a crystal according to any one of claims 1-10 for identifying inhibitors of a type IB P-type ATPase.

31. The use according to claim 19, further comprising a step of contacting said crystal with one or more compounds.

32. Use of atomic coordinates as presented in Table 1 or atomic coordinates selected from a three-dimensional structure that deviates from the three-dimensional structure as presented in Table 1 by a root mean square deviation over protein backbone atoms of not more than 3 Å, in a method for identifying a inhibitor of a type IB P-type ATPase.

33. A method for identifying an inhibitor of a type IB P-type ATPase by determining binding interactions between the inhibitor and a set of binding interaction sites in said type IB P-type ATPase comprising the steps of:
d. generating the spatial structure of the type IB P-type ATPase on a computer screen using atomic coordinates as presented in Table 1 or atomic coordinates selected from a three-dimensional structure that deviates from the three-dimensional structure as presented in Table 1 by a root mean square deviation over protein backbone atoms of not more than 3 Å,
e. generating the spatial structure of inhibitors on the computer screen, and
f. selecting inhibitors that can bind to at least one amino acid residue of the set of binding interaction sites with out steric interference.

34. A computer-assisted method for identifying inhibitors of a type IB P-type ATPase using a programmed computer processor, a data storage system, a data input devise and a data output devise comprising the following steps:
a. putting into the programmed computer through said input device data comprising: a subset of the atoms of a type IB P-type ATPase, thereby generating a criteria data set, wherein the atomic coordinates are selected from the three-dimensional structure as presented in Table 1 or atomic coordinates selected from a three-dimensional structure that deviates from the three-dimensional structures as presented in Table 1 by a root mean square deviation over protein backbone atoms of not more than 3 Å,
b. comparing, using said processor, the criteria data set to a computer data base of low molecular weight organic chemical structures stored in the data storage system; and
e. selecting from said data base, using computer methods, a chemical structure having a portion that is structurally complementary to the criteria data set and being free of steric interference with the type IB P-type ATPase and/or
d. constructing using computer methods a model for a chemical structure having a portion that is structurally complementary to the criteria data set and being free of steric interference with the type IB P-type ATPase.

35. The method according to claims 23-24, wherein the criteria data set or the binding interaction sites may comprise one or more amino acid residues selected from the group comprising: Glu189, Met711, Met100 and Glu99 of SEQ ID NO:1.

36. The method according to claims 23-24, wherein the criteria data set or the binding interaction sites may comprise one or more amino acid residues selected from the group comprising: Cys382, Pro383, Cys384, Tyr688, Asn689, Met717, Ser721 of SEQ ID NO:1.

37. The method according to claims 23-24, wherein the criteria data set or the binding interaction sites may comprise one or more amino acid residues selected from the group comprising: Thr277, Gly278, Glu279, Asp426, Lys427, Thr428, Gly429, Thr430 and Thr432 of SEQ ID NO:1.

38. The method according to claims 23-24, wherein the criteria data set or the binding interaction sites set may comprise one or more amino acid residues selected from the group comprising: Met148, Asp337 and Glu205 of SEQ ID NO:1.

39. The method according to claims 23-24, wherein the criteria data set may comprise one or more amino acid residues selected from the group comprising: Gly129 and Gly130 of SEQ ID NO:1.

40. A method for identifying an inhibitor capable of inhibiting a type IB P-type ATPase, said method comprising the following steps:
a. identifying an inhibitor using atomic coordinates in conjunction with computer modelling, wherein said atomic coordinates are the atomic coordinates presented in Table 1 or wherein the atomic coordinates are selected from a three-dimensional structure that deviates from the three-dimensional structures presented in Table 0.1 by a root mean square deviation over protein backbone atoms of not more than 3 Å, by docking inhibitors into a set of binding interaction sites in a type IB P-type ATPase generated by computer modelling and selecting a inhibitor capable of binding to at least one amino acid in said type IB P-type ATPase,
b. providing said inhibitor and said type IB P-type ATPase,
c. contacting said inhibitor with said type IB P-type ATPase and
d. detecting inhibition the activity of said type IB P-type ATPase by the inhibitor.

41. The method according to claim 30, wherein docking of inhibitor molecules is performed by employing the type IB P-type ATPase crystal defined by atomic coordinates presented in Table 1 and such that said inhibitor is capable of binding to at least three amino acid in the type IB P-type ATPase.

42. A method for identifying an inhibitor capable of inhibiting a type IB P-type ATPase, said method comprising the following steps:
a. introducing into a computer information derived from atomic coordinates presented in Table 1 or atomic coordinates selected from a three-dimensional structure that deviates from the three-dimensional structures as presented in Table 1 by a root mean square deviation over protein backbone atoms of not more than 3 Å,
b. generating a three-dimensional structure using said atomic coordinates,
c. superimposing a model of an inhibitor on said three-dimenssional structure;
d. assessing the possibility of binding and the absence of steric interference of the inhibitor with the type IB P-type ATPase;
e. incorporation said inhibitor in an activity assay of said type IB P-type ATPase and
f. determining whether said inhibitor inhibits the activity of said type IB P-type ATPase.

43. The method according to any of the claims 30-32, wherein information is derived from the atomic coordinates of at least one of the amino acid residues selected from the group comprising: Glu189, Met711, Met100 and Glu99 of SEQ ID NO:1.

44. The method according to any of the claims 30-32, wherein information is derived from the atomic coordinates of at least one of the amino acid residues selected from the group comprising: Met148, Asp337 and Glu205 of SEQ ID NO:1.

45. The method according to any of the claims 30-32, wherein information is derived from the atomic coordinates of at least one of the amino acid residues selected from the group comprising: Cys382, Pro383, Cys384, Tyr688, Asn689, Met717 and Ser721 of SEQ ID NO:1.

46. The method according to any of the claims 30-32, wherein information is derived from the atomic coordinates of at least one of the amino acid residues selected from the group comprising: Thr277, Gly278, Glu279, Asp426, Lys427, Thr428, Gly429, Thr430 and Thr432 of SEQ ID NO:1.

47. The method according to any of the above claims 23-36, wherein the atomic coordinates are determined to a resolution of at least 3,5 Å.

48. The method according to any of the claims 23-37, wherein a library of small organic molecules are screened.

49. The method according to any of the claims 23-37, wherein a library of peptide inhibitors are screened.

50. A method for identifying a selective peptide inhibitor of a type IB P-type ATPase comprising the following steps:
f. identification of a inhibitor of a type IB P-type ATPase according to any of the claims,
g. contacting the peptide inhibitor with said type IB P-type ATPase,
h. contacting the peptide inhibitor with a different type IB P-type ATPase,
i. detecting inhibition of type IB P-type ATPase activity of said type IB P-type ATPase by the inhibitor and
j. detecting activity of said different type IB P-type ATPase in the presence of said inhibitor.

51. An inhibitor of a type IB P-type ATPase, wherein said inhibitor is identified according to the methods of claims 23-39.

52. The inhibitor according to claim 41, wherein said inhibitor is capable of inhibiting growth of bacteria having type IB P-type ATPases in their cell membrane.

53. The inhibitor according to claim 42, wherein said bacteria are pathogenic bacteria.

54. Use of the inhibitor according to claims 41-43 for treatment of an individual infected with pathogenic bacteria having type IB P-type ATPases in their cell membrane.

55. A method for treatment of an individual infected with pathogenic bacteria having type IB P-type ATPases in their cell membrane, said method comprising administering to said individual an inhibitor of a type IB P-type ATPase.


SEQUENCE LISTING (1 sequence)

SEQ ID NO: 1 - protein, 736 aa, Legionella pneumophila
MKHDHHQGHTHSGKGHACHHEHNSPKTQQASSKMEGPIVYTCPMHPEIRQSAPGHCPLCGMALEPETVTVSEVVSPEYLDMRRRFWIALMLTIPVVILEMGGHGLKHFISGNGSSWIQLLLATPVVLWGGWPFFKRGWQSLKTGQLNMFTLIAMGIGVAWIYSMVAVLWPGVFPHAFRSQEGVVAVYFEAAAVITTLVLLGQVLELKAREQTGSAIRALLKLVPESAHRIKEDGSEEEVSLDNVAVGDLLRVRPGEKIPVDGEVQEGRSFVDESMVTGEPIPVAKEASAKVIGATINQTGSFVMKALHVGSDTMLARIVQMVSDAQRSRAPIQRLADTVSGWFVPAVILVAVLSFIVWALLGPQPALSYGLIAAVSVLIIACPCALGLATPMSIMVGVGKGAQSGVLIKNAEALERMEKVNTLVVDKTGTLTEGHPKLTRIVTDDFVEDNALALAAALEHQSEHPLANAIVHAAKEKGLSLGSVEAFEAPTGKGVVGQVDGHHVAIGNARLMQEHGGDNAPLFEKADELRGKGASVMFMAVDGKTVALLVVEDPIKSSTPETILELQQSGIEIVMLTGDSKRTAEAVAGTLGIKKVVAEIMPEDKSRIVSELKDKGLIVAMAGDGVNDAPALAKADIGIAMGTGTDVAIESAGVTLLHGDLRGIAKARRLSESTMSNIRQNLFFAFIYNVLGVPLAAGVLYPLTGLLLSPMIAAAAMALSSVSVIINALRLKRVTL